SEQUENCE LISTING (41 sequences)

SEQ ID NO: 1 - protein, 805 aa, Homo sapiens [sequence omitted]

SEQ ID NO: 2 - protein, 598 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 723 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 195 aa, synthetic construct
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 5 - protein, 196 aa, synthetic construct
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 6 - protein, 825 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 950 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 855 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1578 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 10 - protein, 1578 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 11 - protein, 598 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 12 - protein, 723 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 13 - protein, 232 aa, synthetic construct
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 17 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 18 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 21 - protein, 279 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 22 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 26 - protein, 235 aa, synthetic construct; identical to SEQ ID NO: 18

SEQ ID NO: 27 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 32 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 34 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 36 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 37 - protein, 231 aa, synthetic construct
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 38 - protein, 107 aa, synthetic construct [sequence omitted]

SEQ ID NO: 39 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

(19) United States
(12) Patent Application Publication
(10) Pub. No.: US 2024/0252601 A1
(43) Pub. Date: Aug. 1, 2024

(54) ACE2 FUSION PROTEINS AND USES THEREOF

(71) Applicant: Regeneron Pharmaceuticals, Inc., Tarrytown, NY (US)

(72) Inventors: Nianshuang WANG, Cos Cob, CT (US); Yang SHEN, Scarsdale, NY (US); Alina BAUM, Pleasantville, NY (US); Christos KYRATSOUS, Irvington, NY (US); Chia-Yang LIN, Scarsdale, NY (US)

(73) Assignee: Regeneron Pharmaceuticals, Inc., Tarrytown, NY (US)

(21) Appl. No.: 18/395,175

(22) Filed: Dec. 22, 2023

Related U.S. Application Data
(60) Provisional application No. 63/477,058, filed on Dec. 23, 2022.
(60) Provisional application No. 63/507,593, filed on Jun. 12, 2023.

(51) Int. Cl. A61K 38/48; A61P 31/14; C12N 9/48

(52) U.S. Cl. CPC A61K 38/48; A61P 31/14; C12N 9/485; C12Y 304/17023; C07K 2319/30

(57) ABSTRACT

The present disclosure provides ACE2 fusion proteins, comprising multimerization moieties linked to ACE2 moieties, that specifically bind to RBD regions of SARS-CoV and/or SARS-CoV-2. The present disclosure further relates to the methods of producing the ACE2 fusion proteins, pharmaceutical compositions comprising of the ACE2 fusion proteins, and methods of use of the ACE2 fusion proteins to treat conditions associated with SARS-CoV and SARS-CoV-2 infections, such as COVID-19.

### 1. CROSS-REFERENCE TO RELATED APPLICATIONS

This application claims the priority benefit of U.S. provisional application No. 63/477,058, filed Dec. 23, 2022, and U.S. provisional application No. 63/507,593, filed Jun. 12, 2023, the contents of each of which are incorporated herein in their entireties by reference thereto.

### 2. SEQUENCE LISTING

The instant application contains a Sequence Listing which has been submitted electronically and is hereby incorporated by reference in its entirety. Said copy, created on Dec. 20, 2023, is named RGN-026US_SL.xml and is 52,446 bytes in size.

### 3. BACKGROUND

Severe acute respiratory syndrome coronavirus 2 (SARS-CoV-2) is an enveloped, positive-sense, single-stranded RNA virus of the genus Betacoronavirus, which also includes SARS-CoV, Middle East respiratory syndrome coronavirus (MERS-COV), human coronavirus (HCoV)-OC43, and HCoV-HKU1 (Jackson et al., 2022, Nat Rev Mol Cell Biol. 23(1): 3-20). SARS-CoV-2 causes COVID-19, a potentially life-threatening disease which was first characterized in late 2019 and escalated into a global pandemic in early 2020.

SARS-CoV-2 shares ˜80% sequence identity with SARS-CoV and both viruses rely on their interaction with the angiotensin-converting enzyme 2 (ACE2) for cellular entry. ACE2 is an enzyme expressed on the extracellular surface of many types of cells such as respiratory epithelia, cardiomyocytes, endothelial cells, artery smooth muscle cells, among others (Beyerstedt et al., 2021, Eur J Microbiol Infect Dis. 40(5): 905-919). It is primarily involved in vascular tone regulation by catalyzing the cleavage of the angiotensin precursors Ang I and/or Ang II, which is essential for the maturation of angiotensin (Yan et al., 2020, Science. 367:1444-1448). SARS-CoV and SARS-CoV-2 compete with these precursors for ACE2 binding and use this interaction to enter host cells. Nevertheless, SARS-Cov-2 has been shown to have a higher affinity to human ACE2 (hACE2) and bind more strongly to soluble hACE2 than SARS-CoV (Beyerstedt et al., 2021, Eur J Microbiol Infect Dis. 40(5): 905-919). This enhanced affinity of SARS-CoV-2 to hACE2 may underlie its high infectivity.

Currently, several vaccines against SARS-CoV-2 are used to prevent manifestation of severe disease. However, vaccination rates vary among populations, and even in areas with high rates of vaccination, breakthrough infections leading to COVID-19 have been observed in individuals who have been immunized against SARS-CoV-2. Previous SARS-CoV-2 infections don't seem to provide a complete immunity against future infections either, as some individuals have been diagnosed with COVID-19 multiple times. Moreover, SARS-CoV-2 infection in some individuals leads to a prolonged disease associated with persistence of one or more symptoms of COVID-19 for weeks to months after the clearance of infection. These observations underscore the serious population health threat posed by COVID-19 and the need to fight SARS-CoV-2 infections with effective treatments.

Biologic treatments such as monoclonal antibodies may become obsolete with the rapid emergence of new SARS-CoV-2 variants. Small molecule treatments such as Paxlovid—a combination of the oral antiviral drugs nirmatrelvir and ritonavir—are associated with a ‘Paxlovid rebound’ effect in which the virus reemerges. Callaway, Nature (News), 11 Aug. 2022. Hence, there remains a need to develop novel treatments that will be effective against different strains of SARS-CoV-2.

### 4. SUMMARY

The present disclosure relates to ACE2 fusion proteins for inhibiting the interaction between coronaviruses and host cells. The ACE2 fusion proteins of the disclosure lack the drawbacks of vaccines and therapies specific to a particular coronavirus strain.

The ACE2 fusion proteins of the disclosure generally comprise one or more polypeptide chains having the formula [A1]-[L1]-[MM1]-[L2]-[MM2]-[L3]-[A2]-[L4]-[MM3], wherein [A1] represents an ACE2 moiety; [L1] represents a linker; [MM1] represents a multimerization moiety; [L2] represents a linker; [MM2] represents a multimerization moiety; [L3] represents a linker; [A2] represents an ACE2 moiety; [L4] represents a linker; and [MM3] represents a multimerization moiety, and wherein each of [L1], [L2], [L3] and [L4] may be absent, one or two but not all three of [MM1], [MM2] and [MM3] may be absent, and one but not both of [A1] and [A2] may be absent.

In some embodiments, the ACE2 fusion proteins of the disclosure comprise one or more polypeptide chains having the formula [A1]-[L1]-[MM1]-[L2]-[A2], wherein [A1] represents a first ACE2 moiety; [L1] represents a first linker; [MM1] represents a first multimerization moiety; [L2] represents a second linker; and [A2] represents a second ACE2 moiety, wherein [L1] and [L2] are optional and one of [A1] and [A2] is optional.

In some embodiments, the ACE2 fusion proteins of the disclosure comprise one or more polypeptide chains having the formula [A3]-[L3]-[MM2]-[A1]-[L1]-[MM1]-[L2]-[A2], wherein [A1] represents a first ACE2 moiety; [L1] represents a first linker; [MM1] represents a first multimerization moiety; [L2] represents a second linker; [A2] represents a second ACE2 moiety, [A3] represents a third ACE2 moiety; [L3] represents a third linker; and [MM2] represents a second multimerization moiety, wherein [L1], [L2] and [L3] are optional and one of [A1] and [A2] is optional. Optionally, each polypeptide chain having the formula [A3]-[L3]-[MM2]-[A1]-[L1]-[MM1]-[L2]-[A2] is associated with a polypeptide chain having the formula [A4]-[L4]-[MM3]-[L5]-[A5], wherein; [A4] represents a fourth ACE2 moiety; [L4] represents a fourth linker; [MM3] represents a third multimerization moiety that is capable of associating with [MM2]; [L5] is a fifth linker; [A5] is a fifth ACE2 moiety; and wherein [L4], [L5] and [A5] are optional.

Exemplary configurations and structures of ACE2 fusion proteins of the disclosure are depicted in FIGS. 2A-2J and described in Section 6.2 and numbered embodiments 1 to 224.

ACE2 moieties suitable for incorporation into the ACE2 fusion proteins of the disclosure are described in Section 6.3 and defined in numbered embodiments 2 to 23 (e.g., for [A1]) and 76 to 97 (e.g., for [A2]).

Linker moieties suitable for incorporation into the ACE2 fusion proteins of the disclosure are described in Section 6.5 and defined in numbered embodiments 26 and 27 (e.g., for [L1]); 51 and 52 (e.g., for [L2]); 74 and 74 (e.g., for [L3]); and 100 and 101 (e.g., for L4).

Multimerization moieties suitable for incorporation into the ACE2 fusion proteins of the disclosure are described in Section 6.4 and defined in numbered embodiments 30 to 48 (e.g., for [MM1]); 55 to 71 (e.g., for [MM2]); and 104 to 107 (e.g., for [MM3]).

The present disclosure further provides nucleic acids encoding the ACE2 fusion proteins of the disclosure, host cells engineered to express the ACE2 fusion proteins of the disclosure, and recombinant methods for the production of the ACE2 fusion proteins of the disclosure. Such nucleic acids, host cells and production methods are described in Section 6.6 and numbered embodiments 132 to 134.

The present disclosure further provides pharmaceutical compositions comprising the ACE2 fusion proteins of the disclosure as well as methods of their use in therapy. Pharmaceutical compositions are described in Section 6.7 and numbered embodiment 135. Methods of use of the ACE2 fusion proteins and pharmaceutical compositions are described in Section 6.8 and numbered embodiments 136 to 144.

Other features and advantages of aspects of the fusion proteins of the present disclosure will become apparent from the following more detailed description, taken in conjunction with the accompanying drawings.

### 5. BRIEF DESCRIPTION OF THE FIGURES

FIGS. 1A-1B show the structure and domains of the ACE2 protein. FIG. 1A is a cartoon representation of the extracellular portion of human ACE2, shown in a complex with the receptor binding domain (RBD) of SARS-CoV-2 spike (S) protein. The soluble, extracellular portion of ACE2 includes an N-terminal peptidase domain (PD) corresponding to amino acids 18 to 615 of human ACE2, and the neck domain (ND) of the collectrin-like domain (CLD), corresponding to amino acids 616-740 of human ACE2. The peptide binding cavity on the outer surface of ACE2-PD is directly involved in RBD binding whereas ACE2-ND is involved in ACE2 dimerization. FIG. 1B shows a diagram of the full-length ACE2 protein with its domains presented in the N- to C-terminal direction.

FIGS. 2A-2J illustrate exemplary ACE2 fusion protein constructs of the disclosure, where:
(1) and (2) represent an ACE2 moiety, for example as described in Section 6.3, where:
(1) represents the PD region of an ACE2 moiety, which can be wildtype or contain one or more mutations, e.g., amino acid substitutions, deletions or insertions. In some embodiments, the substitutions increase affinity to RBD and/or decrease ACE2 catalytic activity, for example as set forth in Section 6.3;
(2) represents the ND region of an ACE2 moiety, which can be wildtype or contain one one or more one or more mutations, e.g., amino acid substitutions, deletions or insertions;
(3) represents an optional linker connecting the ACE2 moiety to the hinge region at the N-terminus of an Fc domain;
(4) represents the hinge region of an immunoglobulin Fc domain, e.g., an IgG hinge domain, which in various embodiments is an IgG1 or an IgG4 hinge domain or a chimeric hinge domain, e.g., as set forth in Section 6.5.1;
(5) represents an Fc domain of an immunoglobulin, e.g., an IgG such as an IgG1 or an IgG4, which typically includes a CH2 domain, CH3 domain, and optionally a hinge domain, e.g., as set forth in Section 6.4;
(6) represents an optional linker connecting the C-terminus of an Fc domain to an ACE2 moiety, which can be wildtype or contain one or more mutations, e.g., amino acid substitutions, deletions or insertions;
(7) represents a region comprising a CH1 domain and a CL domain of an immunoglobulin;
(8) represents an optional linker connecting an ACE2 moiety to the N-terminus of a CH1 or a CL domain; and
(9) represents an optional linker connecting an ACE2 moiety to the C-terminus of a CH1 or a CL domain, on a polypeptide chain lacking an Fc domain (and which pairs to a corresponding CL or CH1 domain on the polypeptide chain comprising an Fc domain).

FIG. 2A shows an exemplary tetravalent ACE2 fusion protein, with ACE2 moieties N and C-terminal to an IgG Fc domain, with the ACE2 moiety N-terminal to the Fc domain comprising a ND and the ACE2 moiety C-terminal to the Fc domain lacking a ND. FIG. 2B shows an exemplary bivalent ACE2 fusion construct with an ACE2 moiety only N-terminal to the Fc domain. FIGS. 2C and 2D show bivalent ACE2 fusion proteins without a linker between the ACE2 domain and the hinge domain in the case of FIG. 2C, and with a linker between the ACE2 domain and the hinge domain in the case of FIG. 2D. FIG. 2E shows an exemplary tetravalent ACE2 fusion protein with ACE2 moieties both N- and C-terminal to the Fc domain and lacking a ND. FIG. 2F shows an exemplary hexavalent construct with ACE2 moieties comprising ND located N-terminal to the regions comprising a CH1 and a CL domain and ACE2 moieties lacking ND located C-terminal to the Fc domain. FIG. 2G shows an exemplary octavalent construct with ACE2 moieties comprising ND located N-terminal to the regions comprising a CH1 and a CL domain, ACE2 moieties lacking ND C-terminal to the CH1-CL regions, and additional ACE2 moieties lacking ND C-terminal to the Fc domain. FIG. 2H shows an exemplary octavalent construct with ND-comprising ACE2 moieties N-terminal to regions comprising a CH1 and a CL domain located N-terminal to the Fc domain and ND-comprising ACE2 moieties N-terminal to the regions comprising a CH1 and a CL domain located C-terminal to the Fc domain. FIG. 2I shows an exemplary tetravalent construct with ND-comprising ACE2 moieties N-terminal to the regions comprising a CH1 and a CL domain, located C-terminal to the Fc domain. FIG. 2J shows an exemplary tetravalent construct with ND-comprising ACE2 moieties N-terminal to the regions comprising a CH1 and a CL domain, located N-terminal to the Fc domain.

FIG. 3 shows representative 4-12% non-reducing SDS-PAGE of ACE2-Fc fusion constructs (“ACE2-Fc fusion proteins” or “ACE2-Fc fusion constructs” refers to ACE2 fusion proteins in which the multimerization moieties are Fc domains) expression in culture medium. The gel shows two tetravalent ACE2-Fc (IgG) fusion constructs (the term “ACE-Fc (IgG)” refers to ACE2 fusion proteins in which the multimerization moieties are IgG-based Fc domains) in media samples collected from transfected Expi 293-F cells. Each well was loaded with 20 μL culture medium.

FIGS. 4A-4F depict the results of the purification of tetravalent ACE2-Fc (IgG) constructs. FIG. 4A shows the size exclusion chromatography (SEC) profile of the three elution fractions, F1, F2, and F3, of the supernatant collected from Expi 293-F cells transfected with ACE2 (740/615)-Fc (IgG). FIG. 4B shows the size exclusion chromatography (SEC) profile of the three elution fractions, F1, F2, and F3, of the supernatant collected from FreeStyle™ 293-F cells transfected with ACE2 v2.4 (740/615)-Fc (IgG). FIGS. 4C and 4D show the results of SDS-PAGE analyses of ACE2 (740/615)-Fc (IgG) and ACE2 v2.4 (740/615)-Fc (IgG), respectively, under reducing and non-reducing conditions. FIGS. 4E and 4F show the analytical SEC profiles of individual fractions, F1, F2, and F3 of ACE2 (740/615)-Fc (IgG) and ACE2 v2.4 (740/615)-Fc (IgG) respectively.

FIGS. 5A-5C show the extent of neutralization of SARS-CoV2 pseudovirus and two variants by different ACE2-Fc (IgG) constructs by a luminescence assay. FIG. 5A illustrates the neutralization potencies of each tested ACE2-Fc (IgG) construct against the SARS-CoV2 pseudovirus D614G, whereas FIGS. 5B and 5C illustrate their neutralization potencies against SARS-CoV2 Omicron BA. 1 and BA.2 variants, respectively.

FIGS. 6A-6F show the extent of neutralization of SARS-CoV2 pseudovirus and five variants by different bivalent and tetravalent ACE2-Fc (IgG) constructs by a luminescence assay. FIG. 6A illustrates the neutralization potencies of each tested ACE2-Fc (IgG) construct against the SARS-CoV2 pseudovirus D614G. FIG. 6B illustrates the neutralization potencies of each tested ACE2-Fc (IgG) construct against SARS-CoV2 Omicron variant BA. 1. FIG. 6C illustrates the neutralization potencies of each tested ACE2-Fc (IgG) construct against SARS-CoV2 Omicron variant BA.2. FIG. 6D illustrates the neutralization potencies of each tested ACE2-Fc (IgG) construct against SARS-CoV2 Omicron variant BA.2.12.1. FIG. 6E illustrates the neutralization potencies of each tested ACE2-Fc (IgG) construct against SARS-CoV2 Omicron variant BA.4/BA.5. FIG. 6F illustrates the neutralization potencies of each tested ACE2-Fc (IgG) construct against SARS-CoV2 Omicron variant BA.4.6.

FIGS. 7A-7F show the effect of ACE2-Fc (IgG) valency on neutralization of SARS-CoV2 pseudovirus and five variants by a luminescence assay, wherein the potencies of bivalent, tetravalent, hexavalent, and octavalent constructs were evaluated. FIG. 7A illustrates the neutralization potencies of each tested ACE2-Fc (IgG) construct against the SARS-CoV2 pseudovirus D614G. FIG. 7B illustrates the neutralization potencies of each tested ACE2-Fc (IgG) construct against SARS-CoV2 Omicron variant BA. 1. FIG. 7C illustrates the neutralization potencies of each tested ACE2-Fc (IgG) construct against SARS-CoV2 Omicron variant BA.2. FIG. 7D illustrates the neutralization potencies of each tested ACE2-Fc (IgG) construct against SARS-CoV2 Omicron variant BA.2.12.1. FIG. 7E illustrates the neutralization potencies of each tested ACE2-Fc (IgG) construct against SARS-CoV2 Omicron variant BA.4/BA.5. FIG. 7F illustrates the neutralization potencies of each tested ACE2-Fc (IgG) construct against SARS-CoV2 Omicron variant BA.4.6.

FIGS. 8A-8F show the extent of neutralization of SARS-CoV2 pseudovirus and five variants by a luminescence assay, wherein the potencies of bivalent (×2), tetravalent (×4), hexavalent (×6), and octavalent (×8) constructs were evaluated. FIG. 8A illustrates the percent neutralization potencies of each tested ACE2-Fc (IgG) construct against the SARS-CoV2 pseudovirus D614G. FIG. 8B illustrates the neutralization potencies of each tested ACE2-Fc (IgG) construct against SARS-CoV2 Omicron variant BA. 1. FIG. 8C illustrates the neutralization potencies of each tested ACE2-Fc (IgG) construct against SARS-CoV2 Omicron variant BA.2. FIG. 8D illustrates the neutralization potencies of each tested ACE2-Fc (IgG) construct against SARS-CoV2 Omicron variant Alpha-468. FIG. 8E illustrates the neutralization potencies of each tested ACE2-Fc (IgG) construct against SARS-CoV2 Omicron variant BA.2.12.1. FIG. 8F illustrates the neutralization potencies of each tested ACE2-Fc (IgG) construct against SARS-CoV2 Omicron variant BA.4/BA.5.

FIGS. 9A-9G show the extent of neutralization of SARS-CoV2 pseudovirus and SARS-CoV2 Omicron variant XBB1.5 by a luminescence assay, wherein the potencies of various bivalent, tetravalent, hexavalent, and octavalent constructs were evaluated. FIG. 9A displays the construct names and structures associated with the symbols used in FIGS. 9B-9G. FIG. 9B shows the percent neutralization potencies of bivalent, tetravalent, and hexavalent constructs against SARS-CoV2 pseudovirus. FIG. 9C shows the percent neutralization potencies of hexavalent and octavalent constructs against SARS-CoV2 pseudovirus. FIG. 9D shows the ACE2 valency adjusted percent neutralization potencies of all constructs displayed in FIGS. 9B and 9C against SARS-CoV2 pseudovirus. FIG. 9E shows the percent neutralization potencies of bivalent, tetravalent, and hexavalent constructs against SARS-CoV2 Omicron variant XBB1.5. FIG. 9F shows the percent neutralization potencies of hexavalent and octavalent constructs against SARS-CoV2 Omicron variant XBB1.5. FIG. 9G shows the ACE2 valency adjusted percent neutralization potencies of all constructs displayed in FIGS. 9E and 9F against SARS-CoV2 Omicron variant XBB1.5. REGEN-COV (REGN10987/REGN10933) and (2x) N2 were included as controls in all assessments.

FIGS. 10A-10C show the differences in neutralization potencies of two SARS-CoV2 variants, wherein the potencies of four ACE2-Fc (IgG) constructs were evaluated. FIG. 10A displays the construct names and structures associated with the symbols used in FIGS. 10B-10C. FIG. 10B shows the percent neutralization potencies of constructs (4x) N2/C2, (6x) N4/C2, and (4x) N4 against SARS-CoV2 Omicron variant BA.2.86. FIG. 10C shows the percent neutralization potencies of constructs (4x) N2/C2, (6x) N4/C2, and (4x) N4 against SARS-CoV2 Eris subvariant EG.5.1. REGEN-COV (REGN10987/REGN10933) and (2x) N2 were included as controls in all assessments.

### 6. DETAILED DESCRIPTION

### 6.1. Definitions

As used herein, the following terms are intended to have the following meanings:

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

ACE2 Moiety: The term “ACE2 moiety” refers to a moiety comprising an amino acid sequence that has at least 70% sequence identity to an extracellular portion of human ACE2 that is capable of binding the RBD of SARS-CoV or SARS-CoV-2 RBD, for example an amino acid sequence having at least 70% sequence identity to the peptidase domain (PD) of human ACE2. In some embodiments, an ACE2 moiety comprises an amino acid sequence having at least 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, or 99% identity to the peptidase domain of human ACE2. In further embodiments, the ACE2 moiety comprises an amino acid sequence having at least 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, or 99% identity to the peptidase and neck domains of human ACE2. Typically, the ACE2 moiety lacks a transmembrane domain. Exemplary ACE moieties are set forth in Section 6.3.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Associated: The term “associated” in the context of an ACE2 fusion protein refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional ACE2 fusion protein.

Examples of associations that might be present in an ACE2 fusion protein of the disclosure include (but are not limited to) associations between Fc domains to form an Fc region (e.g., as described in Section 6.4.1) or a CH1 domain and a CL domain (e.g., as described in Section 6.4.2.

Bivalent: The term “bivalent” as used herein refers to an ACE2 fusion protein comprising two ACE2 moieties, whether in the same polypeptide chain or on different polypeptide chains. The two ACE2 moieties can be the same or different.

COVID-19: The term “COVID-19” is the abbreviation of “Coronavirus disease 2019” and refers to the infectious disease caused by SARS-CoV-2 infection. Patients with COVID-19 may experience a wide range of symptoms ranging from mild to severe, which may include but are not limited to, fever, chills, cough, shortness of breath, difficulty breathing, fatigue, muscle aches, body aches, headache, loss of smell, loss of taste, sore throat, congestion, runny nose, nausea, and diarrhea.

EC50: The term “EC50” refers to the half maximal effective concentration of a molecule (such as an ACE2 fusion protein) which induces a response halfway between the baseline and maximum after a specified exposure time. The EC50 essentially represents the concentration of an ACE2 fusion protein where 50% of its maximal effect is observed. In certain embodiments, the EC50 value equals the concentration of an ACE2 fusion protein that gives half-maximal virus or pseudovirus neutralization in an assay as described in Section 8.1.3.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Hexavalent: The term “hexavalent” as used herein refers to an ACE2 fusion protein comprising six ACE2 moieties, whether in the same polypeptide chain or on different polypeptide chains (e.g., on two or four polypeptide chains). The six ACE2 moieties can be the same or different.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

Linker: The term “linker” as used herein refers to a connecting peptide between two moieties. For example, a linker can connect an ACE2 moiety to a multimerization moiety.

Multivalent: The term “multivalent” as used herein refers to an ACE2 fusion protein comprising two or more ACE2 moieties, on one, two or more polypeptide chains. The two or more ACE2 moieties can be the same or different.

Octavalent: The term “octavalent” as used herein refers to an ACE2 fusion protein comprising eight ACE2 moieties, whether in the same polypeptide chain or on different polypeptide chains (e.g., on two or four polypeptide chains). The eight ACE2 moieties can be the same or different.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of an ACE2 fusion protein of the disclosure, separate components (e.g., an ACE2 moiety and a multimerization moiety) can be operably linked directly or through peptide linker sequences. In the context of a nucleic acid encoding a fusion protein, such as an ACE2 fusion protein of the disclosure, “operably linked” means that the two nucleic acids are joined such that the amino acid sequences encoded by the two nucleic acids remain in-frame.

Polypeptide, Peptide and Protein: The terms “polypeptide”, “peptide” and “protein” are used interchangeably herein to refer to a polymer of amino acid residues.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tetravalent: The term “tetravalent” as used herein refers to an ACE2 fusion protein comprising four ACE2 moieties, whether in the same polypeptide chain or on two or more polypeptide chains. The four ACE2 moieties can be the same or different.

Treat, Treatment, Treating: As used herein, the terms “treat”, “treatment” and “treating” refer to the reduction or amelioration of the progression, severity and/or duration of a disease or condition and/or the amelioration of one or more symptoms (preferably, one or more discernible symptoms) of a disease or condition resulting from the administration of one or more ACE2 fusion proteins of the disclosure.

In some embodiments, the disease or condition is caused by a coronavirus infection, for example SARS-CoV or SARS-CoV-2, for example COVID-19. In some embodiments, the disease or condition is any other ailment associated with SARS-CoV or SARS-CoV-2 infection, or similar infections. With reference to these diseases and conditions, the terms “treat”, “treatment” and “treating” refer to the reduction or amelioration of the progression, severity and/or duration of the disease or the amelioration of one or more symptoms (preferably, one or more discernible symptoms) of a disease resulting from the administration of one or more ACE2 fusion proteins of the disclosure. In specific embodiments, the terms “treat”, “treatment” and “treating” refer to the amelioration of at least one measurable physical parameter of COVID-19, such as blood oxygen saturation levels, not necessarily discernible by the patient. In other embodiments the terms “treat”, “treatment” and “treating” refer to the inhibition of the progression of COVID-19, either physically by, e.g., stabilization of a discernible symptom, physiologically by, e.g., stabilization of a physical parameter, or both. In other embodiments the terms “treat”, “treatment” and “treating” refer to the reduction or elimination of infection.

### 6.2. ACE2 Fusion Proteins

The present disclosure relates to ACE2 fusion proteins comprising one or more polypeptide chains having the formula [A1]-[L1]-[MM1]-[L2]-[MM2]-[L3]-[A2]-[L4]-[MM3], wherein [A1] represents an ACE2 moiety; [L1] represents a linker; [MM1] represents a multimerization moiety; [L2] represents a linker; [MM2] represents a multimerization moiety; [L3] represents a linker; [A2] represents an ACE2 moiety; [L4] represents a linker; and [MM3] represents a multimerization moiety, and wherein each of [L1], [L2], [L3] and [L4] may be absent, one or two but not all three of [MM1], [MM2] and [MM3] may be absent, and one but not both of [A1] and [A2] may be absent.

In some embodiments, disclosed are ACE2 fusion proteins comprising one or more polypeptide chains having the formula [A1]-[L1]-[MM1]-[L2]-[A2], wherein [A1] represents a first ACE2 moiety; [L1] represents a first linker; [MM1] represents a first multimerization moiety; [L2] represents a second linker; and [A2] represents a second ACE2 moiety, wherein [L1] and [L2] are optional and one of [A1] and [A2] is optional.

In some embodiments, the ACE2 fusion protein comprise one or more polypeptide chains having the formula: [A3]-[L3]-[MM2]-[A1]-[L1]-[MM1]-[L2]-[A2], wherein [A1], [L1], [MM1], [L2], and [A2] are as defined above, [A3] represents a third ACE2 moiety, [L3] represents a third linker, and [MM2] represents a second multimerization moiety, wherein [L3] is optional. Each polypeptide chain having the formula [A3]-[L4]-[MM2]-[A1]-[L1]-[MM1]-[L2]-[A2] may be associated with a polypeptide chain having the formula [A4]-[L4]-[MM3]-[L5]-[A5], wherein [A4] represents a fourth ACE2 moiety, [L4] represents a fourth linker, [MM3] represents a third multimerization moiety that is capable of associating with [MM2], [L5] is a fifth linker, [A5] is a fifth ACE2 moiety, wherein [L4], [L5] and [A5] are optional.

Exemplary multimerization moieties that can be incorporated into the ACE2 fusion proteins as components [MM1], [MM2] and [MM3] are disclosed in Section 6.4. In some embodiments, [MM1] is an Fc domain, for example as described in Section 6.4.1. In some embodiments, [MM1] is a CH1 or CL, e.g., a component of a CH1-CL pair, for example as described in Section 6.4.2.

In some embodiments, [MM2] is an Fc domain, for example as described in Section 6.4.1. In some embodiments, [MM2] is a CH1 or CL, e.g., a component of a CH1-CL pair, for example as described in Section 6.4.2.

In some embodiments, [MM3] is an Fc domain, for example as described in Section 6.4.1. In some embodiments, [MM3] is a CH1 or CL, e.g., a component of a CH1-CL pair, for example as described in Section 6.4.2.

Below are some illustrative ACE2 fusion protein polypeptide chains of the disclosure, presented as formulas in an N-to-C terminal orientation. Individual elements of each are described in detail herein, for example in the subsections that follow and the numbered embodiments.

Formula 1: [A1]-[L1]-[MM1]-[L2]-[MM2]-[L3]-[A2]-[L4]-[MM3], where [A1] and [A2] represent ACE2 moieties, e.g., as defined in any one of numbered embodiments 2 to 23 or 116 to 137, [L1], [L2], [L3], and [L4] represent optional linkers, [MM1] represents a multimerization moiety which is a CH1 domain or a CL domain, e.g., as defined in any one of numbered embodiments 31 to 52, [MM2] represents a multimerization moiety which is or comprises an Fc domain, e.g., as defined in any one of numbered embodiments 76 to 85, and [MM3] represents a multimerization moiety which is a CH1 domain or a CL domain, e.g., as defined in any one of numbered embodiments 145 to 166 (see, e.g., FIG. 2H, inner polypeptide chains). In various embodiments, (a) each of [L1], [L2], [L3], and [L4] is present, e.g., as defined in any one of numbered embodiments 26, 27, 71, 72, 114, 115, 140, or 141; (b) [A1] comprises a PD+ND domain, e.g., as defined in any one of embodiments 7 to 10, optionally having one or more amino acid substitutions as defined in any one of numbered embodiments 12 to 17; (c) [A2] comprises a PD+ND domain, e.g., as defined in any one of embodiments 121 to 124, optionally having one or more amino acid substitutions as defined in any one of numbered embodiments 126 to 131; or (d) any combination of two or all of (a), (b) and (c) (e.g., (a)+(b), (a)+(c), (b)+(c), or (a)+(b)+(c)).

Formula 2: [A1]-[L1]-[MM1], where [A1] represents an ACE2 moiety, e.g., as defined in any one of numbered embodiments 2 to 23, [L1] represents an optional linker, and [MM1] represents a multimerization moiety which is or comprises an Fc domain, e.g., as defined in any one of numbered embodiments 57 to 66 (see, e.g., FIGS. 2B-2D, both polypeptide chains). In various embodiments, (a) [L1] and [L2] are present, e.g., as defined in any one of numbered embodiments 26, 27, 71, or 72, and/or (b) [A1] comprises a PD+ND domain, e.g., as defined in any one of embodiments 7 to 10, optionally having one or more amino acid substitutions as defined in any one of numbered embodiments 12 to 17.

Formula 3: [A1]-[L1]-[MM1]-[L2]-[A2], where [A1] represents an ACE2 moiety, e.g., as defined in any one of numbered embodiments 2 to 23, [L1] represents an optional linker, [MM1] represents a multimerization moiety which is or comprises an Fc domain, e.g., as defined in any one of numbered embodiments 57 to 66, [L2] represents an optional linker, and [A2] represents an ACE2 moiety, e.g., as defined in any one of numbered embodiments 116 to 137 (see, e.g., FIG. 2A and FIG. 2E, both polypeptide chains). In certain embodiments, (a) [L1] and [L2] are present, e.g., as defined in any one of numbered embodiments 26, 27, 71, or 72; (b) [A1] comprises a PD+ND domain, e.g., as defined in any one of embodiments 7 to 10, optionally having one or more amino acid substitutions as defined in any one of numbered embodiments 12 to 17; (c) [A2] comprises a PD domain, e.g., as defined in any one of embodiments 77 to 80, optionally having one or more amino acid substitutions as defined in any one of numbered embodiments 127 to 131 or (d) any combination of two or all of (a), (b) and (c) (e.g., (a)+(b), (a)+(c), (b)+(c), or (a)+(b)+(c)). In various embodiments, (a) [L1] and [L2] are present; (b) [A1] comprises a PD+ND domain, e.g., as defined in any one of embodiments 7 to 10, optionally having one or more amino acid substitutions as defined in any one of numbered embodiments 12 to 17; (c) [A2] comprises a PD+ND domain, optionally having one or more amino acid substitutions as defined in any one of numbered embodiments 127 to 131, e.g., as defined in any one of embodiments 81 to 84; or (d) any combination of two or all of (a), (b) and (c) (e.g., (a)+(b), (a)+(c), (b)+(c), or (a)+(b)+(c)).

Formula 4: [A1]-[L1]-[MM1], where [A1] represents an ACE2 moiety e.g., as defined in any one of numbered embodiments 2 to 23, [L1] represents an optional linker, and [MM1] represents a multimerization moiety which is a CH1 domain or a CL domain, e.g., as defined in any one of numbered embodiments 31 to 52 (see, e.g., FIGS. 2F and 2H-2J, outer polypeptide chains). In various embodiments, (a) [L1] and [L2] are present, e.g., as defined in any one of numbered embodiments 26, 27, 71, or 72, and/or (b) [A1] comprises a PD+ND domain, e.g., as defined in any one of embodiments 7 to 10, optionally having one or more amino acid substitutions as defined in any one of numbered embodiments 12 to 17.

Formula 5: [A1]-[L1]-[MM1]-[L2]-[MM2], where [A1] represents an ACE2 moiety e.g., as defined in any one of numbered embodiments 2 to 23, [L1] represents an optional linker, [MM1] represents a multimerization moiety which is a CH1 domain or a CL domain, e.g., as defined in any one of numbered embodiments 31 to 52, [L2] represents an optional linker, and [MM2] represents a multimerization moiety which comprises an Fc domain, e.g., as defined in any one of numbered embodiments 76 to 85 (see, e.g., FIG. 2J, inner polypeptide chains). In various embodiments, (a) [L1] and [L2] are present, e.g., as defined in any one of numbered embodiments 26, 27, 71, or 72, and/or (b) [A1] comprises a PD+ND domain, e.g., as defined in any one of embodiments 7 to 10, optionally having one or more amino acid substitutions as defined in any one of numbered embodiments 12 to 17.

Formula 6: [A1]-[L1]-[MM1]-[L2]-[MM2]-[L3]-[A2], where [A1] represents an ACE2 moiety e.g., as defined in any one of numbered embodiments 2 to 23, [L1] represents an optional linker, [MM1] represents a multimerization moiety which is a CH1 domain or a CL domain, e.g., as defined in any one of numbered embodiments 31 to 52, [L2] represents an optional linker, [MM2] represents a multimerization moiety which is or comprises an Fc domain, e.g., as defined in any one of numbered embodiments 76 to 85, [L3] represents an optional linker, and [A2] represents an ACE2 moiety, e.g., as defined in any one of numbered embodiments 116 to 137 (see, e.g., FIG. 2F, inner polypeptide chains). In various embodiments, (a) each of [L1], [L2], and [L3] are present, e.g., as defined in any one of numbered embodiments 26, 27, 71, 72, 114, or 115; (b) [A1] comprises a PD+ND domain, e.g., as defined in any one of embodiments 7 to 10, optionally having one or more amino acid substitutions as defined in any one of numbered embodiments 12 to 17; (c) [A2] comprises a PD domain, e.g., as defined in any one of embodiments 77 to 80, optionally having one or more amino acid substitutions as defined in any one of numbered embodiments 126 to 131; or (d) any combination of two or all of (a), (b) and (c) (e.g., (a)+(b), (a)+(c), (b)+(c), or (a)+(b)+(c)).

Formula 7: [MM1]-[L1]-[A1]-[L2]-[MM2], where [MM1] represents a multimerization moiety which is or comprises an Fc domain, e.g., as defined in any one of numbered embodiments 57 to 66, [L1] represents an optional linker, [A1] represents an ACE2 moiety, e.g., as defined in any one of numbered embodiments 2 to 23, [L2] represents an optional linker, and [MM2] represents a multimerization moiety which is a CH1 domain or a CL domain, e.g., as defined in any one of numbered embodiments 31 to 52 (see, e.g., FIG. 2I, inner polypeptide chains). In various embodiments, (a) [L1] and [L2] are present, e.g., as defined in any one of numbered embodiments 26, 27, 71, or 72, and/or (b) [A1] comprises a PD+ND domain, e.g., as defined in any one of embodiments 7 to 10, optionally having one or more amino acid substitutions as defined in any one of numbered embodiments 12 to 17.

Formula 8: [A1]-[L1]-[MM1]-[L2]-[A2], where [A1] represents an ACE2 moiety e.g., as defined in any one of numbered embodiments 2 to 23, [L1] represents an optional linker, [MM1] represents a multimerization moiety which is a CH1 domain or a CL domain, e.g., as defined in any one of numbered embodiments 31 to 52, [L2] represents an optional linker, and [A2] represents an ACE2 moiety, e.g., as defined in any one of numbered embodiments 116 to 137 (see, e.g., FIG. 2G, outer polypeptide chains). In various embodiments, (a) [L1] and [L2] are present; (b) [A1] comprises a PD+ND domain, e.g., as defined in any one of embodiments 7 to 10, optionally having one or more amino acid substitutions as defined in any one of numbered embodiments 12 to 17; (c) [A2] comprises a PD domain, e.g., as defined in any one of embodiments 77 to 80, optionally having one or more amino acid substitutions as defined in any one of numbered embodiments 127 to 131; or (d) any combination of two or all of (a), (b) and (c) (e.g., (a)+(b), (a)+(c), (b)+(c), or (a)+(b)+(c)).

An ACE2 fusion protein of the present disclosure may comprise one or more polypeptide chains of Formulas 1-8 above. Exemplary ACE2 fusion proteins are depicted in FIGS. 2A-2J. Exemplary ACE2 fusion proteins disclosed herein include:
(1) An ACE2 fusion protein comprising two polypeptides of Formula 3 associated with one another via Fc pairing (see, e.g., FIG. 2A and FIG. 2E)
(2) An ACE2 fusion protein comprising two polypeptides of Formula 2 associated with one another via Fc pairing (see, e.g., FIGS. 2B-2D).
(3) An ACE2 fusion protein comprising two polypeptides of Formula 6 associated with one another via Fc pairing, each associated with a polypeptide of Formula 4 via CH1-CL pairing (see, e.g., FIG. 2F)
(4) An ACE2 fusion protein comprising two polypeptides of Formula 6 associated with one another via Fc pairing, each associated with a polypeptide of Formula 8 via CH1-CL pairing (see, e.g., FIG. 2G)
(5) An ACE2 fusion protein comprising two polypeptides of Formula 1 associated with one another via Fc pairing, each associated with two polypeptides of Formula 4 via CH1-CL pairing (see, e.g., FIG. 2H)
(6) An ACE2 fusion protein comprising two polypeptides of Formula 7 associated with one another via Fc pairing, each associated with a polypeptide of Formula 4 via CH1-CL pairing (see, e.g., FIG. 2I)
(7) An ACE2 fusion protein comprising two polypeptides of Formula 5 associated with one another via Fc pairing, each associated with a polypeptide of Formula 4 via CH1-CL pairing (see, e.g., FIG. 2J)

Exemplary ACE2 moieties that can be incorporated into the ACE2 fusion proteins as component(s) [A1] and/or [A2] and/or [A3] and/or [A4] and/or [A5] are disclosed in Section 6.3.

Exemplary linkers that can be incorporated into the ACE2 fusion proteins as component(s) [L1] and/or [L2] and/or [L3] and/or [L4] and/or [L5] are disclosed in Section 6.5.

### 6.3. ACE2 Moiety

SARS-CoV-2 docks on a host cell's extracellular surface by binding to ACE2, an enzyme expressed on a variety of cells, including respiratory epithelia.

The amino acid sequence for human ACE2 is assigned the NCBI reference sequence NP_001358344.1 and the UniProtKB accession number Q9BYF1, reproduced below with the signal peptide underlined.

(SEQ ID NO: 1)
MSSSSWLLLS LVAVTAAQST IEEQAKTFLD KFNHEAEDLF
YQSSLASWNY NTNITEENVQ NMNNAGDKWS AFLKEQSTLA
QMYPLQEIQN LTVKLQLQAL QQNGSSVLSE DKSKRLNTIL
NTMSTIYSTG KVCNPDNPQE CLLLEPGLNE IMANSLDYNE
RLWAWESWRS EVGKQLRPLY EEYVVLKNEM ARANHYEDYG
DYWRGDYEVN GVDGYDYSRG QLIEDVEHTF EEIKPLYEHL
HAYVRAKLMN AYPSYISPIG CLPAHLLGDM WGRFWTNLYS
LTVPFGQKPN IDVTDAMVDQ AWDAQRIFKE AEKFFVSVGL
PNMTQGFWEN SMLTDPGNVQ KAVCHPTAWD LGKGDFRILM
CTKVTMDDFL TAHHEMGHIQ YDMAYAAQPF LLRNGANEGF
HEAVGEIMSL SAATPKHLKS IGLLSPDFQE DNETEINFLL
KQALTIVGTL PFTYMLEKWR WMVFKGEIPK DQWMKKWWEM
KREIVGVVEP VPHDETYCDP ASLFHVSNDY SFIRYYTRTL
YQFQFQEALC QAAKHEGPLH KCDISNSTEA GQKLFNMLRL
GKSEPWTLAL ENVVGAKNMN VRPLLNYFEP LFTWLKDQNK
NSFVGWSTDW SPYADQSIKV RISLKSALGD KAYEWNDNEM
YLFRSSVAYA MRQYFLKVKN QMILFGEEDV RVANLKPRIS
FNFFVTAPKN VSDIIPRTEV EKAIRMSRSR INDAFRLNDN
SLEFLGIQPT LGPPNQPPVS IWLIVFGVVM GVIVVGIVIL
IFTGIRDRKK KNKARSGENP YASIDISKGE NNPGFQNTDD
VQTSF

Under normal circumstances, ACE2 contributes to the regulation of vascular tone and blood pressure by cleaving angiotensin precursors, which it achieves via its peptidase domain (PD). The ACE2-PD is the largest domain of ACE2, corresponding to amino acids 18 to 615, the sequence of which is reproduced below.

(SEQ ID NO: 2)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYAD

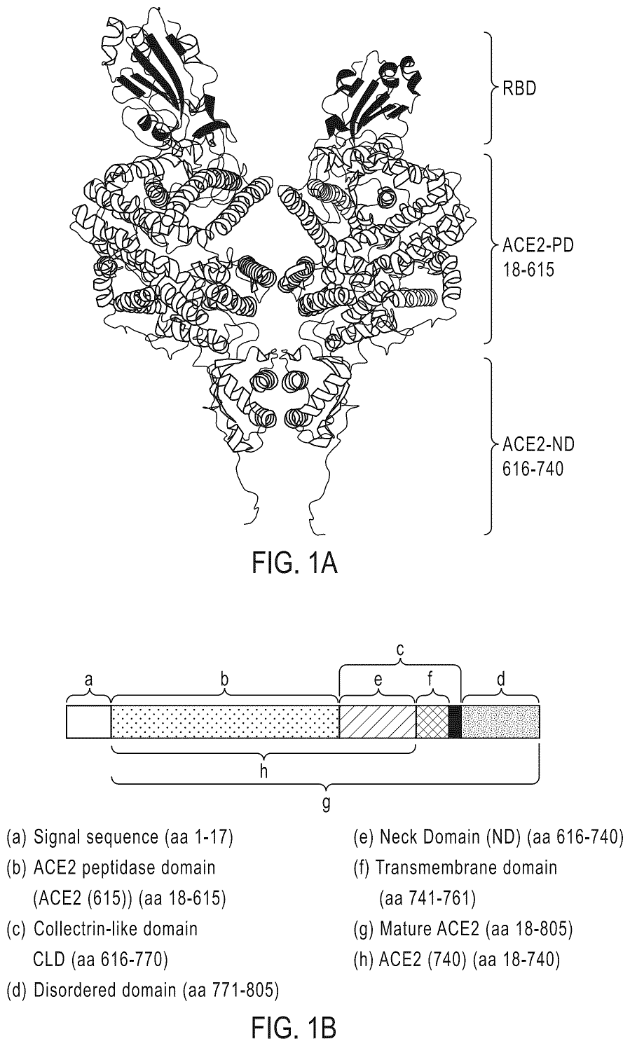

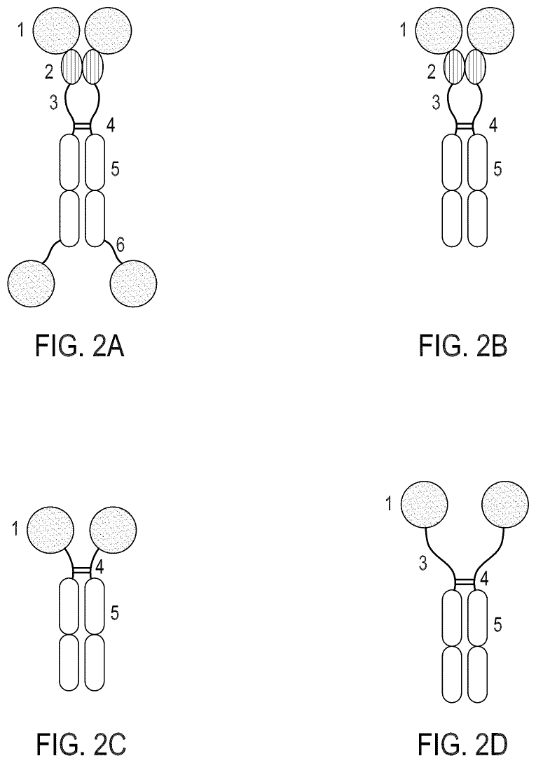

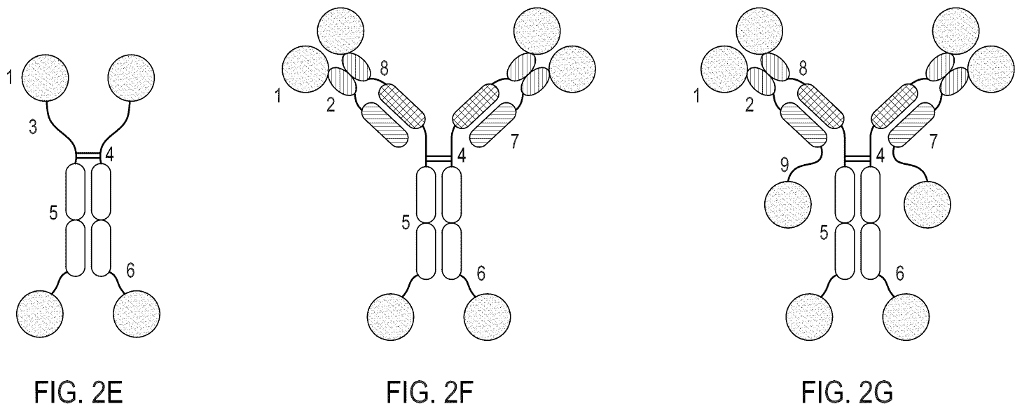

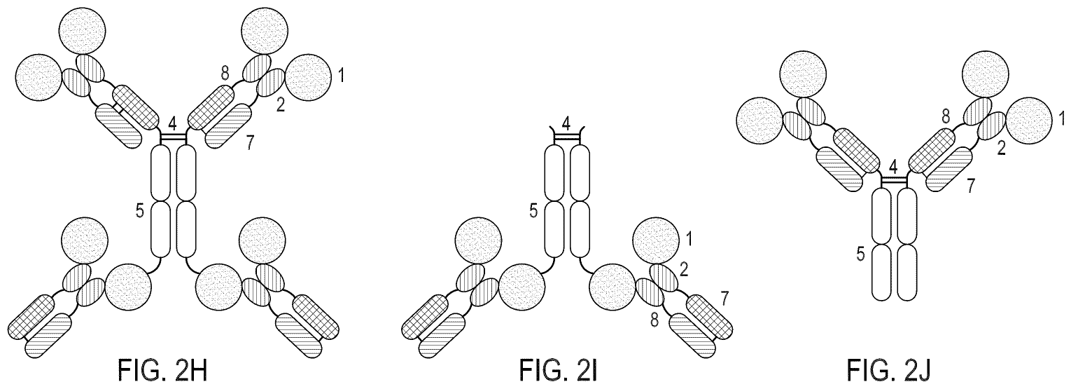

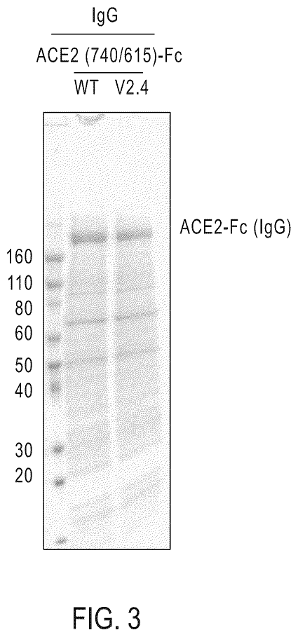

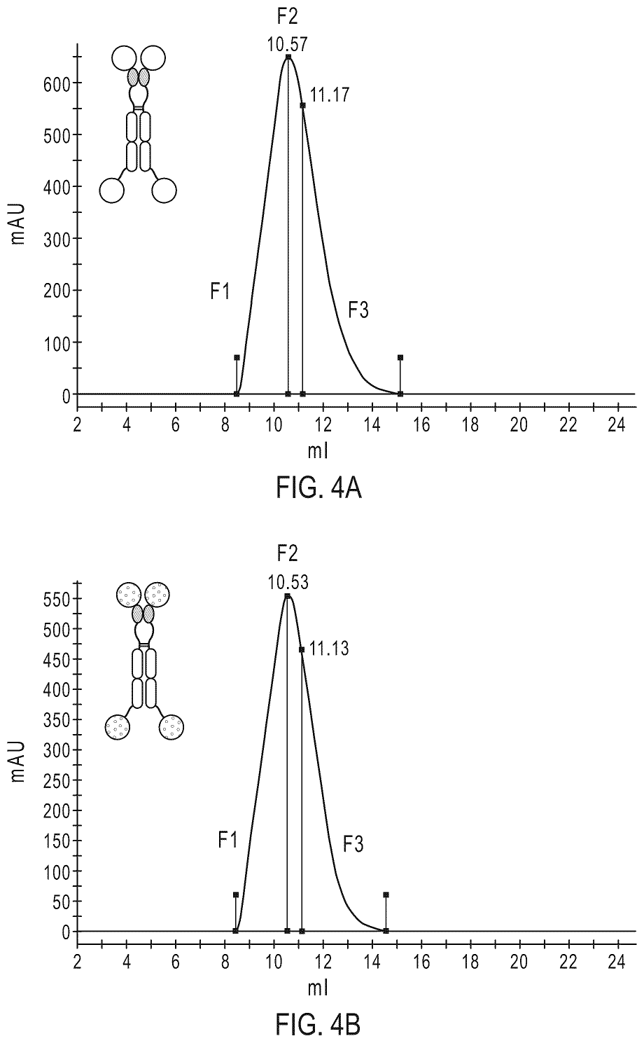

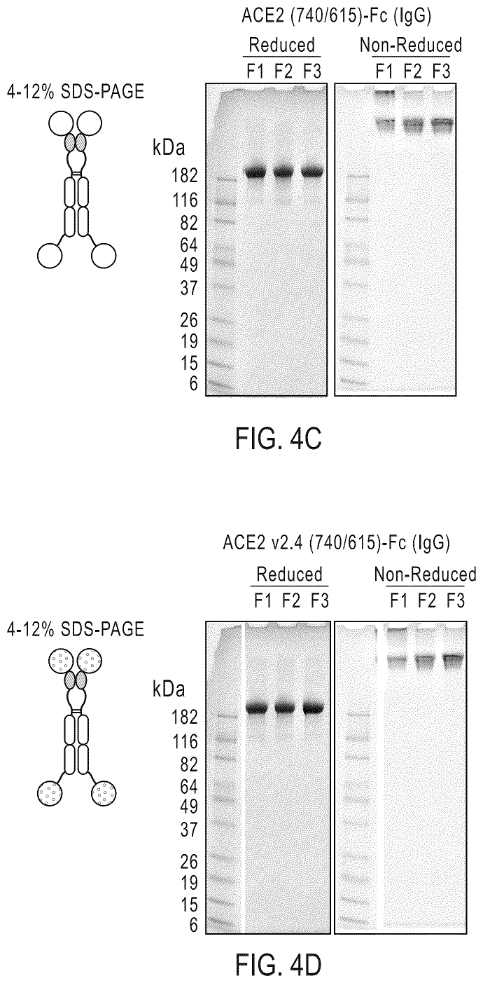

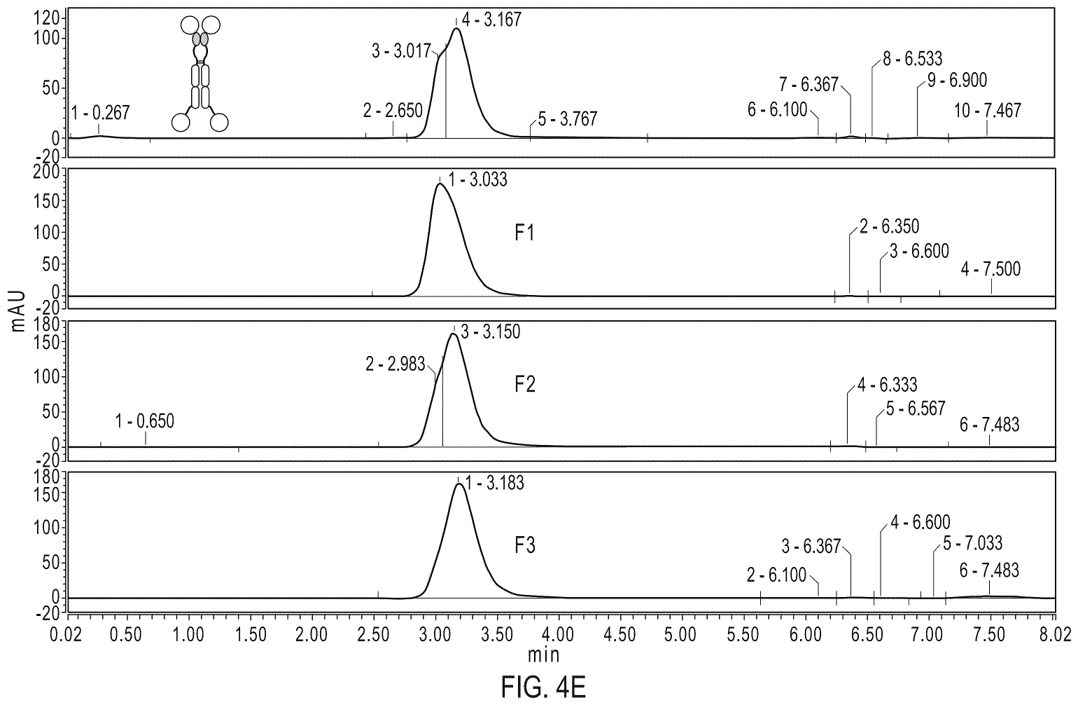

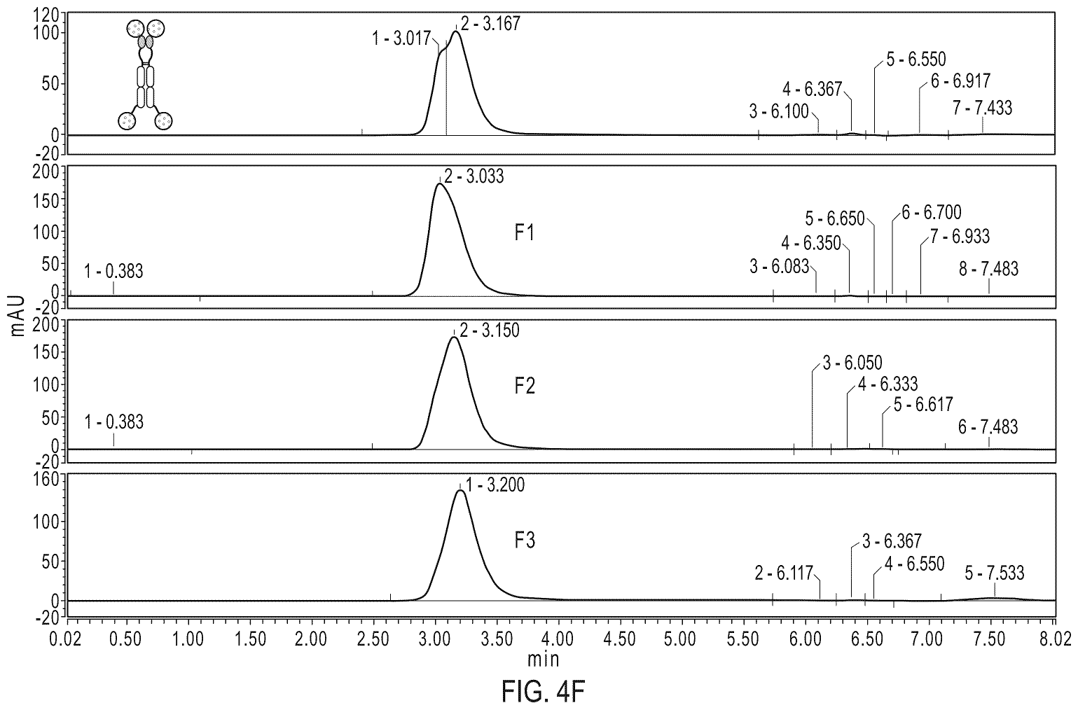

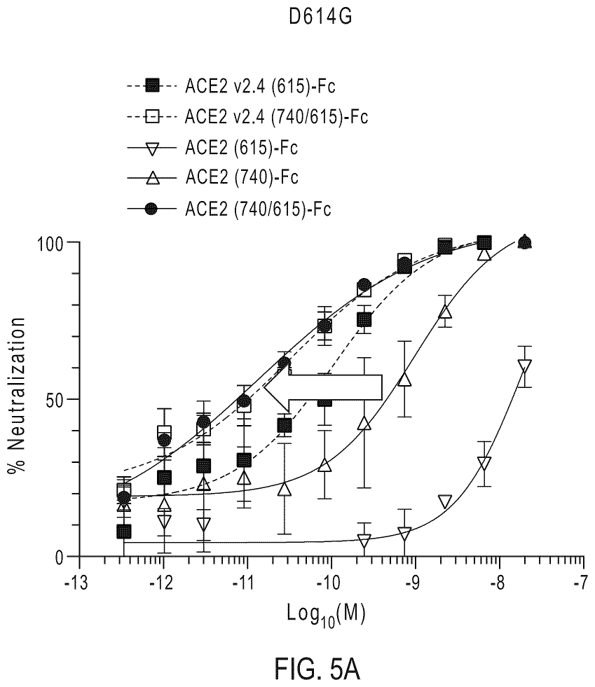

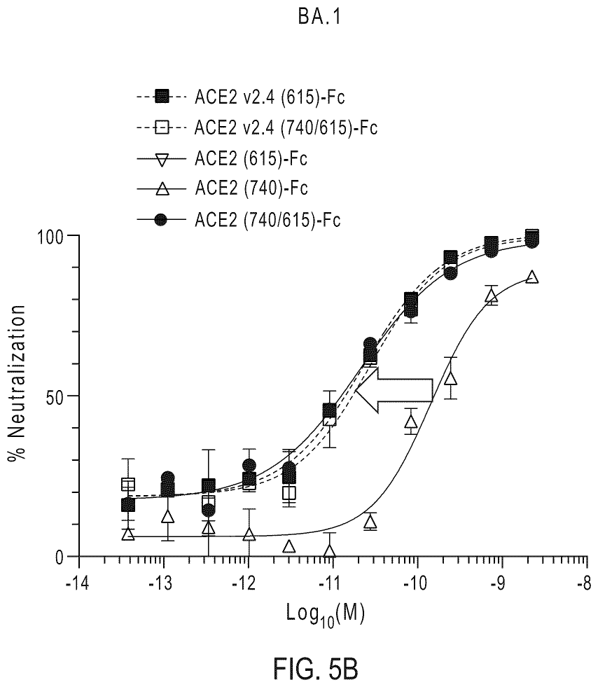

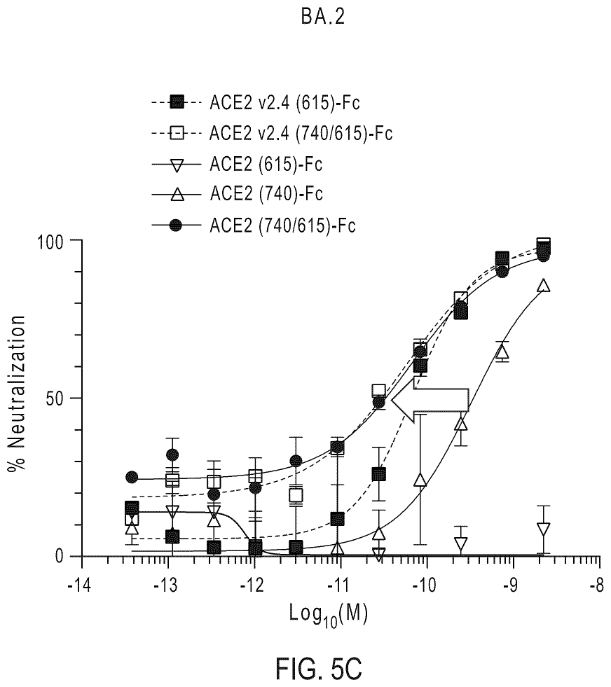

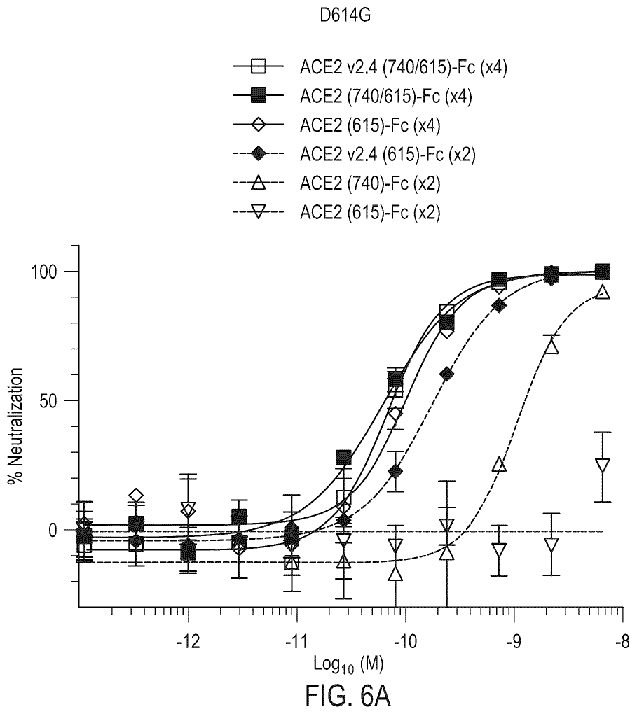

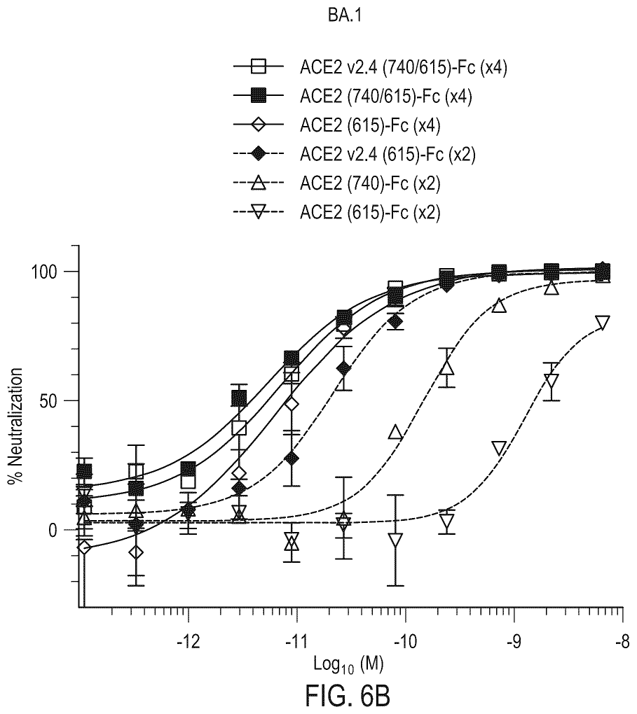

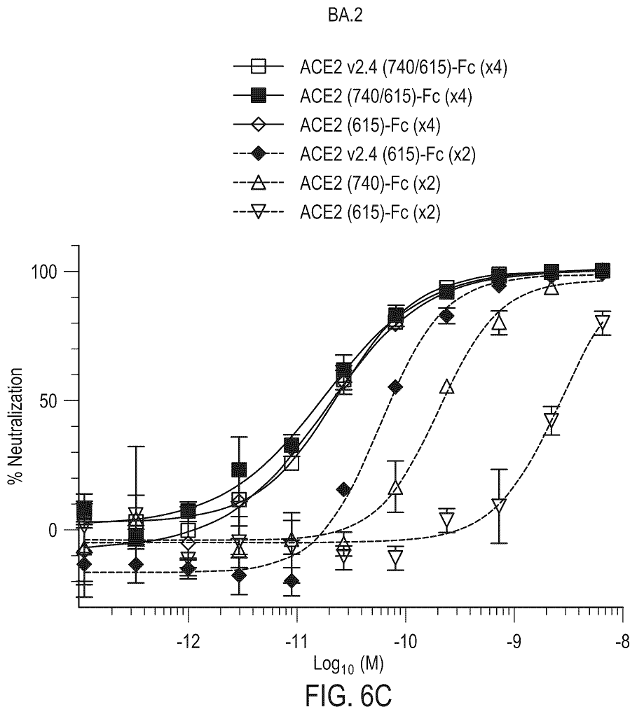

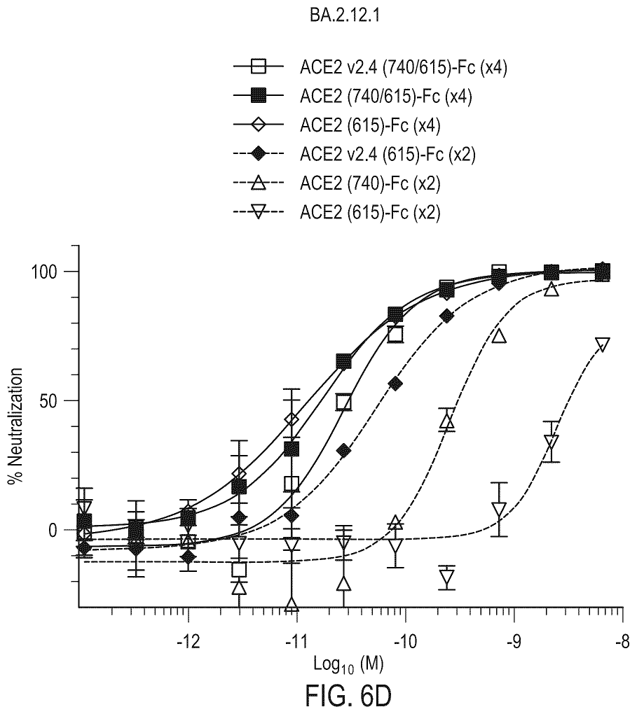

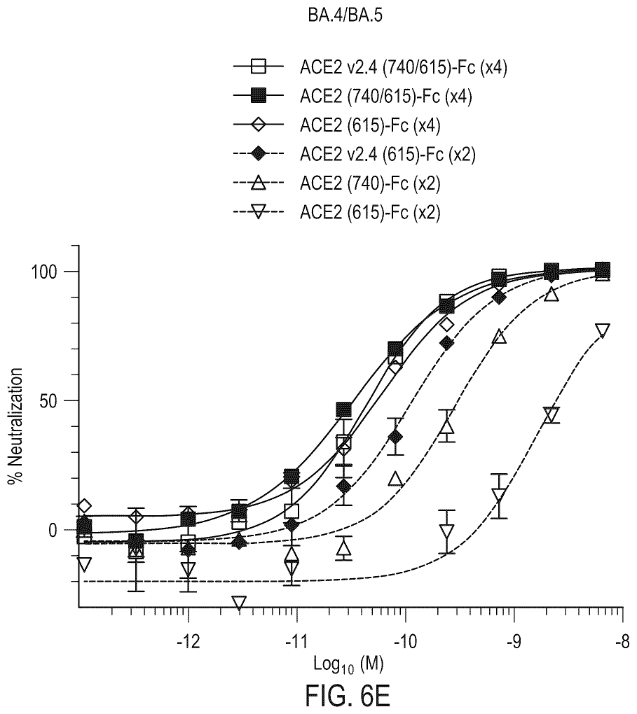

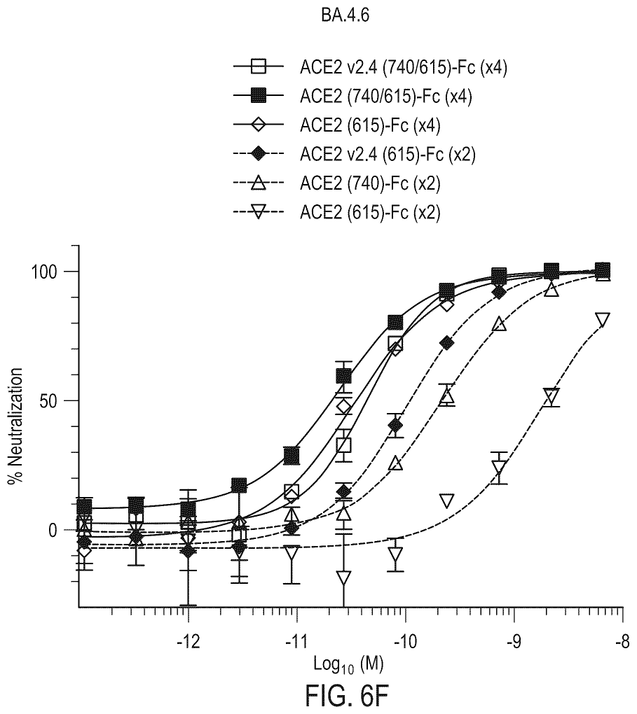

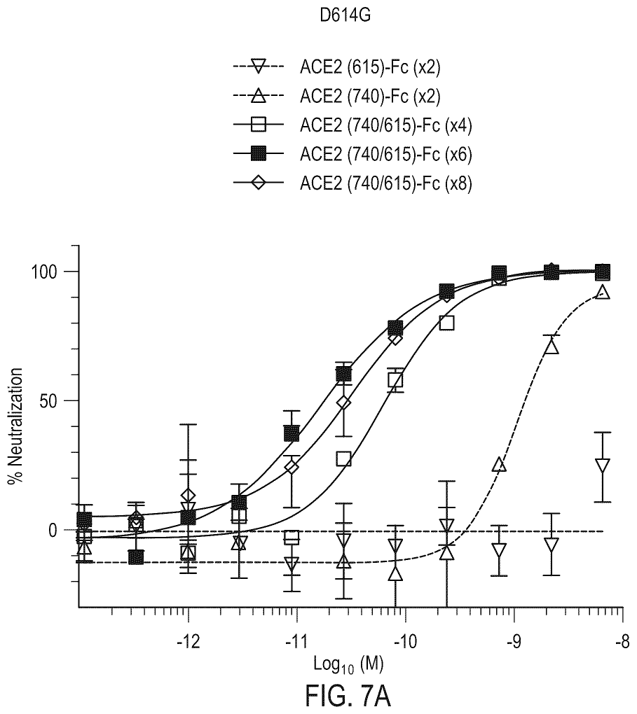

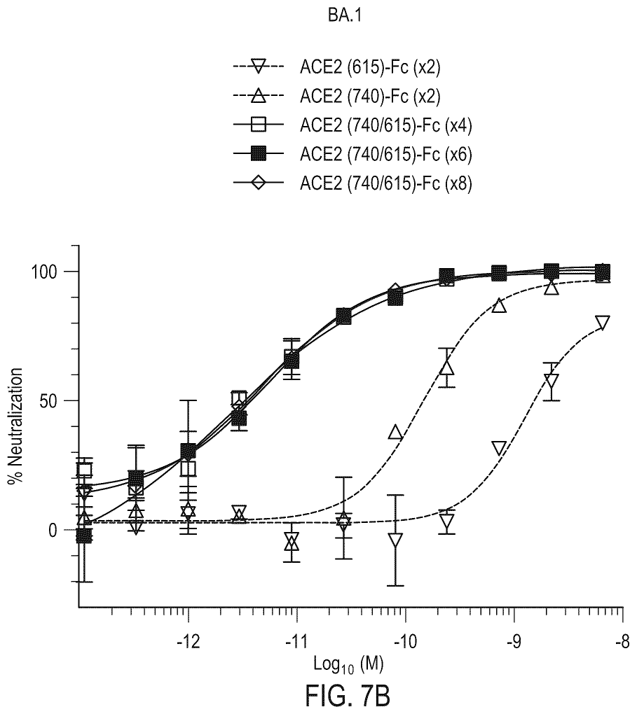

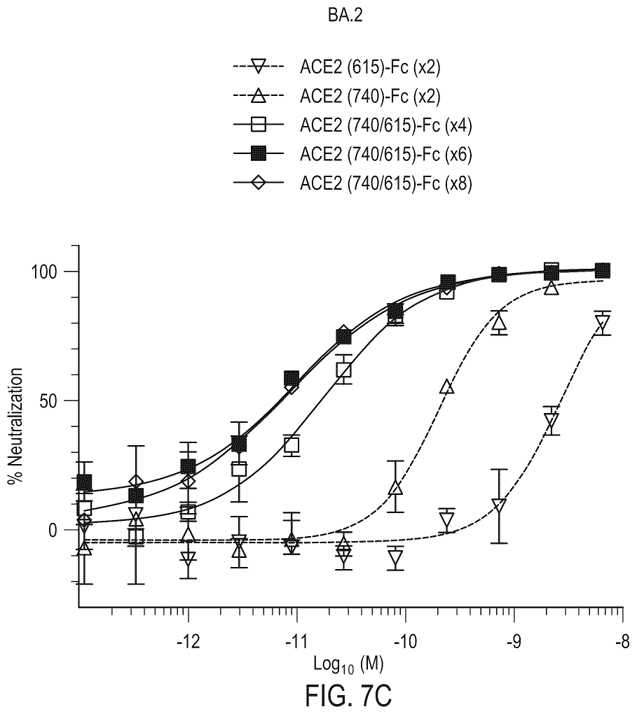

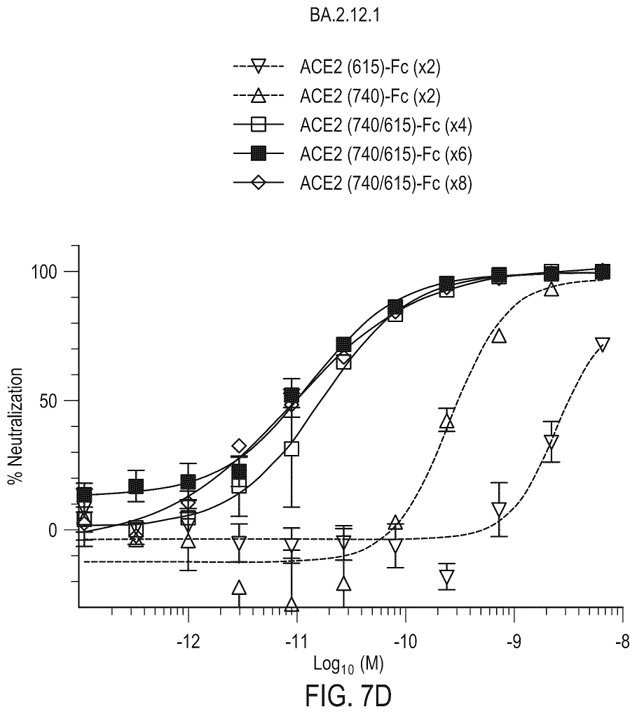

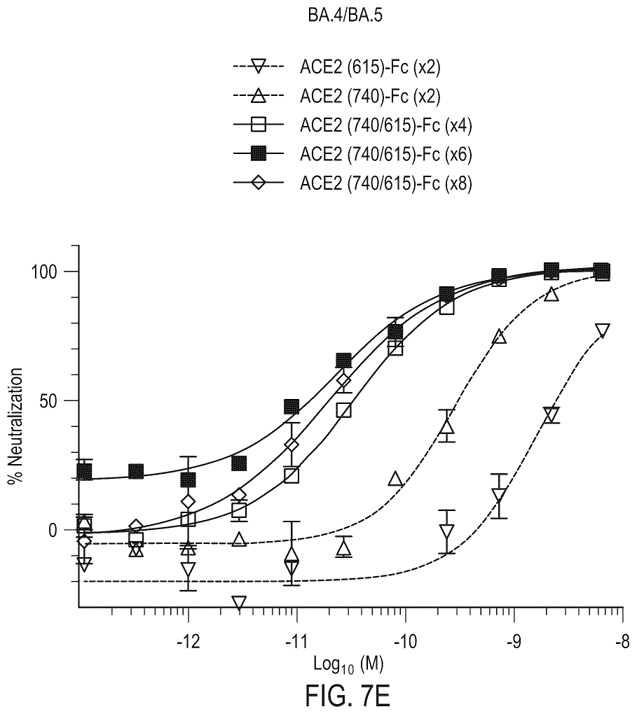

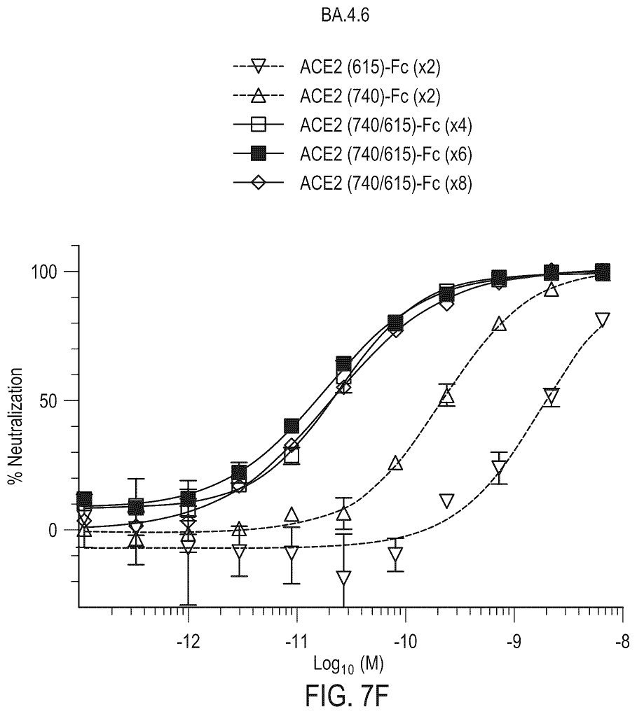

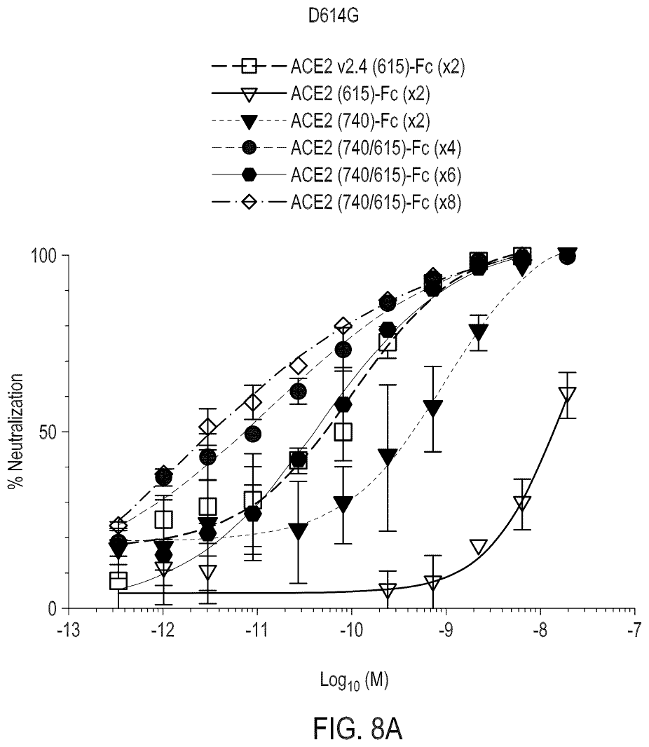

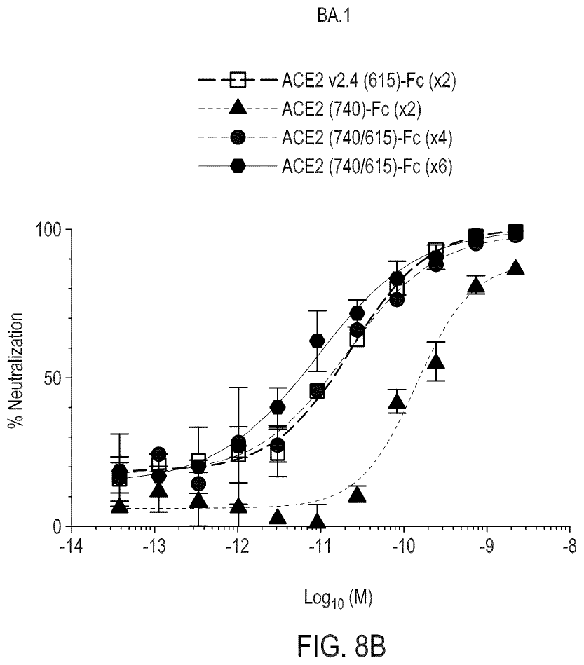

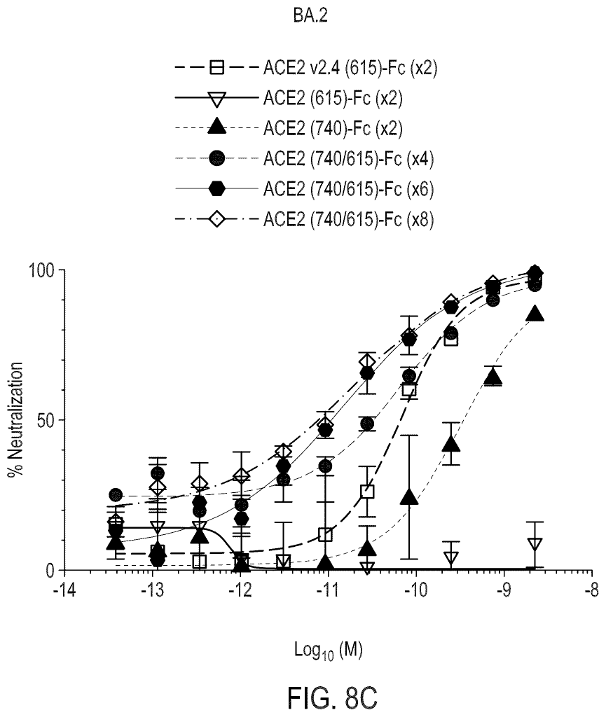

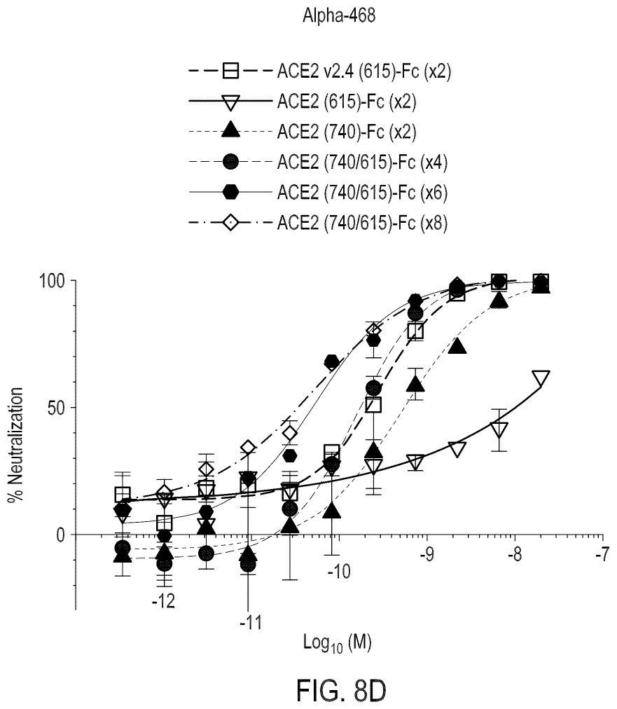

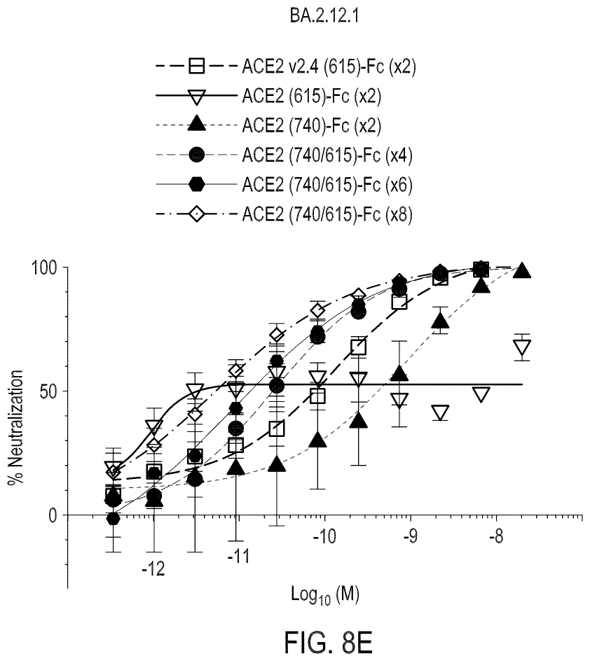

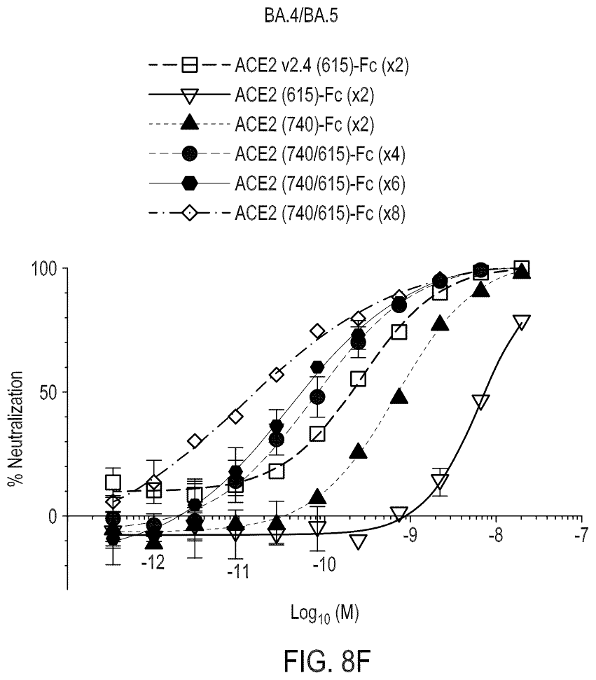

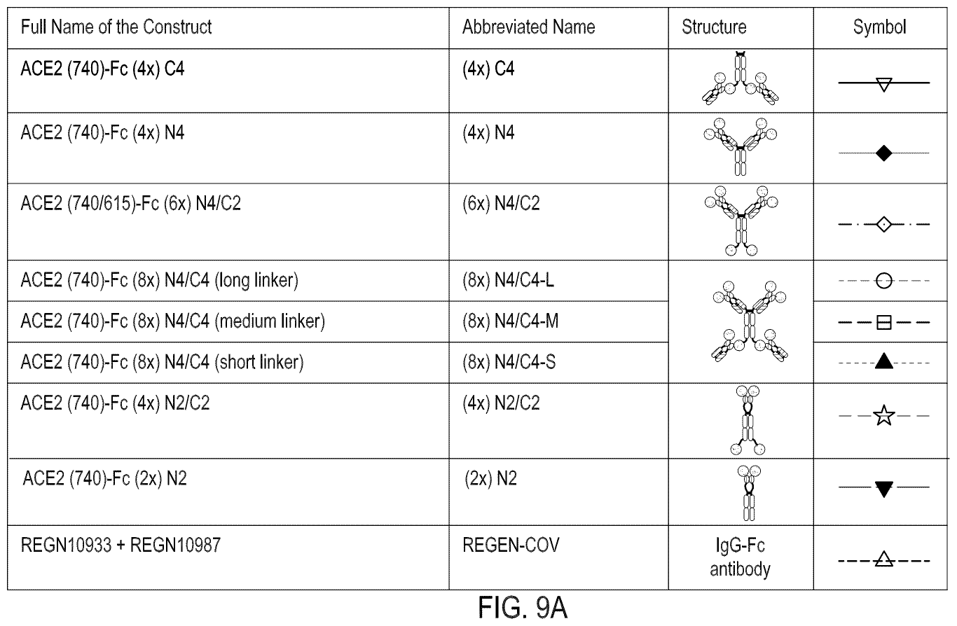

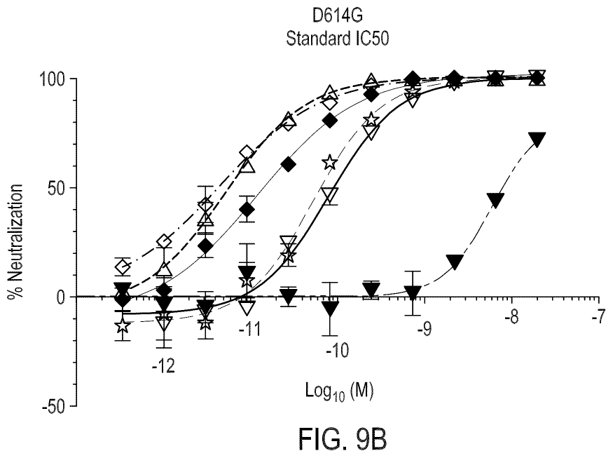

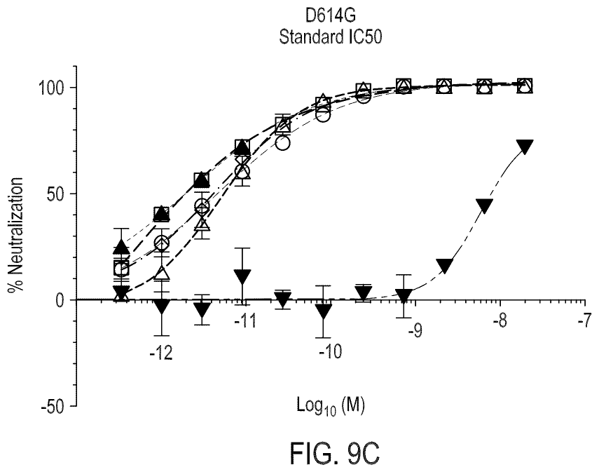

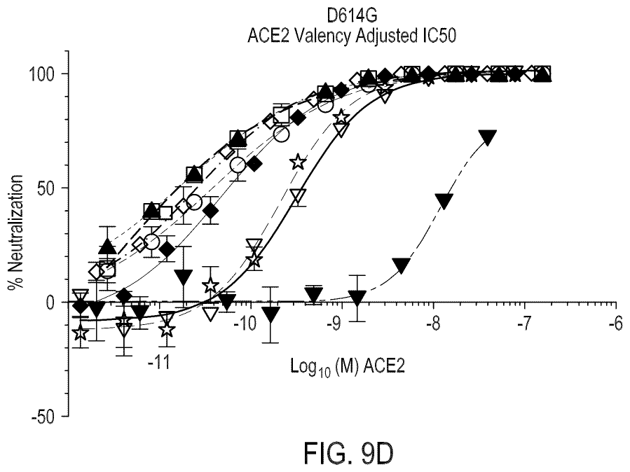

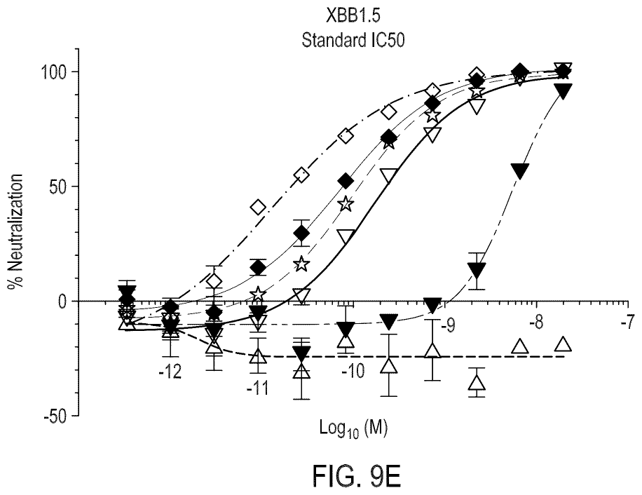

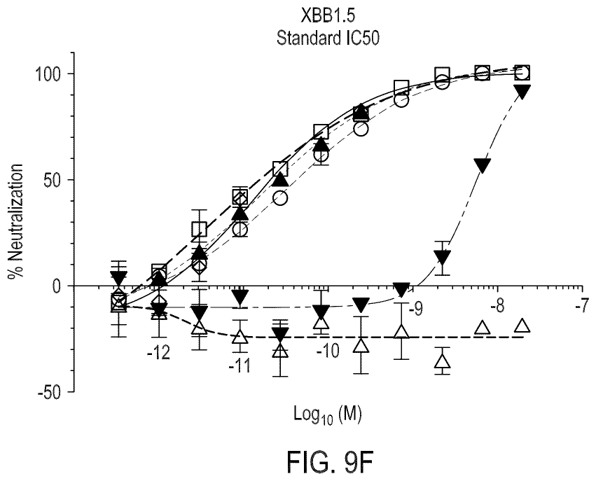

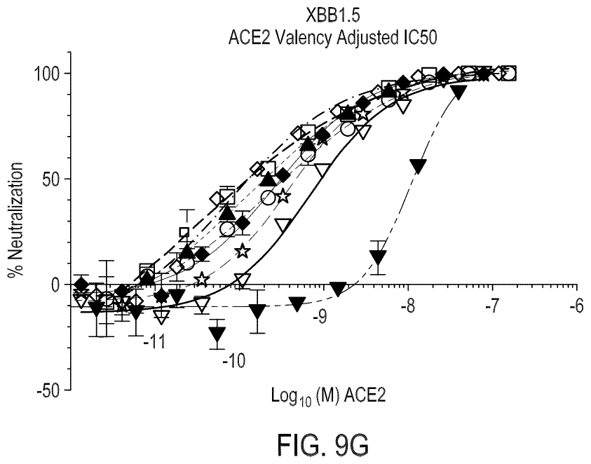

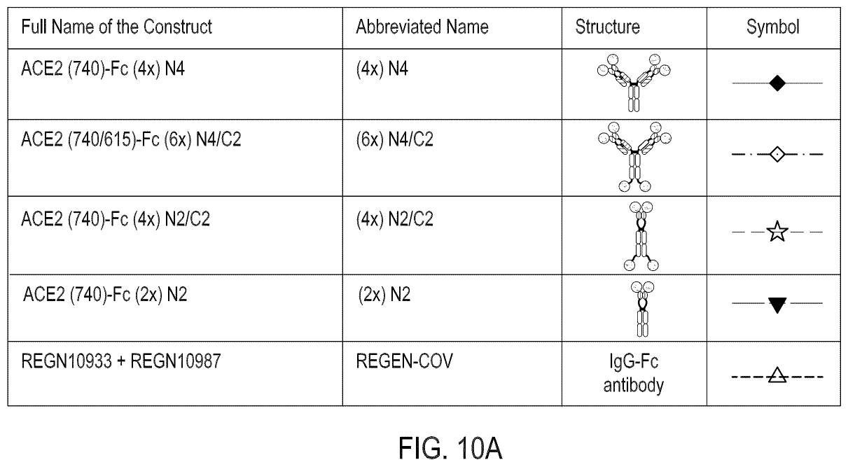

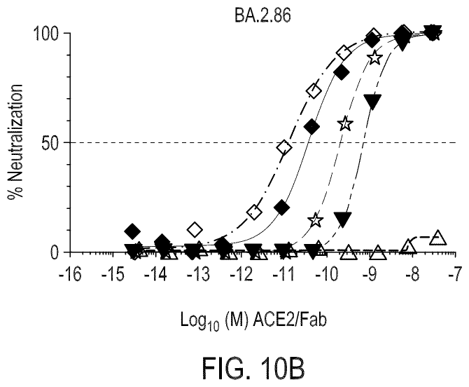

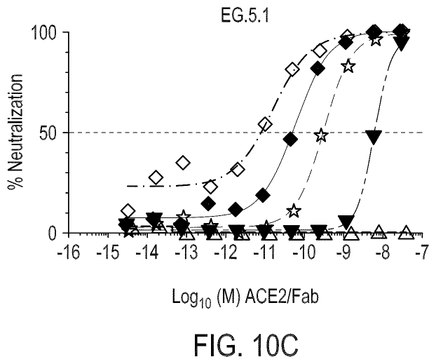

The other domain of ACE2 is its collectrin-like domain (CLD; aa 616-770), which contains an extracellular neck domain (ND; aa 616-740) that facilitates dimerization, and a single transmembrane domain (TM; aa 741-761) (FIGS. 1A and 1B).

The extracellular portion of ACE2 consists of the PD and ND (ACE2 (740); aa 18-740), the amino acid sequence of which is reproduced below.

(SEQ ID NO: 3)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS

SARS-CoV or SARS-CoV-2 interaction with ACE2 involves large viral protrusions called spike (S) proteins. The S protein of SARS-CoV or SARS-CoV-2 consists of two subunits: S1 and S2. The receptor binding domain (RBD) of S1 is responsible for binding the ACE2-PD via polar interactions (FIG. 1A). More specifically, an extended loop of RBD spans over the a1 helix of ACE2-PD like a bridge, yet it also interacts with the a2 helix and the loop that connects the B3 and 4 strands of ACE2-PD (Yan et al., 2020, Science. 367:1444-1448). An exemplary SARS-CoV RBD sequence is reproduced below.

(SEQ ID NO: 4)
TNLCPFGEVFNATKFPSVYAWERKKISNCVADYSVLYNSTFFSTFKCYGV
SATKLNDLCFSNVYADSFVVKGDDVRQIAPGQTGVIADYNYKLPDDDMGC
VLAWNTRNIDATSTGNYNYKYRYLRHGKLRPFERDISNVPFSPDGKPCTP
PALNCYWPLNDYGFYTTTGIGYQPYRVVVLSFELLNAPATVCGPK

An exemplary SARS-CoV-2 RBD sequence is reproduced below.

(SEQ ID NO: 5)
TNLCPFGEVFNATRFASVYAWNRKRISNCVADYSVLYNSASFSTFKCYGV
SPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNG
VEGFNCYFPLQSYGFQPTNGVGYQPYRVVVLSFELLHAPATVCGPK

The ACE2 fusion proteins of the disclosure comprise an ACE2 moiety that has an amino acid sequence with at least 70% sequence identity to an extracellular portion of human ACE2 that is capable of binding the RBD of SARS-CoV or SARS-CoV-2 RBD, for example an amino acid sequence having at least 70% sequence identity to the peptidase domain (PD) of human ACE2.

The binding affinity of an ACE2 moiety to RBD peptides can be assessed using various binding assays. For instance, biolayer interferometry (BLI) can be used to measure binding of free RBD to an immobilized ACE2 or a free ACE2 to an immobilized RBD by analyzing the reflection patterns of light from the sensor surface. BLI and other binding affinity assays can be used to determine the effect of ACE2 mutations on its affinity to RBD.

Several affinity-enhancing mutations of ACE2 have been reported. For instance, hydrophobic substitutions of T27 of ACE2 increases hydrophobic packing with aromatic residues of the S protein, whereas D30E mutation allows interaction with K417 of the S protein (Yan et al., 2020, Science. 367:1444-1448). Moreover, a slew of single amino acid substitutions that enhance ACE2 binding affinity to RBD have been characterized (Chan et al., 2020, Science. 369:1261-1265; Laurini et al., 2021. ACS Nano 15(4):6929-6948), which may be utilized to generate ACE2 moieties with enhanced binding affinity to an RBD, e.g., a SARS-CoV-2 RBD.

In certain aspects, the ACE2 moiety has an amino acid sequence that is at least 70%, at least 80% or at least 90% identical to the PD of human ACE2, corresponding to amino acids 18 to 615 (SEQ ID NO:2), and in various embodiments has an amino acid sequence that is at least about 91%, at least about 92%, at least about 93%, at least about 94%, at least about 95%, at least about 96%, at least about 97%, at least about 98%, at least about 99%, or 100% identical to the PD of human ACE2.

In further aspects, the ACE2 moiety has an amino acid sequence that is at least 70%, at least 80% or at least 90% identical to the PD+ND of human ACE2, corresponding to amino acids 18 to 740 (SEQ ID NO:3), and in various embodiments has an amino acid sequence that is at about 91%, at least about 92%, at least about 93%, at least about 94%, at least about 95%, at least about 96%, at least about 97%, at least about 98%, at least about 99%, or 100% identical to the PD+ND of human ACE2.

In some embodiments, the PD portion of an ACE2 moiety can include one or more amino acid substitutions that increase binding affinity to an RBD, e.g., a SARS-CoV2 RBD. These substitutions may involve the amino acids 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 357, 386, 393, or 519 of full length human ACE2 (SEQ ID NO: 1), for example, one or more of the amino acid substitutions listed in Table 1. Unless indicated otherwise, all position numbering is in relation to full length human ACE2 (SEQ ID NO: 1).

TABLE 1
ACE2-PD Amino Acid Substitutions Enhancing S Protein Binding Affinity

| | | | | | | | |
|---|---|---|---|---|---|---|---|
| S19P | E23F | Q24T | A25V | K26I | T27M | L29F | D30I |
| | | | | K26A | T27L | | D30E |
| | | | | K26D | T27A | | |
| | | | | | T27D | | |
| | | | | | T27K | | |
| | | | | | T27H | | |
| | | | | | T27W | | |
| | | | | | T27Y | | |
| | | | | | T27F | | |
| | | | | | T27C | | |
| K31W | N33D | H34V | E35V | L39K | F40D | Y41R | Q42M |
| K31Y | | H34S | E35C | L39R | F40R | | Q42L |
| | | H34P | E35D | | | | Q42I |
| | | H34K | | | | | Q42V |
| | | | | | | | Q42K |
| | | | | | | | Q42C |
| A65W | W69I | F72Y | E75A | Q76M | L79I | M82C | Q89I |
| | W69V | | E75S | Q76I | L79V | | Q89D |
| | W69T | | E75T | Q76V | L79T | | Q89P |
| | W69K | | E75K | Q76T | L79W | | |
| | | | E75R | Q76R | L79Y | | |
| | | | E75W | Q76Y | L79F | | |
| | | | E75G | | L79P | | |
| N90M | L91P | T92M | T324E | Q325P | N330L | L351F | R357K |
| N90L | | T92L | T324P | | N330H | | |
| N90I | | T92I | | | N330W | | |
| N90V | | T92V | | | N330Y | | |
| N90A | | T92A | | | N330F | | |
| N90S | | T92N | | | | | |
| N90T | | T92Q | | | | | |
| N90Q | | T92D | | | | | |
| N90D | | T92E | | | | | |
| N90E | | T92K | | | | | |
| N90K | | T92R | | | | | |
| N90R | | T92H | | | | | |
| N90H | | T92W | | | | | |
| N90W | | T92Y | | | | | |
| N90Y | | T92F | | | | | |
| N90F | | T92P | | | | | |
| N90P | | T92G | | | | | |
| N90G | | T92C | | | | | |
| N90C | | | | | | | |
| A386L | R393K | R518G | | | | | |
| A386D | | | | | | | |

In some embodiments, the PD portion of the ACE2 moiety includes a combination of two or more amino acid substitutions that enhance its affinity to an RBD, e.g., a SARS-CoV-2 RBD as compared to the corresponding wildtype sequence. In certain specific embodiments, The PD portion of the ACE2 moiety comprises two, three, four, five, six or more of the substitutions listed in Table 1 as compared to full length human ACE2 (SEQ ID NO:1).

In some embodiments, the ACE2 moiety comprises one or more amino acid substitutions associated with high levels of enhanced binding to an RBD, e.g., a SARS-CoV-2 RBD. For instance, these amino acid substitution combinations can include one or more substitutions at the amino acids 25, 27, 31, 34, 42, 79, 90, 92, 324, 325, 330, and 386 of human ACE2 (SEQ ID NO:1), for example one or more of the substitutions sets forth in Table 1, which are associated with the highest binding affinity increases to the SARS-CoV-2 RBD.

In certain aspects, the ACE moiety can include combinations of amino acid substitutions that have been shown to be associated with increased RBD affinity. For instance, one such example is ACE2 v2.4, which combines the amino acid substitutions T27Y, L79T, and N330Y (Chan et al., 2020, Science. 369:1261-1265). Accordingly, in certain embodiments, the combinations of amino acid substitutions of the ACE2 moiety can include the amino acid substitutions T27Y, L79T, and N330Y, optionally with one or more additional substitutions. In some embodiments, the ACE2 moiety comprises the PD of ACE2 (e.g., an amino acid sequence having the sequence of SEQ ID NO:2) with the amino acid substitutions T27Y, L79T, and N330Y (e.g., the ACE2 moiety designated herein as ACE2-PD v2.4). The amino acid sequence of ACE2-PD v2.4 is reproduced below.

(SEQ ID NO: 11)
QSTIEEQAKYFLDKFNHEAEDLFYQSSLASWNYNTNITEENVQNMNNAGD
KWSAFLKEQSTTAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWEYSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYAD

In further embodiments, the ACE2 moiety comprises the PD+ND of ACE2 (e.g., an amino acid sequence having the sequence of SEQ ID NO:3) with the amino acid substitutions T27Y, L79T, and N330Y (e.g., the ACE2 moiety designated herein as ACE2-PD+ND v2.4). The amino acid sequence of ACE2-PD+ND v2.4 is reproduced below.

(SEQ ID NO: 12)
QSTIEEQAKYFLDKENHEAEDLFYQSSLASWNYNTNITEENVQNMNNAGD
KWSAFLKEQSTTAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWEYSMLTDPGNVQKAVCHPTAWDLGKGDFRILMCTKVTMD
DELTAHHEMGHIQYDMAYAAQPFLLRNGANEGFHEAVGEIMSLSAATPKH
LKSIGLLSPDFQEDNETEINFLLKQALTIVGTLPFTYMLEKWRWMVEKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS

In addition to affinity-enhancing mutations, several mutations of ACE2 have been reported which reduce or eliminate the catalytic activity of ACE2. For instance, Zn2+-coordinating residues, H374 and H378, are important for catalytic activity, and their substitution with asparagine (N) residues results in a catalytically inactive ACE2 (Moore et al., 2004, J Virol. 78(19): 10628-35). In addition, the H435L mutation has also been shown to inactivate ACE2 catalytic activity (Glasgow et al., 2020, Proc Natl Acad Sci USA. 117(45):28046-55).

In some embodiments, the PD portion of an ACE2 moiety can include one or more amino acid substitutions that decrease catalytic activity of ACE2. These substitutions may involve the amino acids 374, 378, and/or 435 of human ACE2, for example, H374N, H378N and/or H435L. In some embodiments, the PD portion of the ACE2 moiety includes the amino acid substitutions H374N and H378N. In some embodiments, the PD portion of the ACE2 moiety includes the amino acid substitution H435L.

The catalytic (monocarboxypeptidase) activity of an ACE2 moiety can be assessed using various activity assays. For instance, the cleavage of phenylalanine on Ang II and Ap-13 substrates may be measured using fluorometric-based techniques (Liu et al., 2020, Int J Biol Macromol. 165(Pt B): 1626-33). This or other activity assays can be used to determine the effect of ACE2 mutations on its catalytic activity.

In certain specific embodiments, the ACE2 moiety:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of the ACE2 amino acid sequence of SEQ ID NO:1;
(d) comprises at least one amino acid substitution set forth in Table 1;
(e) comprises the amino acid substitutions T27Y, L79T, and N330Y as compared to the ACE2 amino acid sequence of SEQ ID NO: 1;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

In further certain specific embodiments, the ACE2 moiety:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of the ACE2 amino acid sequence of SEQ ID NO:1;
(d) comprises at least one amino acid substitution set forth in Table 1;
(e) comprises the amino acid substitutions T27Y, L79T, and N330Y as compared to the ACE2 amino acid sequence of SEQ ID NO:1;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1);
(g) comprises at least one amino acid substitution that decreases catalytic activity; or
(h) any combination of two, three or more of (a)-(g).

### 6.4. Multimerization Moiety

In some embodiments, the ACE2 fusion proteins of the disclosure include one or more multimerization moieties, for example one or more multimerization moieties that are or comprise an Fc domain. In certain embodiments, an ACE2 fusion protein of the disclosure comprises a single multimerization moiety (e.g., a single Fc domain) and/or an ACE2 fusion protein of the disclosure comprises two or more multimerization moieties (e.g., two or more Fc domains that can associate to form an Fc region and/or a one or more CH1-CL domain pairs). In some embodiments, the ACE2 fusion protein is a dimer and the Fc region comprises two IgG-derived Fc domains, for example as described in Section 6.4.1 below.

### 6.4.1. Fc Domains

The ACE2 fusion proteins of the disclosure can include an Fc domain, or a pair of Fc domains that associate to form an Fc region, derived from any suitable species operably linked to an ACE2 moiety. In one embodiment the Fc domain is derived from a human Fc domain. In preferred embodiments, the ACE2 moiety is fused to an IgG Fc domain.

The Fc domains that can be incorporated into ACE2 fusion proteins can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In another embodiment, the Fc domain is derived from IgG4.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In the ACE2 fusion proteins of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

It will be appreciated that the heavy chain constant domains for use in producing an Fc region for the ACE2 fusion proteins of the present disclosure may include variants of the naturally occurring constant domains described above. Such variants may comprise one or more amino acid variations compared to wild type constant domains. In one example, the Fc region of the present disclosure comprises at least one constant domain that varies in sequence from the wild type constant domain. It will be appreciated that the variant constant domains may be longer or shorter than the wild type constant domain.

The Fc domains that are incorporated into the ACE2 fusion proteins of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.4.1.1.

The Fc domains can also be altered to include modifications that improve manufacturability of asymmetric ACE2 fusion proteins, for example by allowing heterodimerization, which is the preferential pairing of non-identical Fc domains over identical Fc domains. Heterodimerization permits the production of ACE2 fusion proteins in which different polypeptide components are connected to one another by an Fc region containing Fc domains that differ in sequence. Examples of heterodimerization strategies are exemplified in Section 6.4.1.2.

It will be appreciated that any of the modifications mentioned above can be combined in any suitable manner to achieve the desired functional properties and/or combined with other modifications to alter the properties of the ACE2 fusion proteins.

Example Fc domain sequences are provided in Table F-1, below, any of which may be incorporated as a component of an ACE2 fusion protein of the present disclosure.

TABLE F-1
Fc Sequences

| | | SEQ |
|---|---|---|
| | | ID |
| Fc | Sequence | NO |
| hlgG1 Fc | EPKSCDKTHTCPPCPAPELLGGPSVFLFPPKPKDTLMISRTPEVTCV | 19 |
| (amino acids 99- | VVDVSHEDPEVKFNWYVDGVEVHNAKTKPREEQYNSTYRVVSVLT | |
| 330 of UniprotKB | VLHQDWLNGKEYKCKVSNKALPAPIEKTISKAKGQPREPQVYTLPPS | |
| P01857-1) | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hlgG2 Fc | ERKCCVECPPCPAPPVAGPSVFLFPPKPKDTLMISRTPEVTCVVVDV | 20 |
| (amino acids 99- | SHEDPEVQFNWYVDGVEVHNAKTKPREEQFNSTFRVVSVLTWVHQ | |
| 326 of UniprotKB | DWLNGKEYKCKVSNKGLPAPIEKTISKTKGQPREPQVYTLPPSREE | |
| P01859-1) | MTKNQVSLTCLVKGFYPSDISVEWESNGQPENNYKTTPPMLDSDGS | |
| | FFLYSKLTVDKSRWQQGNVFSCSVMHEALHNHYTQKSLSLSPGK | |
| hlgG3 Fc | ELKTPLGDTTHTCPRCPEPKSCDTPPPCPRCPEPKSCDTPPPCPRC | 21 |
| (amino acids 99- | PEPKSCDTPPPCPRCPAPELLGGPSVFLFPPKPKDTLMISRTPEVTC | |
| 377 of UniprotKB | VVVDVSHEDPEVQFKWYVDGVEVHNAKTKPREEQYNSTFRVVSVL | |
| P01860-1) | TVLHQDWLNGKEYKCKVSNKALPAPIEKTISKTKGQPREPQVYTLPP | |
| | SREEMTKNQVSLTCLVKGFYPSDIAVEWESSGQPENNYNTTPPMLD | |
| | SDGSFFLYSKLTVDKSRWQQGNIFSCSVMHEALHNRFTQKSLSLSP | |
| | GK | |
| hlgG4 Fc | ESKYGPPCPSCPAPEFLGGPSVFLFPPKPKDTLMISRTPEVTCVVVD | 22 |
| (amino acids 99- | VSQEDPEVQFNWYVDGVEVHNAKTKPREEQFNSTYRVVSVLTVLH | |
| 327 of UniprotKB | QDWLNGKEYKCKVSNKGLPSSIEKTISKAKGQPREPQVYTLPPSQE | |
| P01861-1) | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| hlgG4s Fc | ESKYGPPCPPCPAPPVAGPSVFLFPPKPKDTLMISRTPEVTCVVVDV | 23 |
| | SQEDPEVQFNWYVDGVEVHNAKTKPREEQFNSTYRVVSVLTVLHQ | |
| | DWLNGKEYKCKVSNKGLPSSIEKTISKAKGQPREPQVYTLPPSQEE | |
| | MTKNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| hlgG1 PVA Fc | EPKSCDKTHTCPPCPAPPVAGPSVFLFPPKPKDTLMISRTPEVTCVV | 24 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| hlgG1 PVA star | EPKSCDKTHTCPPCPAPPVAGPSVFLFPPKPKDTLMISRTPEVTCVV | 25 |
| | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLTCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| hlgG1s | DKKVEPKSCDKTHTCPPCPAPPVAGPSVFLFPPKPKDTLMISRTPEV | 26 |
| | TCVVVDVSQEDPEVQFNWYVDGVEVHNAKTKPREEQFNSTYRVVS | |
| | VLTVLHQDWLNGKEYKCKVSNKGLPSSIEKTISKAKGQPREPQVYTL | |
| | PPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSL | |
| | SPGK | |
| hlgG1 N180G, | EPKSCDKTHTCPPCPAPELLGGPSVFLFPPKPKDTLMISRTPEVTCV | 27 |
| also referred | VVDVSHEDPEVKFNWYVDGVEVHNAKTKPREEQYGSTYRVVSVLT | |
| to as N297G | VLHQDWLNGKEYKCKVSNKALPAPIEKTISKAKGQPREPQVYTLPPS | |
| | RDELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hlgG2 variant | ERKCCVECPPCPAPPVAGPSVFLFPPKPKDTLMISRTPEVTCVVVDV | 28 |
| | SHEDPEVQFNWYVDGVEVHNAKTKPREEQFNSTFRVVSVLTVVHQ | |
| | DWLNGKEYKCKVSNKGLPAPIEKTISKTKGQPREPQVYTLPPSREE | |
| | MTKNQVSLTCLVKGFYPSDIAVEWESNGQPENNYKTTPPMLDSDGS | |
| | FFLYSKLTVDKSRWQQGNVFSCSVMHEALHNHYTQKSLSLSPGK | |
| hlgG4 S108P | ESKYGPPCPPCPAPEFLGGPSVFLFPPKPKDTLMISRTPEVTCVVVD | 29 |
| | VSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| lgG1PVA_hinge- | EPKSCDKTHTCPPCPAPPVAGPSVFLFPPKPKDTLMISRTPEVTCVV | 30 |
| Fc knob | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| lgG1PVA_hinge- | EPKSCDKTHTCPPCPAPPVAGPSVFLFPPKPKDTLMISRTPEVTCVV | 31 |
| Fc knob_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| lgG1PVA_hinge- | EPKSCDKTHTCPPCPAPPVAGPSVFLFPPKPKDTLMISRTPEVTCVV | 32 |
| Fc knob_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNHYTQKSLSLSPG | |
| | K | |
| lgG1PVA_hinge- | EPKSCDKTHTCPPCPAPPVAGPSVFLFPPKPKDTLMISRTPEVTCVV | 33 |
| Fc knob_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPCR | |
| | DELTKNQVSLWCLVKGFYPSDIAVEWESNGQPENNYKTTPPVLDSD | |
| | GSFFLYSKLTVDKSRWQQGNVFSCSVMHEALHNRFTQKSLSLSPG | |
| | K | |
| lgG1PVA_hinge- | EPKSCDKTHTCPPCPAPPVAGPSVFLFPPKPKDTLMISRTPEVTCVV | 34 |
| Fc Hole | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| lgG1PVA_hinge- | EPKSCDKTHTCPPCPAPPVAGPSVFLFPPKPKDTLMISRTPEVTCVV | 35 |
| Fc Hole_star | VDVSHEDPEVKFNWYVDGVEVHNAKTKPREEQYNSTYRVVSVLTVL | |
| | HQDWLNGKEYKCKVSNKALPAPIEKTISKAKGQPREPQVYTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |
| lgG1PVA_hinge- | EPKSCDKTHTCPPCPAPPVAGPSVFLFPPKPKDTLMISRTPEVTCVV | 36 |
| Fc Hole_Cys | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNHYTQKSLSLSPG | |
| | K | |
| lgG1PVA_hinge- | EPKSCDKTHTCPPCPAPPVAGPSVFLFPPKPKDTLMISRTPEVTCVV | 37 |
| Fc Hole_Cys_star | VDVSHEDPEVKFNWYVDGVEVHNAKTKPREEQYNSTYRVVSVLTVL | |
| | HQDWLNGKEYKCKVSNKALPAPIEKTISKAKGQPREPQVCTLPPSR | |
| | DELTKNQVSLSCAVKGFYPSDIAVEWESNGQPENNYKTTPPVLDSD | |
| | GSFFLVSKLTVDKSRWQQGNVFSCSVMHEALHNRFTQKSLSLSPG | |
| | K | |

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to any one of the sequences disclosed in Table F-1.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO: 19. In cases where an Fc domain comprises at least 90% sequence identity and less than 100% sequence identity to SEQ ID NO: 19 (e.g., between 90% and 99% sequence identity to SEQ ID NO: 19), an Fc domain may also comprise one or more amino acid substitutions described herein, for example one or more substitutions that reduce effector function (e.g., as described in Section 6.4.1.1) and/or one or more substitutions that promote Fc heterodimerization (e.g., as described in Section 6.4.1.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:20. In cases where an Fc domain comprises at least 90% sequence identity and less than 100% sequence identity to SEQ ID NO:20 (e.g., between 90% and 99% sequence identity to SEQ ID NO:20), an Fc domain may also comprise one or more amino acid substitutions described herein, for example one or more substitutions that reduce effector function (e.g., as described in Section 6.4.1.1) and/or one or more substitutions that promote Fc heterodimerization (e.g., as described in Section 6.4.1.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:21. In cases where an Fc domain comprises at least 90% sequence identity and less than 100% sequence identity to SEQ ID NO:21 (e.g., between 90% and 99% sequence identity to SEQ ID NO:21), an Fc domain may also comprise one or more amino acid substitutions described herein, for example one or more substitutions that reduce effector function (e.g., as described in Section 6.4.1.1) and/or one or more substitutions that promote Fc heterodimerization (e.g., as described in Section 6.4.1.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:22. In cases where an Fc domain comprises at least 90% sequence identity and less than 100% sequence identity to SEQ ID NO:22 (e.g., between 90% and 99% sequence identity to SEQ ID NO:22), an Fc domain may also comprise one or more amino acid substitutions described herein, for example one or more substitutions that reduce effector function (e.g., as described in Section 6.4.1.1) and/or one or more substitutions that promote Fc heterodimerization (e.g., as described in Section 6.4.1.2).

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:23.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:24.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:25.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:26.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:27.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:28.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:29.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:30.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:31.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:32.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:33.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:34.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:35.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:36.

In some aspects, an Fc domain comprises an amino acid sequence having at least about 90%, at least about 91%, at least about 92%, about at least 93%, at least about 94%, at eat least about 95%, at least about 96%, at least about 97%, at least about 98%, at least about 99%, or 100% sequence identity to SEQ ID NO:37.

The ACE2 fusion proteins illustrated in FIGS. 2A-2J typically comprise an Fc homodimer, and thus in some embodiments the Fc domains incorporated into the ACE2 fusion proteins illustrated in FIGS. 2A-2J do not comprise amino acid substitutions that promote Fc heterodimerization.

6.4.1.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an ACE2 fusion polypeptide chain or the Fc region (e.g., one or both Fc domains of an ACE2-Fc fusion construct that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an IgD Fc domain or region, particularly a human IgD Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function. In some embodiments, the IgG1 Fc domain is a variant IgG1 comprising an IgG1 lower hinge domain having the substitution/deletion mutation ELLG→PVA− at amino acid positions 233-236 (EU numbering), sometimes referred to herein as IgG1 PVA. Example IgG1 PVA Fc domain sequences are provided as SEQ ID NOs: 24, 25, and 30-37. An “IgG1 PVA Fc domain” may have only the substitution/deletion mutation ELLG→PVA− at amino acid positions 233-236 (EU numbering), or may have additional mutations such as those described herein. IgG1 PVA Fc domains are also described in PCT Publication No. WO 2023/205753A1, incorporated herein by reference.

In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:24. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:25. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:30. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:31. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:32. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:33. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:34. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:35. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:36. In some embodiments, the IgG1 PVA Fc domain comprises an amino acid sequence having at least 97% sequence identity, at least 98% sequence identity, at least 99% sequence identity, or 100% sequence identity to the amino acid sequence of SEQ ID NO:37.

In another embodiment, the Fc domain is an IgG4 Fc domain with reduced binding to Fc receptors. Exemplary IgG4 Fc domains with reduced binding to Fc receptors may comprise an amino acid sequence selected from Table 2 below. In some embodiments, the Fc domain includes only the bolded portion of the sequences shown below:

TABLE 2

| Fc Domain | Sequence |
|---|---|
| SEQ ID NO: 1 of | Asp Lys Arg Val Glu Ser Lys Tyr Gly Pro Cys Pro Pro Cys Pro |
| WO2014/121087 | Ala Pro Pro Val Ala Gly Pro Ser Val Phe Leu Phe Pro Pro Lys |
| | Pro Lys Asp Thr Leu Met Ile Ser Arg Thr Pro Glu Val Thr Cys |
| | Val Val Val Asp Val Ser Gln Glu Asp Pro Glu Val Gln Phe Asn |
| | Trp Tyr Val Asp Gly Val Glu Val His Asn Ala Lys Thr Lys Pro |
| | Arg Glu Glu Gln Phe Asn Ser Thr Tyr Arg Val Val Ser Val Leu |
| | Thr Val Leu His Gln Asp Trp Leu Asn Gly Lys Glu Tyr Lys Cys |
| | Lys Val Ser Asn Lys Gly Leu Pro Ser Ser Ile Glu Lys Thr Ile |
| | Ser Lys Ala Lys Gly Gln Pro Arg Glu Pro Gln Val Tyr Thr Leu |
| | Pro Pro Ser Gln Glu Glu Met Thr Lys Asn Gln Val Ser Leu Thr |
| | Cys Leu Val Lys Gly Phe Tyr Pro Ser Asp Ile Ala Val Glu Trp |
| | Glu Ser Asn Gly Gln Pro Glu Asn Asn Tyr Lys Thr Thr Pro Pro |
| | Val Leu Asp Ser Asp Gly Ser Phe Phe Leu Tyr Ser Arg Leu Thr |
| | Val Asp Lys Ser Arg Trp Gln Glu Gly Asn Val Phe Ser Cys Ser |
| | Val Met His Glu Ala Leu His Asn His Tyr Thr Gln Lys Ser Leu |
| | Ser Leu Ser Leu Gly Lys (SEQ ID NO: 13) |
| SEQ ID NO: 2 of | Asp Lys Lys Val Glu Pro Lys Ser Cys Asp Lys Thr His Thr Cys |
| WO2014/121087 | Pro Pro Cys Pro Ala Pro Pro Val Ala Gly Pro Ser Val Phe Leu |
| | Phe Pro Pro Lys Pro Lys Asp Thr Leu Met Ile Ser Arg Thr Pro |
| | Glu Val Thr Cys Val Val Val Asp Val Ser Gln Glu Asp Pro Glu |
| | Val Gln Phe Asn Trp Tyr Val Asp Gly Val Glu Val His Asn Ala |
| | Lys Thr Lys Pro Arg Glu Glu Gln Phe Asn Ser Thr Tyr Arg Val |
| | Val Ser Val Leu Thr Val Leu His Gln Asp Trp Leu Asn Gly Lys |
| | Glu Tyr Lys Cys Lys Val Ser Asn Lys Gly Leu Pro Ser Ser Ile |
| | Glu Lys Thr Ile Ser Lys Ala Lys Gly Gln Pro Arg Glu Pro Gln |
| | Val Tyr Thr Leu Pro Pro Ser Arg Asp Glu Leu Thr Lys Asn Gln |
| | Val Ser Leu Thr Cys Leu Val Lys Gly Phe Tyr Pro Ser Asp Ile |
| | Ala Val Glu Trp Glu Ser Asn Gly Gln Pro Glu Asn Asn Tyr Lys |
| | Thr Thr Pro Pro Val Leu Asp Ser Asp Gly Ser Phe Phe Leu Tyr |
| | Ser Lys Leu Thr Val Asp Lys Ser Arg Trp Gln Gln Gly Asn Val |
| | Phe Ser Cys Ser Val Met His Glu Ala Leu His Asn His Tyr Thr |
| | Gln Lys Ser Leu Ser Leu Ser Pro Gly Lys (SEQ ID NO: 18) |
| SEQ ID NO: 30 of | Ala Ser Thr Lys Gly Pro Ser Val Phe Pro Leu Ala Pro Ser Ser |
| WO2014/121087 | Lys |
| | Ser Thr Ser Gly Gly Thr Ala Ala Leu Gly Cys Leu Val Lys Asp |
| | Tyr |
| | Phe Pro Glu Pro Val Thr Val Ser Trp Asn Ser Gly Ala Leu Thr |
| | Ser Gly Val His Thr Phe Pro Ala Val Leu Gln Ser Ser Gly Leu |
| | Tyr Ser Leu Ser Ser Val Val Thr Val Pro Ser Ser Ser Leu Gly |
| | Thr Gln Thr Tyr Ile Cys Asn Val Asn His Lys Pro Ser Asn Thr |
| | Lys Val Asp Lys Lys Val Glu Pro Lys Ser Cys Asp Lys Thr |
| | His Thr Cys Pro Pro Cys Pro Ala Pro Pro Val Ala Gly Pro |
| | Ser Val Phe Leu Phe Pro Pro Lys Pro Lys Asp Thr Leu Met |
| | Ile Ser Arg Thr Pro Glu Val Thr Cys Val Val Val Asp Val Ser |
| | Gln Glu Asp Pro Glu Val Gln Phe Asn Trp Tyr Val Asp Gly |
| | Val Glu Val His Asn Ala Lys Thr Lys Pro Arg Glu Glu Gln |
| | Phe Asn Ser Thr Tyr Arg Val Val Ser Val Leu Thr Val Leu |
| | His Gln Asp Trp Leu Asn Gly Lys Glu Tyr Lys Cys Lys Val |
| | Ser Asn Lys Gly Leu Pro Ser Ser Ile Glu Lys Thr Ile Ser Lys |
| | Ala Lys Gly Gln Pro Arg Glu Pro Gln Val Tyr Thr Leu Pro |
| | Pro Ser Arg Asp Glu Leu Thr Lys Asn Gln Val Ser Leu Thr |
| | Cys Leu Val Lys Gly Phe Tyr Pro Ser Asp Ile Ala Val Glu |
| | Trp Glu Ser Asn Gly Gln Pro Glu Asn Asn |
| | Tyr Lys Thr Thr Pro Pro Val Leu Asp Ser Asp Gly Ser Phe |
| | Phe Leu Tyr Ser Lys Leu Thr Val Asp Lys Ser Arg Trp Gln |
| | Gln Gly Asn Val Phe Ser Cys Ser Val Met His Glu Ala Leu |
| | His Asn His Tyr Thr Gln Lys Ser Leu Ser Leu Ser Pro Gly |
| | Lys (SEQ ID NO: 14) |
| SEQ ID NO: 31 of | Ala Ser Thr Lys Gly Pro Ser Val Phe Pro Leu Ala Pro Cys Ser |
| WO2014/121087 | Arg Ser Thr Ser Glu Ser Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Lys Thr Tyr Thr Cys Asn Val Asp His Lys Pro Ser |
| | Asn Thr Lys Val Asp Lys Arg Val Glu Ser Lys Tyr Gly Pro Pro |
| | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Gln Glu Glu Met Thr Lys Asn |
| | Gln Val Ser Leu Thr Cys Leu Val Lys Gly Phe Tyr Pro Ser |
| | Asp Ile Ala Val Glu Trp Glu Ser Asn Gly Gln Pro Glu Asn |
| | Asn Tyr Lys Thr Thr Pro Pro Val Leu Asp Ser Asp Gly Ser |
| | Phe Phe Leu Tyr Ser Arg Leu Thr Val Asp Lys Ser Arg Trp |
| | Gln Glu Gly Asn Val Phe Ser Cys Ser Val Met His Glu Ala |
| | Leu His Asn His Tyr Thr Gln Lys Ser Leu Ser Leu Ser Leu |
| | Gly Lys (SEQ ID NO: 15) |
| SEQ ID NO: 37 of | Ala Ser Thr Lys Gly Pro Ser Val Phe Pro Leu Ala Pro Ser Ser |
| WO2014/121087 | Lys |
| | Ser Thr Ser Gly Gly Thr Ala Ala Leu Gly Cys Leu Val Lys Asp |
| | Tyr |
| | Phe Pro Glu Pro Val Thr Val Ser Trp Asn Ser Gly Ala Leu Thr |
| | Ser Gly Val His Thr Phe Pro Ala Val Leu Gln Ser Ser Gly Leu |
| | Tyr Ser Leu Ser Ser Val Val Thr Val Pro Ser Ser Ser Leu Gly |
| | Thr Gln Thr Tyr Ile Cys Asn Val Asn His Lys Pro Ser Asn Thr |
| | Lys Val Asp Lys Lys Val Glu Pro Lys Ser Cys Asp Lys Thr |
| | His Thr Cys Pro Pro Cys Pro Ala Pro Pro Val Ala Gly Pro |
| | Ser Val Phe Leu Phe Pro Pro Lys Pro Lys Asp Thr Leu Met |
| | Ile Ser Arg Thr Pro Glu Val Thr Cys Val Val Val Asp Val Ser |
| | Gln Glu Asp Pro Glu Val Gln Phe Asn Trp Tyr Val Asp Gly |
| | Val Glu Val His Asn Ala Lys Thr Lys Pro Arg Glu Glu Gln |
| | Phe Asn Ser Thr Tyr Arg Val Val Ser Val Leu Thr Val Leu |
| | His Gln Asp Trp Leu Asn Gly Lys Glu Tyr Lys Cys Lys Val |
| | Ser Asn Lys Gly Leu Pro Ser Ser Ile Glu Lys Thr Ile Ser Lys |
| | Ala Lys Gly Gln Pro Arg Glu Pro Gln Val Tyr Thr Leu Pro |
| | Pro Ser Arg Asp Glu Leu Thr Lys Asn Gln Val Ser Leu Thr |
| | Cys Leu Val Lys Gly Phe Tyr Pro Ser Asp Ile Ala Val Glu |
| | Trp Glu Ser Asn Gly Gln Pro Glu Asn Asn |
| | Tyr Lys Thr Thr Pro Pro Val Leu Asp Ser Asp Gly Ser Phe |
| | Phe Leu Tyr Ser Lys Leu Thr Val Asp Lys Ser Arg Trp Gln |
| | Gln Gly Asn Val Phe Ser Cys Ser Val Met His Glu Ala Leu |
| | His Asn Arg Phe Thr Gln Lys Ser Leu Ser Leu Ser Pro Gly |
| | Lys (SEQ ID NO: 16) |
| SEQ ID NO: 38 of | Ala Ser Thr Lys Gly Pro Ser Val Phe Pro Leu Ala Pro Cys Ser |
| WO2014/121087 | Arg Ser Thr Ser Glu Ser Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Lys Thr Tyr Thr Cys Asn Val Asp His Lys Pro Ser |
| | Asn Thr Lys Val Asp Lys Arg Val Glu Ser Lys Tyr Gly Pro Pro |
| | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Gln Glu Glu Met Thr Lys Asn |
| | Gln Val Ser Leu Thr Cys Leu Val Lys Gly Phe Tyr Pro Ser |
| | Asp Ile Ala Val Glu Trp Glu Ser Asn Gly Gln Pro Glu Asn |
| | Asn Tyr Lys Thr Thr Pro Pro Val Leu Asp Ser Asp Gly Ser |
| | Phe Phe Leu Tyr Ser Arg Leu Thr Val Asp Lys Ser Arg Trp |
| | Gln Glu Gly Asn Val Phe Ser Cys Ser Val Met His Glu Ala |
| | Leu His Asn Arg Phe Thr Gln Lys Ser Leu Ser Leu Ser Leu |
| | Gly Lys (SEQ ID NO: 17) |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.4.1.2. Fc Heterodimerization Variants

Certain ACE2 fusion proteins entail dimerization between two Fc domains that, unlike a native immunoglobulin, are operably linked to non-identical N-terminal regions, e.g., one Fc domain connected to a Fab and the other Fc domain connected to an ACE2 moiety. Inadequate heterodimerization of two Fc domains to form an Fc region has can be an obstacle for increasing the yield of desired heterodimeric molecules and represents challenges for purification. A variety of approaches available in the art can be used in for enhancing dimerization of Fc domains that might be present in the ACE2 fusion proteins of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides ACE2 fusion proteins comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired ACE2 fusion protein, while homodimerization of identical heavy chains will reduce yield of the desired ACE2 fusion protein. Thus, in a preferred embodiment, the polypeptides that associate to form an ACE2 fusion protein of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL12 receptor agonists comprise a first CH3 domain and a second Ig CH3 domain, wherein the first and second Ig CH3 domains differ from one another by at least one amino acid, and wherein at least one amino acid difference reduces binding of the IL12 receptor agonist to Protein A as compared to a corresponding IL12 receptor agonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.4.2. CH1-CL Domain Pairs

In a naturally occurring immunoglobulin, heavy and light chain pairings are typically stabilized via the natural disulfide bond between the CL domain of the light chain and the CH1 domain of the heavy chain.

The ACE2 fusion proteins of the disclosure may advantageously incorporate a light chain (CL) and a heavy chain constant domain 1 (CH1) to facilitate stable heterodimerization, e.g., between a polypeptide chain comprising an Fc domain and an additional polypeptide chain comprising one or more additional ACE2 moieties.

In some embodiments, the CL is a kappa CL. An exemplary human kappa CL amino acid sequence is provided below (UniProtKB accession number P01834):

IGKC
(SEQ ID NO: 38)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the CL comprises an amino acid sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the amino acid sequence of SEQ ID NO:38.

In other embodiments, the CL is a lambda CL. Exemplary lambda CL amino acid sequences are provided below:

IGLC1 (UniProtKB accession number P0CG04)
(SEQ ID NO: 39)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTECS
IGLC2 (UniProtKB accession number P0DOY2)
(SEQ ID NO: 40)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS
IGLC3 (UniProtKB accession number PODOY3)
(SEQ ID NO: 41)
GQPKAAPSVTLFPPSSEELQANKATLVCLISDFYPGAVTVAWKADSSPVK
AGVETTTPSKQSNNKYAASSYLSLTPEQWKSHKSYSCQVTHEGSTVEKTV
APTECS

In some embodiments, the CL comprises an amino acid sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the amino acid sequence of SEQ ID NO:39. In some embodiments, the CL comprises an amino acid sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the amino acid sequence of SEQ ID NO:40. In some embodiments, the CL comprises an amino acid sequence having at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to the amino acid sequence of SEQ ID NO:41.

Exemplary ACE2 fusion proteins comprising CL-CH1 pairs N-terminal to Fc domains are depicted in FIGS. 2F-2J. In some embodiments, e.g., of the ACE2 fusion protein depicted in FIGS. 2F-2H and 2J, the polypeptide chain comprising the Fc domain comprises a CL domain N-terminal to the Fc domain. In some embodiments, e.g., of the ACE2 fusion protein depicted in FIGS. 2F-2H and 2J, the polypeptide chain comprising the Fc domain comprises a CH1 domain N-terminal to the Fc domain.

In some embodiments, e.g., of the ACE2 fusion protein depicted in FIGS. 2H and 2I, the polypeptide chain comprising the Fc domain comprises a CL domain N-terminal to the Fc domain. In some embodiments, e.g., of the ACE2 fusion protein depicted in FIGS. 2H and 2I, the polypeptide chain comprising the Fc domain comprises a CH1 domain N-terminal to the Fc domain.

### 6.5. Linkers

In certain aspects, the present disclosure provides ACE2 fusion proteins in which two or more components are connected to one another by a peptide linker. By way of example and not limitation, linkers can be used to connect an ACE2 moiety to a multimerization moiety.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker is a G4S linker. In some embodiments the linker comprises two consecutive G4S sequences, three consecutive G4S sequences, four consecutive G4S sequences, five consecutive G4S sequences, or six consecutive G4S sequences.

### 6.5.1. Hinge Sequences

In other embodiments, the ACE2 fusion proteins of the disclosure comprise a linker that is a hinge region. The hinge region can be a native or a modified hinge region. Hinge regions are typically found at the N-termini of Fc regions. The term “hinge region”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric ACE2 fusion proteins formed by the association of two IgG Fc domains) can comprise two associated hinge sequences on separate polypeptide chains.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In some embodiments, an ACE2-Fc fusion construct of the disclosure comprises an Fc region in which one or both Fc domains possesses an intact hinge region at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the ACE2 fusion constructs of the disclosure comprise a modified hinge region that reduces binding affinity for an Fcγ receptor relative to a wild-type hinge region of the same isotype (e.g., human IgG1 or human IgG4).

In one embodiment, the ACE2 fusion constructs of the disclosure comprise an Fc region in which each Fc domain possesses an intact hinge region at its N-terminus, where each Fc domain and hinge region is derived from IgG4 and each hinge region comprise the modified sequence CPPC. The core hinge region of human IgG4 contains the sequence CPSC compared to IgG1 that contains the sequence CPPC. The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.6. Nucleic Acids and Host Cells

In another aspect, the disclosure provides nucleic acids encoding ACE2 fusion proteins of the disclosure. In some embodiments, the ACE2 fusion proteins are encoded by a single nucleic acid. In other embodiments, the ACE2 fusion proteins can be encoded by a plurality (e.g., two, three, four or more) nucleic acids.

A single nucleic acid can encode an ACE2 fusion protein antibody that comprises a single polypeptide chain, an ACE2 fusion protein that comprises two or more polypeptide chains, or a portion of an ACE2 fusion protein that comprises more than two polypeptide chains (for example, a single nucleic acid can encode two polypeptide chains of an ACE2 fusion protein comprising three, four or more polypeptide chains, or three polypeptide chains of an ACE2 fusion protein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, an ACE2 fusion protein comprising two or more polypeptide chains is encoded by two or more nucleic acids. The number of nucleic acids encoding an ACE2 fusion protein can be equal to or less than the number of polypeptide chains in the ACE2 fusion protein (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.6.1. Vectors

The disclosure provides vectors comprising nucleotide sequences encoding an ACE2 fusion protein or a component thereof described herein, for example one or two of the polypeptide chains of an ACE2 fusion protein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.6.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.7. Pharmaceutical Compositions

The ACE2 fusion proteins of the disclosure may be in the form of compositions comprising the ACE2 fusion protein and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the ACE2 fusion proteins and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an ACE2 fusion protein of the disclosure per dose. The quantity of an ACE2 fusion protein included in a unit dose will depend on the disease being treated, as well as other factors as are well known in the art. Such unit dosages may be in the form of a lyophilized dry powder containing an amount of ACE2 fusion protein suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of ACE2 fusion protein suitable for a single administration.

The pharmaceutical compositions may also be supplied in bulk from containing quantities of ACE2 fusion proteins suitable for multiple administrations.

Pharmaceutical compositions may be prepared for storage as lyophilized formulations or aqueous solutions by mixing an ACE2 fusion protein having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of ACE2 fusion protein.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The ACE2 fusion proteins of the disclosure can be formulated as pharmaceutical compositions comprising the ACE2 fusion proteins, for example containing one or more pharmaceutically acceptable excipients or carriers. To prepare pharmaceutical or sterile compositions comprising the ACE2 fusion proteins of the present disclosure, an ACE2 fusion protein preparation can be combined with one or more pharmaceutically acceptable excipient or carrier.

For example, formulations of ACE2 fusion proteins can be prepared by mixing ACE2 fusion proteins with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and WIkins, New York, N. Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

### 6.8. Therapeutic Indications and Methods of Treatment

The present disclosure provides methods for using and applications for the ACE2 fusion proteins of the disclosure.

In certain aspects, the disclosure provides a method of preventing or treating a disease or condition in which an interaction between a RBD of a coronavirus and cellular ACE2 is implicated.

The ACE2 fusion proteins and pharmaceutical compositions of the disclosure can be used to inhibit an interaction between a RBD of a coronavirus and cellular ACE2. In some embodiments, the disclosure provides methods of inhibiting the interaction between the RBD of SARS-CoV. In other embodiments, the disclosure provides methods of inhibiting the interaction between the RBD of SARS-CoV-2. Accordingly, in some embodiments, the disclosure provides methods of inhibiting an interaction between a RBD of a coronavirus and cellular ACE2, comprising administering to a subject in need thereof an ACE2 fusion protein pharmaceutical composition as described herein.

In some embodiments, the disclosure provides methods of administrating an ACE2 fusion protein pharmaceutical composition as described herein to a subject who has been exposed to a coronavirus but is not diagnosed with an infection. In other embodiments, the subject has been tested positive for a coronavirus but is asymptomatic. In yet other embodiments, the subject has been tested positive for a coronavirus and is presymptomatic. In further embodiments, the subject has been tested positive for a coronavirus and is symptomatic. In other embodiments, the subject has developed COVID-19 or another coronavirus-mediated disease or condition.

In some embodiments, the disclosure provides a method of reducing the severity of coronavirus infection, comprising administering to a subject in need thereof the ACE2 fusion protein pharmaceutical composition as described herein.

In some other embodiments, the disclosure provides a method of reducing the viral load of a coronavirus, comprising administering to a subject in need thereof the ACE2 fusion protein pharmaceutical composition as described herein.

In further embodiments, the disclosure provides a method of preventing disease progression in a subject with a coronavirus infection, comprising administering to a subject in need thereof the ACE2 fusion protein pharmaceutical composition as described herein.

In some embodiments, the disclosure provides a method of reducing the duration of a coronavirus infection, comprising administering to a subject in need thereof the ACE2 fusion protein pharmaceutical composition as described herein.

In other embodiments, the disclosure provides a method of reducing the risk of severe disease or death in a subject with a coronavirus infection, comprising administering to a subject in need thereof the ACE2 fusion protein pharmaceutical composition as described herein.

### 6.9. Sequences

Certain sequences of the disclosure are provided in Table S below.

TABLE S
Sequences

| | | SEQ |
|---|---|---|
| | | ID |
| Name | Sequence | NO: |
| human ACE2 | MSSSSWLLLSLVAVTAAQSTIEEQAKTFLDKENHEAEDLFYQSSLASWN | 1 |
| (UniProtKB | YNTNITEENVQNMNNAGDKWSAFLKEQSTLAQMYPLQEIQNLTVKLQLQ | |
| accession | ALQQNGSSVLSEDKSKRLNTILNTMSTIYSTGKVCNPDNPQECLLLEPG | |
| number Q9BYF1) | LNEIMANSLDYNERLWAWESWRSEVGKQLRPLYEEYVVLKNEMARANHY | |
| | EDYGDYWRGDYEVNGVDGYDYSRGQLIEDVEHTFEEIKPLYEHLHAYVR | |
| | AKLMNAYPSYISPIGCLPAHLLGDMWGRFWTNLYSLTVPFGQKPNIDVT | |
| | DAMVDQAWDAQRIFKEAEKFFVSVGLPNMTQGFWENSMLTDPGNVQKAV | |
| | CHPTAWDLGKGDFRILMCTKVTMDDFLTAHHEMGHIQYDMAYAAQPFLL | |
| | RNGANEGFHEAVGEIMSLSAATPKHLKSIGLLSPDFQEDNETEINFLLK | |
| | QALTIVGTLPFTYMLEKWRWMVFKGEIPKDQWMKKWWEMKREIVGVVEP | |
| | VPHDETYCDPASLFHVSNDYSFIRYYTRTLYQFQFQEALCQAAKHEGPL | |
| | HKCDISNSTEAGQKLFNMLRLGKSEPWTLALENVVGAKNMNVRPLLNYF | |
| | EPLFTWLKDQNKNSFVGWSTDWSPYADQSIKVRISLKSALGDKAYEWND | |
| | NEMYLFRSSVAYAMRQYFLKVKNQMILFGEEDVRVANLKPRISENFFVT | |
| | APKNVSDIIPRTEVEKAIRMSRSRINDAFRLNDNSLEFLGIQPTLGPPN | |
| | QPPVSIWLIVFGVVMGVIVVGIVILIFTGIRDRKKKNKARSGENPYASI | |
| | DISKGENNPGFQNTDDVQTSF | |
| ACE2-PD (aa 18- | QSTIEEQAKTFLDKENHEAEDLFYQSSLASWNYNTNITEENVQNMNNAG | 2 |
| 615) | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYAD | |
| ACE2-PD + ND | QSTIEEQAKTFLDKENHEAEDLFYQSSLASWNYNTNITEENVQNMNNAG | 3 |
| (aa 18-740) | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQY | |
| | FLKVKNQMILFGEEDVRVANLKPRISFNFFVTAPKNVSDIIPRTEVEKA | |
| | IRMSRSRINDAFRLNDNSLEFLGIQPTLGPPNQPPVS | |
| SARS-COV RBD | TNLCPFGEVFNATKFPSVYAWERKKISNCVADYSVLYNSTFFSTFKCYG | 4 |
| | VSATKLNDLCFSNVYADSFVVKGDDVRQIAPGQTGVIADYNYKLPDDFM | |
| | GCVLAWNTRNIDATSTGNYNYKYRYLRHGKLRPFERDISNVPFSPDGKP | |
| | CTPPALNCYWPLNDYGFYTTTGIGYQPYRVVVLSFELLNAPATVCGPK | |
| SARS-COV-2 | TNLCPFGEVFNATRFASVYAWNRKRISNCVADYSVLYNSASFSTFKCYG | 5 |
| RBD | VSPTKLNDLCFTNVYADSFVIRGDEVRQIAPGQTGKIADYNYKLPDDFT | |
| | GCVIAWNSNNLDSKVGGNYNYLYRLFRKSNLKPFERDISTEIYQAGSTP | |
| | CNGVEGFNCYFPLQSYGFQPTNGVGYQPYRVVVLSFELLHAPATVCGPK | |
| ACE2 (615)-Fc | QSTIEEQAKTFLDKENHEAEDLFYQSSLASWNYNTNITEENVQNMNNAG | 6 |
| | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADDKTHTCPPCPAPELLGGPSVFLFPPKPKDTLMISRTPEV | |
| | TCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFL | |
| | YSKLTVDKSRWQQGNVFSCSVMHEALHNHYTQKSLSLSPGK | |
| ACE2 (740)-Fc | QSTIEEQAKTFLDKFNHEAEDLFYQSSLASWNYNTNITEENVQNMNNAG | 7 |
| | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQY | |
| | FLKVKNQMILFGEEDVRVANLKPRISFNFFVTAPKNVSDIIPRTEVEKA | |
| | IRMSRSRINDAFRLNDNSLEFLGIQPTLGPPNQPPVSDKTHTCPPCPAP | |
| | ELLGGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSNKALPA | |
| | PIEKTISKAKGQPREPQVYTLPPSRDELTKNQVSLTCLVKGFYPSDIAV | |
| | EWESNGQPENNYKTTPPVLDSDGSFFLYSKLTVDKSRWQQGNVFSCSVM | |
| | HEALHNHYTQKSLSLSPGK | |
| ACE2 v2.4-Fc | QSTIEEQAKYFLDKFNHEAEDLFYQSSLASWNYNTNITEENVQNMNNAG | 8 |
| | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADGGGGSGGGGSGGGGSGGGGSGGGGSGGGGSDKTHTCPPC | |
| | PAPELLGGPSVFLFPPKPKDTLMISRTPEVTCVVVDVSHEDPEVKFNWY | |
| | VDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSD | |
| | IAVEWESNGQPENNYKTTPPVLDSDGSFFLYSKLTVDKSRWQQGNVFSC | |
| | SVMHEALHNHYTQKSLSLSPGK | |
| ACE2 (740/615)- | QSTIEEQAKTFLDKFNHEAEDLFYQSSLASWNYNTNITEENVQNMNNAG | 9 |
| Fc | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQY | |
| | FLKVKNQMILFGEEDVRVANLKPRISFNFFVTAPKNVSDIIPRTEVEKA | |
| | IRMSRSRINDAFRLNDNSLEFLGIQPTLGPPNQPPVSGGGGSGGGGSDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLV | |
| | KGFYPSDIAVEWESNGQPENNYKTTPPVLDSDGSFFLYSKLTVDKSRWQ | |
| | QGNVFSCSVMHEALHNHYTQKSLSLSPGKGGGGSGGGGSGGGGSGGGGS | |
| | QSTIEEQAKTFLDKFNHEAEDLFYQSSLASWNYNTNITEENVQNMNNAG | |
| | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYAD | |
| ACE2 (740/615) | QSTIEEQAKYFLDKFNHEAEDLFYQSSLASWNYNTNITEENVQNMNNAG | 10 |
| v2.4-Fc | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQY | |
| | FLKVKNQMILFGEEDVRVANLKPRISFNFFVTAPKNVSDIIPRTEVEKA | |
| | IRMSRSRINDAFRLNDNSLEFLGIQPTLGPPNQPPVSGGGGSGGGGSDK | |
| | THTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLV | |
| | KGFYPSDIAVEWESNGQPENNYKTTPPVLDSDGSFFLYSKLTVDKSRWQ | |
| | QGNVFSCSVMHEALHNHYTQKSLSLSPGKGGGGSGGGGSGGGGSGGGGS | |
| | QSTIEEQAKYFLDKFNHEAEDLFYQSSLASWNYNTNITEENVQNMNNAG | |
| | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYAD | |
| ACE2-PD v2.4 | QSTIEEQAKYFLDKFNHEAEDLFYQSSLASWNYNTNITEENVQNMNNAG | 11 |
| (aa 18-615) | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYAD | |
| ACE2-PD + ND | QSTIEEQAKYFLDKFNHEAEDLFYQSSLASWNYNTNITEENVQNMNNAG | 12 |
| v2.4 (aa 18-740) | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLFN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLFRSSVAYAMRQY | |
| | FLKVKNQMILFGEEDVRVANLKPRISFNFFVTAPKNVSDIIPRTEVEKA | |
| | IRMSRSRINDAFRLNDNSLEFLGIQPTLGPPNQPPVS | |
| Fc (SEQ ID NO: 1 | DKRVESKYGPPCPPCPAPPVAGPSVFLFPPKPKDTLMISRTPEVTCVVV | 13 |
| of | DVSQEDPEVQFNWYVDGVEVHNAKTKPREEQFNSTYRVVSVLTVLHQDW | |
| WO2014/121087) | LNGKEYKCKVSNKGLPSSIEKTISKAKGQPREPQVYTLPPSQEEMTKNQ | |
| | VSLTCLVKGFYPSDIAVEWESNGQPENNYKTTPPVLDSDGSFFLYSRLT | |
| | VDKSRWQEGNVFSCSVMHEALHNHYTQKSLSLSLGK | |
| Fc (SEQ ID | ASTKGPSVFPLAPSSKSTSGGTAALGCLVKDYFPEPVTVSWNSGALTSG | 14 |
| NO: 30 of | VHTFPAVLQSSGLYSLSSVVTVPSSSLGTQTYICNVNHKPSNTKVDKKV | |
| WO2014/121087) | EPKSCDKTHTCPPCPAPPVAGPSVFLFPPKPKDTLMISRTPEVTCVVVD | |
| | VSQEDPEVQFNWYVDGVEVHNAKTKPREEQFNSTYRVVSVLTVLHQDWL | |
| | NGKEYKCKVSNKGLPSSIEKTISKAKGQPREPQVYTLPPSRDELTKNQV | |
| | SLTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fc (SEQ ID | ASTKGPSVFPLAPCSRSTSESTAALGCLVKDYFPEPVTVSWNSGALTSG | 15 |
| NO: 31 of | VHTFPAVLQSSGLYSLSSVVTVPSSSLGTKTYTCNVDHKPSNTKVDKRV | |
| WO2014/121087) | ESKYGPPCPPCPAPPVAGPSVFLFPPKPKDTLMISRTPEVTCVVVDVSQ | |
| | EDPEVQFNWYVDGVEVHNAKTKPREEQFNSTYRVVSVLTVLHQDWLNGK | |
| | EYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKS | |
| | RWQEGNVFSCSVMHEALHNHYTQKSLSLSLGK | |
| Fc (SEQ ID | ASTKGPSVFPLAPSSKSTSGGTAALGCLVKDYFPEPVTVSWNSGALTSG | 16 |
| NO: 37 of | VHTFPAVLQSSGLYSLSSVVTVPSSSLGTQTYICNVNHKPSNTKVDKKV | |
| WO2014/121087) | EPKSCDKTHTCPPCPAPPVAGPSVFLFPPKPKDTLMISRTPEVTCVVVD | |
| | VSQEDPEVQFNWYVDGVEVHNAKTKPREEQFNSTYRVVSVLTVLHQDWL | |
| | NGKEYKCKVSNKGLPSSIEKTISKAKGQPREPQVYTLPPSRDELTKNQV | |
| | SLTCLVKGFYPSDIAVEWESNGQPENNYKTTPPVLDSDGSFFLYSKLTV | |
| | DKSRWQQGNVFSCSVMHEALHNRFTQKSLSLSPGK | |
| Fc (SEQ ID | ASTKGPSVFPLAPCSRSTSESTAALGCLVKDYFPEPVTVSWNSGALTSG | 17 |
| NO: 38 of | VHTFPAVLQSSGLYSLSSVVTVPSSSLGTKTYTCNVDHKPSNTKVDKRV | |
| WO2014/121087) | ESKYGPPCPPCPAPPVAGPSVFLFPPKPKDTLMISRTPEVTCVVVDVSQ | |
| | EDPEVQFNWYVDGVEVHNAKTKPREEQFNSTYRVVSVLTVLHQDWLNGK | |
| | EYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKS | |
| | RWQEGNVFSCSVMHEALHNRFTQKSLSLSLGK | |
| Fc (SEQ ID NO: 2 | DKKVEPKSCDKTHTCPPCPAPPVAGPSVFLFPPKPKDTLMISRTPEVTC | 18 |
| of | VVVDVSQEDPEVQFNWYVDGVEVHNAKTKPREEQFNSTYRVVSVLTVLH | |
| WO2014/121087) | QDWLNGKEYKCKVSNKGLPSSIEKTISKAKGQPREPQVYTLPPSRDELT | |
| | KNQVSLTCLVKGFYPSDIAVEWESNGQPENNYKTTPPVLDSDGSFFLYS | |
| | KLTVDKSRWQQGNVFSCSVMHEALHNHYTQKSLSLSPGK | |
| hlgG1 Fc | EPKSCDKTHTCPPCPAPELLGGPSVFLFPPKPKDTLMISRTPEVTCVVV | 19 |
| (amino acids 99- | DVSHEDPEVKFNWYVDGVEVHNAKTKPREEQYNSTYRVVSVLTVLHQDW | |
| 330 of UniprotKB | LNGKEYKCKVSNKALPAPIEKTISKAKGQPREPQVYTLPPSRDELTKNQ | |
| P01857-1) | VSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| hlgG2 Fc | ERKCCVECPPCPAPPVAGPSVFLFPPKPKDTLMISRTPEVTCVVVDVSH | 20 |
| (amino acids 99- | EDPEVQFNWYVDGVEVHNAKTKPREEQFNSTFRVVSVLTVVHQDWLNGK | |
| 326 of UniprotKB | EYKCKVSNKGLPAPIEKTISKTKGQPREPQVYTLPPSREEMTKNQVSLT | |
| P01859-1) | CLVKGFYPSDISVEWESNGQPENNYKTTPPMLDSDGSFFLYSKLTVDKS | |
| | RWQQGNVFSCSVMHEALHNHYTQKSLSLSPGK | |
| hlgG3 Fc | ELKTPLGDTTHTCPRCPEPKSCDTPPPCPRCPEPKSCDTPPPCPRCPEP | 21 |
| (amino acids 99- | KSCDTPPPCPRCPAPELLGGPSVFLFPPKPKDTLMISRTPEVTCVVVDV | |
| 377 of UniprotKB | SHEDPEVQFKWYVDGVEVHNAKTKPREEQYNSTFRVVSVLTVLHQDWLN | |
| P01860-1) | GKEYKCKVSNKALPAPIEKTISKTKGQPREPQVYTLPPSREEMTKNQVS | |
| | LTCLVKGFYPSDIAVEWESSGQPENNYNTTPPMLDSDGSFFLYSKLTVD | |
| | KSRWQQGNIFSCSVMHEALHNRFTQKSLSLSPGK | |
| hlgG4 Fc | ESKYGPPCPSCPAPEFLGGPSVFLFPPKPKDTLMISRTPEVTCVVVDVS | 22 |
| (amino acids 99- | QEDPEVQFNWYVDGVEVHNAKTKPREEQFNSTYRVVSVLTVLHQDWLNG | |
| 327 of UniprotKB | KEYKCKVSNKGLPSSIEKTISKAKGQPREPQVYTLPPSQEEMTKNQVSL | |
| P01861-1) | TCLVKGFYPSDIAVEWESNGQPENNYKTTPPVLDSDGSFFLYSRLTVDK | |
| | SRWQEGNVFSCSVMHEALHNHYTQKSLSLSLGK | |
| hlgG4s Fc | ESKYGPPCPPCPAPPVAGPSVFLFPPKPKDTLMISRTPEVTCVVVDVSQ | 23 |
| | EDPEVQFNWYVDGVEVHNAKTKPREEQFNSTYRVVSVLTVLHQDWLNGK | |
| | EYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKS | |
| | RWQEGNVFSCSVMHEALHNHYTQKSLSLSLGK | |
| hlgG1 PVA Fc | EPKSCDKTHTCPPCPAPPVAGPSVFLFPPKPKDTLMISRTPEVTCVVVD | 24 |
| | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLTCLVKGFYPSDIAVEWESNGQPENNYKTTPPVLDSDGSFFLYSKLTV | |
| | DKSRWQQGNVFSCSVMHEALHNHYTQKSLSLSPGK | |
| hlgG1 PVA star | EPKSCDKTHTCPPCPAPPVAGPSVFLFPPKPKDTLMISRTPEVTCVVVD | 25 |
| | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLTCLVKGFYPSDIAVEWESNGQPENNYKTTPPVLDSDGSFFLYSKLTV | |
| | DKSRWQQGNVFSCSVMHEALHNRFTQKSLSLSPGK | |
| hlgG1s | DKKVEPKSCDKTHTCPPCPAPPVAGPSVFLFPPKPKDTLMISRTPEVTC | 26 |
| | VVVDVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSRDELT | |
| | KNQVSLTCLVKGFYPSDIAVEWESNGQPENNYKTTPPVLDSDGSFFLYS | |
| | KLTVDKSRWQQGNVFSCSVMHEALHNHYTQKSLSLSPGK | |
| hlgG1 N180G, | EPKSCDKTHTCPPCPAPELLGGPSVFLFPPKPKDTLMISRTPEVTCVVV | 27 |
| also referred | DVSHEDPEVKFNWYVDGVEVHNAKTKPREEQYGSTYRVVSVLTVLHQDW | |
| to as N297G | LNGKEYKCKVSNKALPAPIEKTISKAKGQPREPQVYTLPPSRDELTKNQ | |
| | VSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| hlgG2 variant | ERKCCVECPPCPAPPVAGPSVFLFPPKPKDTLMISRTPEVTCVVVDVSH | 28 |
| | EDPEVQFNWYVDGVEVHNAKTKPREEQFNSTFRVVSVLTVVHQDWLNGK | |
| | EYKCKVSNKGLPAPIEKTISKTKGQPREPQVYTLPPSREEMTKNQVSLT | |
| | CLVKGFYPSDIAVEWESNGQPENNYKTTPPMLDSDGSFFLYSKLTVDKS | |
| | RWQQGNVFSCSVMHEALHNHYTQKSLSLSPGK | |
| hlgG4 S108P | ESKYGPPCPPCPAPEFLGGPSVFLFPPKPKDTLMISRTPEVTCVVVDVS | 29 |
| | QEDPEVQFNWYVDGVEVHNAKTKPREEQFNSTYRVVSVLTVLHQDWING | |
| | KEYKCKVSNKGLPSSIEKTISKAKGQPREPQVYTLPPSQEEMTKNQVSL | |
| | TCLVKGFYPSDIAVEWESNGQPENNYKTTPPVLDSDGSFFLYSRLTVDK | |
| | SRWQEGNVFSCSVMHEALHNHYTQKSLSLSLGK | |
| lgG1PVA_hinge- | EPKSCDKTHTCPPCPAPPVAGPSVFLFPPKPKDTLMISRTPEVTCVVVD | 30 |
| Fc knob | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLWCLVKGFYPSDIAVEWESNGQPENNYKTTPPVLDSDGSFFLYSKLTV | |
| | DKSRWQQGNVFSCSVMHEALHNHYTQKSLSLSPGK | |
| lgG1PVA_hinge- | EPKSCDKTHTCPPCPAPPVAGPSVELFPPKPKDTLMISRTPEVTCVVVD | 31 |
| Fc knob_star | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLWCLVKGFYPSDIAVEWESNGQPENNYKTTPPVLDSDGSFFLYSKLTV | |
| | DKSRWQQGNVFSCSVMHEALHNRFTQKSLSLSPGK | |
| lgG1PVA_hinge- | EPKSCDKTHTCPPCPAPPVAGPSVFLFPPKPKDTLMISRTPEVTCVVVD | 32 |
| Fc knob_Cys | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPCRDELTKNQV | |
| | SLWCLVKGFYPSDIAVEWESNGQPENNYKTTPPVLDSDGSFFLYSKLTV | |
| | DKSRWQQGNVFSCSVMHEALHNHYTQKSLSLSPGK | |
| lgG1PVA_hinge- | EPKSCDKTHTCPPCPAPPVAGPSVFLFPPKPKDTLMISRTPEVTCVVVD | 33 |
| Fc | VSHEDPEVKFNWYVDGVEVHNAKTKPREEQYNSTYRVVSVLTVLHQDWL | |
| knob_Cys_star | NGKEYKCKVSNKALPAPIEKTISKAKGQPREPQVYTLPPCRDELTKNQV | |
| | SLWCLVKGFYPSDIAVEWESNGQPENNYKTTPPVLDSDGSFFLYSKLTV | |
| | DKSRWQQGNVFSCSVMHEALHNRFTQKSLSLSPGK | |
| lgG1PVA_hinge- | EPKSCDKTHTCPPCPAPPVAGPSVFLFPPKPKDTLMISRTPEVTCVVVD | 34 |
| Fc Hole | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLSCAVKGFYPSDIAVEWESNGQPENNYKTTPPVLDSDGSFFLVSKLTV | |
| | DKSRWQQGNVFSCSVMHEALHNHYTQKSLSLSPGK | |
| lgG1PVA_hinge- | EPKSCDKTHTCPPCPAPPVAGPSVFLFPPKPKDTLMISRTPEVTCVVVD | 35 |
| Fc Hole_star | VSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQV | |
| | SLSCAVKGFYPSDIAVEWESNGQPENNYKTTPPVLDSDGSFFLVSKLTV | |
| | DKSRWQQGNVFSCSVMHEALHNRFTQKSLSLSPGK | |
| lgG1PVA_hinge- | EPKSCDKTHTCPPCPAPPVAGPSVFLFPPKPKDTLMISRTPEVTCVVVD | 36 |
| Fc Hole_Cys | VSHEDPEVKFNWYVDGVEVHNAKTKPREEQYNSTYRVVSVLTVLHQDWL | |
| | NGKEYKCKVSNKALPAPIEKTISKAKGQPREPQVCTLPPSRDELTKNQV | |
| | SLSCAVKGFYPSDIAVEWESNGQPENNYKTTPPVLDSDGSFFLVSKLTV | |
| | DKSRWQQGNVFSCSVMHEALHNHYTQKSLSLSPGK | |
| lgG1PVA_hinge- | EPKSCDKTHTCPPCPAPPVAGPSVFLFPPKPKDTLMISRTPEVTCVVVD | 37 |
| Fc | VSHEDPEVKFNWYVDGVEVHNAKTKPREEQYNSTYRVVSVLTVLHQDWL | |
| Hole_Cys_star | NGKEYKCKVSNKALPAPIEKTISKAKGQPREPQVCTLPPSRDELTKNQV | |
| | SLSCAVKGFYPSDIAVEWESNGQPENNYKTTPPVLDSDGSFFLVSKLTV | |
| | DKSRWQQGNVFSCSVMHEALHNRFTQKSLSLSPGK | |
| Kappa CL (IGKC) | RTVAAPSVFIFPPSDEQLKSGTASVVCLLNNFYPREAKVQWKVDNALQS | 38 |
| | GNSQESVTEQDSKDSTYSLSSTLTLSKADYEKHKVYACEVTHQGLSSPV | |
| | TKSFNRGEC | |
| Lambda CL | GQPKANPTVTLFPPSSEELQANKATLVCLISDFYPGAVTVAWKADGSPV | 39 |
| (IGLC1) | KAGVETTKPSKQSNNKYAASSYLSLTPEQWKSHRSYSCQVTHEGSTVEK | |
| | TVAPTECS | |
| Lambda CL | GQPKAAPSVTLFPPSSEELQANKATLVCLISDFYPGAVTVAWKADSSPV | 40 |
| (IGLC2) | KAGVETTTPSKQSNNKYAASSYLSLTPEQWKSHRSYSCQVTHEGSTVEK | |
| | TVAPTECS | |
| Lambda CL | GQPKAAPSVTLFPPSSEELQANKATLVCLISDFYPGAVTVAWKADSSPV | 41 |
| (IGLC3) | KAGVETTTPSKQSNNKYAASSYLSLTPEQWKSHKSYSCQVTHEGSTVEK | |
| | TVAPTECS | |

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. An ACE2 fusion protein comprising one or more polypeptide chains having the formula:

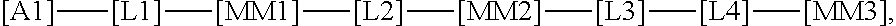

wherein:
(a) [A1] represents an ACE2 moiety;
(b) [L1] represents a linker;
(c) [MM1] represents a multimerization moiety;
(d) [L2] represents a linker;
(e) [MM2] represents a multimerization moiety;
(f) [L3] represents a linker;
(g) [A2] represents an ACE2 moiety;
(h) [L4] represents a linker; and
(i) [MM3] represents a multimerization moiety, and wherein:
   (i) each of [L1], [L2], [L3] and [L4] may be absent;
   (ii) one or two but not all three of [MM1], [MM2] and [MM3] may be absent; and
   (iii) one but not both of [A1] and [A2] may be absent.

2. The ACE2 fusion protein of embodiment 1, wherein [A1] is present and optionally:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of the ACE2 amino acid sequence of SEQ ID NO:1;
(d) comprises at least one amino acid substitution set forth in Table 1;
(e) comprises the amino acid substitutions T27Y, L79T, and N330Y as compared to the ACE2 amino acid sequence of SEQ ID NO:1;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

3. The ACE2 fusion protein of embodiment 2, wherein [A1] comprises an amino acid sequence having at least 90% sequence identity to the PD of ACE2 (SEQ ID NO:2).

4. The ACE2 fusion protein of embodiment 2, wherein [A1] comprises an amino acid sequence having at least 95% sequence identity to the PD of ACE2 (SEQ ID NO:2).

5. The ACE2 fusion protein of embodiment 2, wherein [A1] comprises an amino acid sequence having at least 98% sequence identity to the PD of ACE2 (SEQ ID NO:2).

6. The ACE2 fusion protein of any one of embodiments 1 to 5, wherein [A1] lacks a ND.

7. The ACE2 fusion protein of any one of embodiments 1 to 5, wherein [A1] comprises a ND.

8. The ACE2 fusion protein of embodiment 7, wherein [A1] comprises an amino acid sequence having at least 90% sequence identity to the PD+ND of ACE2 (SEQ ID NO:3).

9. The ACE2 fusion protein of embodiment 7, wherein [A1] comprises an amino acid sequence having at least 95% sequence identity to the PD+ND of ACE2 (SEQ ID NO:3).

10. The ACE2 fusion protein of embodiment 7, wherein [A1] comprises an amino acid sequence having at least 98% sequence identity to the PD+ND of ACE2 (SEQ ID NO:3).

11. The ACE2 fusion protein of any one of embodiments 1 to 10, wherein [A1] comprises at least one amino acid substitution that increases affinity to a coronavirus RBD, e.g., an RBD of SEQ ID NO:4 and/or of SEQ ID NO:5.

12. The ACE2 fusion protein of any one of embodiments 1 to 11, wherein [A1] comprises at least one amino acid substitution at position 25, 27, 31, 34, 42, 79, 90, 92, 324, 325, 330, or 386 of the ACE2 amino acid sequence of SEQ ID NO:1.

13. The ACE2 fusion protein of any one of embodiments 1 to 12, wherein [A1] comprises at least one amino acid substitution set forth in Table 1.

14. The ACE2 fusion protein of any one of embodiments 1 to 13, wherein [A1] comprises the amino acid substitution T27Y as compared to the ACE2 amino acid sequence of SEQ ID NO: 1.

15. The ACE2 fusion protein of any one of embodiments 1 to 14, wherein [A1] comprises the amino acid substitution L79T as compared to the ACE2 amino acid sequence of SEQ ID NO: 1.

16. The ACE2 fusion protein of any one of embodiments 1 to 15, wherein [A1] comprises the amino acid substitution N330Y as compared to the ACE2 amino acid sequence of SEQ ID NO: 1.

17. The ACE2 fusion protein of any one of embodiments 1 to 16, wherein [A1] comprises the amino acid substitutions T27Y, L79T, and N330Y as compared to the ACE2 amino acid sequence of SEQ ID NO:1.

18. The ACE2 fusion protein of any one of embodiments 2 to 17, wherein [A1] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

19. The ACE2 fusion protein of embodiment 18, wherein the increase in affinity is at least 25% as compared to the corresponding sequence in wildtype ACE2 (SEQ ID NO:1).

20 The ACE2 fusion protein of embodiment 18, wherein the increase in affinity is at least 50% as compared to the corresponding sequence in wildtype ACE2 (SEQ ID NO:1).

21. The ACE2 fusion protein of embodiment 18, wherein the increase in affinity is at least 100% as compared to the corresponding sequence in wildtype ACE2 (SEQ ID NO:1).

22. The ACE2 fusion protein of embodiment 18, wherein the increase in affinity is at least 200% as compared to the corresponding sequence in wildtype ACE2 (SEQ ID NO:1).

23. The ACE2 fusion protein of embodiment 18, wherein the increase in affinity is at least 300% as compared to the corresponding sequence in wildtype ACE2 (SEQ ID NO:1).

24. The ACE2 fusion protein of any one of embodiments 1 to 23, in which [L1] is absent.

25. The ACE2 fusion protein of any one of embodiments 1 to 23, in which [L1] is present.

26. The ACE2 fusion protein of embodiment 25, wherein [L1] is 5-35 amino acids in length.

27. The ACE2 fusion protein of embodiment 26, wherein [L1] is 8-15 amino acids in length, e.g., 8, 9, 10, 11, 12, 13, 14 or 15 amino acids.

28. The ACE2 fusion protein of any one of embodiments 1 to 27, in which [MM1] is absent.

29. The ACE2 fusion protein of any one of embodiments 1 to 27, in which [MM1] is present.

30. The ACE2 fusion protein of embodiment 29, wherein [MM1] is a CL domain.

31. The ACE2 fusion protein of embodiment 30, wherein the CL domain is a kappa CL domain.

32. The ACE2 fusion protein of embodiment 30, wherein the CL domain comprises an amino acid sequence having at least 90% sequence identity to the amino acid sequence of SEQ ID NO:38.

33. The ACE2 fusion protein of embodiment 30, wherein the CL domain comprises an amino acid sequence having at least 95% sequence identity to the amino acid sequence of SEQ ID NO:38.

34. The ACE2 fusion protein of embodiment 30, wherein the CL domain comprises an amino acid sequence having at least 98% sequence identity to the amino acid sequence of SEQ ID NO:38.

35. The ACE2 fusion protein of embodiment 30, wherein the CL domain comprises an amino acid sequence having at least 99% sequence identity to the amino acid sequence of SEQ ID NO:38.

36. The ACE2 fusion protein of embodiment 30, wherein the CL domain comprises the amino acid sequence of SEQ ID NO:38.

37. The ACE2 fusion protein of embodiment 30, wherein the CL domain is a lambda CL domain.

38. The ACE2 fusion protein of embodiment 37, wherein the CL domain comprises an amino acid sequence having at least 90% sequence identity to the amino acid sequence of SEQ ID NO:39.

39. The ACE2 fusion protein of embodiment 37, wherein the CL domain comprises an amino acid sequence having at least 95% sequence identity to the amino acid sequence of SEQ ID NO:39.

40. The ACE2 fusion protein of embodiment 37, wherein the CL domain comprises an amino acid sequence having at least 98% sequence identity to the amino acid sequence of SEQ ID NO:39.

41. The ACE2 fusion protein of embodiment 37, wherein the CL domain comprises an amino acid sequence having at least 99% sequence identity to the amino acid sequence of SEQ ID NO:39.

42. The ACE2 fusion protein of embodiment 37, wherein the CL domain comprises the amino acid sequence of SEQ ID NO:39.

43. The ACE2 fusion protein of embodiment 37, wherein the CL domain comprises an amino acid sequence having at least 90% sequence identity to the amino acid sequence of SEQ ID NO:40.

44. The ACE2 fusion protein of embodiment 37, wherein the CL domain comprises an amino acid sequence having at least 95% sequence identity to the amino acid sequence of SEQ ID NO:40.

45. The ACE2 fusion protein of embodiment 37, wherein the CL domain comprises an amino acid sequence having at least 98% sequence identity to the amino acid sequence of SEQ ID NO:40.

46. The ACE2 fusion protein of embodiment 37, wherein the CL domain comprises an amino acid sequence having at least 99% sequence identity to the amino acid sequence of SEQ ID NO:40.

47. The ACE2 fusion protein of embodiment 37, wherein the CL domain comprises the amino acid sequence of SEQ ID NO:40.

48. The ACE2 fusion protein of embodiment 37, wherein the CL domain comprises an amino acid sequence having at least 90% sequence identity to the amino acid sequence of SEQ ID NO:41.

49. The ACE2 fusion protein of embodiment 37, wherein the CL domain comprises an amino acid sequence having at least 95% sequence identity to the amino acid sequence of SEQ ID NO:41.

50. The ACE2 fusion protein of embodiment 37, wherein the CL domain comprises an amino acid sequence having at least 98% sequence identity to the amino acid sequence of SEQ ID NO:41.

51. The ACE2 fusion protein of embodiment 37, wherein the CL domain comprises an amino acid sequence having at least 99% sequence identity to the amino acid sequence of SEQ ID NO:41.

52. The ACE2 fusion protein of embodiment 37, wherein the CL domain comprises the amino acid sequence of SEQ ID NO:41.

53. The ACE2 fusion protein of embodiment 29, wherein [MM1] is a CH1 domain.

54. The ACE2 fusion protein of embodiment 29, wherein [MM1] comprises an Fc domain, [MM2] is a CH1 domain, and [MM3] is absent.

55. The ACE2 fusion protein of embodiment 29, wherein [MM1] comprises an Fc domain, [MM2] is a CL domain, and [MM3] is absent.

56. The ACE2 fusion protein of embodiment 29, wherein [MM1] comprises an Fc domain and [MM2] and [MM3] are absent.

57. The ACE2 fusion protein of any one of embodiments 54 to 56, wherein the Fc domain is an IgG Fc domain.

58. The ACE2 fusion protein of embodiment 57, wherein the Fc domain is an IgG1 or IgG4 Fc domain.

59. The ACE2 fusion protein of embodiment 58, wherein the Fc domain is an IgG4 Fc domain.

60. The ACE2 fusion protein of embodiment 59, wherein the IgG4 Fc domain has reduced binding to an Fc receptor.

61. The ACE2 fusion protein of embodiment 59 or 60, wherein the IgG4 Fc domain comprises a bolded portion of a sequence provided in Table 2.

62. The ACE2 fusion protein of embodiment 59 or 60, wherein the Fc domain is an IgG4s Fc domain.

63. The ACE2 fusion protein of embodiment 58, wherein the Fc domain is an IgG1 PVA Fc.

64. The ACE2 fusion protein of any one of embodiments 54 to 63, wherein the Fc domain comprises a hinge domain.

65. The ACE2 fusion protein of embodiment 64, wherein the hinge domain is a chimeric hinge domain.

66. The ACE2 fusion protein of any one of embodiments 54 to 65, wherein the Fc domain comprises the amino acid substitutions L234A, L235A and P329G (numbered according to Kabat EU index).

67. The ACE2 fusion protein of any one of embodiments 54 to 66, which comprises an Fc dimer.

68. The ACE2 fusion protein of embodiment 67, which comprises an Fc homodimer.

69. The ACE2 fusion protein of any one of embodiments 1 to 68, in which [L2] is absent.

70. The ACE2 fusion protein of any one of embodiments 1 to 68, in which [L2] is present.

71. The ACE2 fusion protein of embodiment 70, wherein [L2] is 5-35 amino acids in length.

72. The ACE2 fusion protein of embodiment 71, wherein [L2] is 15-25 amino acids in length, e.g., 15, 16, 17, 18, 19, 20, 21, 22, 23, 24, or 25 amino acids in length.

73. The ACE2 fusion protein of any one of embodiments 1 to 72, in which [MM2] is absent.

74. The ACE2 fusion protein of any one of embodiments 1 to 72, in which [MM2] is present.

75. The ACE2 fusion protein of embodiment 74, wherein [MM2] comprises an Fc domain.

76. The ACE2 fusion protein of embodiment 75, wherein the Fc domain is an IgG Fc domain.

77. The ACE2 fusion protein of embodiment 76, wherein the Fc domain is an IgG1 or IgG4 Fc domain.

78. The ACE2 fusion protein of embodiment 77, wherein the Fc domain is an IgG4 Fc domain.

79. The ACE2 fusion protein of embodiment 78, wherein the IgG4 Fc domain has reduced binding to an Fc receptor.

80. The ACE2 fusion protein of embodiment 78 or 79, wherein the IgG4 Fc domain comprises a bolded portion of a sequence outlined in Table 2.

81. The ACE2 fusion protein of embodiment 78 or 79, wherein the Fc domain is an IgG4s Fc domain.

82. The ACE2 fusion protein of embodiment 77, wherein the Fc domain is an IgG1 PVA Fc.

83. The ACE2 fusion protein of any one of embodiments 75 to 82, wherein the Fc domain comprises a hinge domain.

84. The ACE2 fusion protein of embodiment 83, wherein the hinge domain is a chimeric hinge domain.

85. The ACE2 fusion protein of any one of embodiments 75 to 84, wherein the Fc domain comprises the amino acid substitutions L234A, L235A and P329G (numbered according to Kabat EU index).

86. The ACE2 fusion protein of any one of embodiments 75 to 85, which comprises an Fc dimer.

87. The ACE2 fusion protein of embodiment 86, which comprises an Fc homodimer.

88. The ACE2 fusion protein of embodiment 74, wherein [MM1] is an Fc domain, [MM2] is a CL domain, and [MM3] is absent.

89. The ACE2 fusion protein of embodiment 88, wherein the CL domain is a kappa CL domain.

90. The ACE2 fusion protein of embodiment 89, wherein the CL domain comprises an amino acid sequence having at least 90% sequence identity to the amino acid sequence of SEQ ID NO:38.

91. The ACE2 fusion protein of embodiment 89, wherein the CL domain comprises an amino acid sequence having at least 95% sequence identity to the amino acid sequence of SEQ ID NO:38.

92. The ACE2 fusion protein of embodiment 89, wherein the CL domain comprises an amino acid sequence having at least 98% sequence identity to the amino acid sequence of SEQ ID NO:38.

93. The ACE2 fusion protein of embodiment 89, wherein the CL domain comprises an amino acid sequence having at least 99% sequence identity to the amino acid sequence of SEQ ID NO:38.

94. The ACE2 fusion protein of embodiment 89, wherein the CL domain comprises the amino acid sequence of SEQ ID NO:38.

95. The ACE2 fusion protein of embodiment 88, wherein the CL domain is a lambda CL domain.

96. The ACE2 fusion protein of embodiment 95, wherein the CL domain comprises an amino acid sequence having at least 90% sequence identity to the amino acid sequence of SEQ ID NO:39.

97. The ACE2 fusion protein of embodiment 95, wherein the CL domain comprises an amino acid sequence having at least 95% sequence identity to the amino acid sequence of SEQ ID NO:39.

98. The ACE2 fusion protein of embodiment 95, wherein the CL domain comprises an amino acid sequence having at least 98% sequence identity to the amino acid sequence of SEQ ID NO:39.

99. The ACE2 fusion protein of embodiment 95, wherein the CL domain comprises an amino acid sequence having at least 99% sequence identity to the amino acid sequence of SEQ ID NO:39.

100. The ACE2 fusion protein of embodiment 95, wherein the CL domain comprises the amino acid sequence of SEQ ID NO:39.

101. The ACE2 fusion protein of embodiment 95, wherein the CL domain comprises an amino acid sequence having at least 90% sequence identity to the amino acid sequence of SEQ ID NO:40.

102. The ACE2 fusion protein of embodiment 95, wherein the CL domain comprises an amino acid sequence having at least 95% sequence identity to the amino acid sequence of SEQ ID NO:40.

103. The ACE2 fusion protein of embodiment 95, wherein the CL domain comprises an amino acid sequence having at least 98% sequence identity to the amino acid sequence of SEQ ID NO:40.

104. The ACE2 fusion protein of embodiment 95, wherein the CL domain comprises an amino acid sequence having at least 99% sequence identity to the amino acid sequence of SEQ ID NO:40.

105. The ACE2 fusion protein of embodiment 95, wherein the CL domain comprises the amino acid sequence of SEQ ID NO:40.

106. The ACE2 fusion protein of embodiment 95, wherein the CL domain comprises an amino acid sequence having at least 90% sequence identity to the amino acid sequence of SEQ ID NO:41.

107. The ACE2 fusion protein of embodiment 95, wherein the CL domain comprises an amino acid sequence having at least 95% sequence identity to the amino acid sequence of SEQ ID NO:41.

108. The ACE2 fusion protein of embodiment 95, wherein the CL domain comprises an amino acid sequence having at least 98% sequence identity to the amino acid sequence of SEQ ID NO:41.

109. The ACE2 fusion protein of embodiment 95, wherein the CL domain comprises an amino acid sequence having at least 99% sequence identity to the amino acid sequence of SEQ ID NO:41.

110. The ACE2 fusion protein of embodiment 95, wherein the CL domain comprises the amino acid sequence of SEQ ID NO:41.

111. The ACE2 fusion protein of embodiment 74, wherein [MM1] is an Fc domain, [MM2] is a CH1 domain, and [MM3] is absent.

112. The ACE2 fusion protein of any one of embodiments 1 to 111, in which [L3] is absent.

113. The ACE2 fusion protein of any one of embodiments 1 to 111, in which [L3] is present.

114. The ACE2 fusion protein of embodiment 113, wherein [L3] is 5-35 amino acids in length.

115. The ACE2 fusion protein of embodiment 114, wherein [L3] is 15-25 amino acids in length, e.g., 15, 16, 17, 18, 19, 20, 21, 22, 23, 24, or 25 amino acids in length.

116. The ACE2 fusion protein of any one of embodiments 1 to 115, wherein [A2] is present and optionally:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of the ACE2 amino acid sequence of SEQ ID NO:1;
(d) comprises at least one amino acid substitution set forth in Table 1;
(e) comprises the amino acid substitutions T27Y, L79T, and N330Y as compared to the ACE2 amino acid sequence of SEQ ID NO:1;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

117. The ACE2 fusion protein of embodiment 116, wherein [A2] comprises an amino acid sequence having at least 90% sequence identity to the PD of ACE2 (SEQ ID NO:2).

118. The ACE2 fusion protein of embodiment 117, wherein [A2] comprises an amino acid sequence having at least 95% sequence identity to the PD of ACE2 (SEQ ID NO:2).

119. The ACE2 fusion protein of embodiment 117, wherein [A2] comprises an amino acid sequence having at least 98% sequence identity to the PD of ACE2 (SEQ ID NO:2).

120. The ACE2 fusion protein of any one of embodiments 65 to 119, wherein [A2] lacks a ND.

121. The ACE2 fusion protein of any one of embodiments 65 to 119, wherein [A2] comprises a ND.

122. The ACE2 fusion protein of embodiment 121, wherein [A2] comprises an amino acid sequence having at least 90% sequence identity to the PD+ND of ACE2 (SEQ ID NO:3).

123. The ACE2 fusion protein of embodiment 121, wherein [A2] comprises an amino acid sequence having at least 95% sequence identity to the PD+ND of ACE2 (SEQ ID NO:3).

124. The ACE2 fusion protein of embodiment 121, wherein [A2] comprises an amino acid sequence having at least 98% sequence identity to the PD+ND of ACE2 (SEQ ID NO:3).

125. The ACE2 fusion protein of any one of embodiments 116 to 124, wherein [A2] comprises at least one amino acid substitution that increases affinity to a coronavirus RBD, e.g., an RBD of SEQ ID NO:4 and/or of SEQ ID NO:5.

126. The ACE2 fusion protein of any one of embodiments 116 to 125, wherein [A2] comprises at least one amino acid substitution at position 25, 27, 31, 34, 42, 79, 90, 92, 324, 325, 330, or 386 of the ACE2 amino acid sequence of SEQ ID NO:1.

127. The ACE2 fusion protein of any one of embodiments 116 to 126, wherein [A2] comprises at least one amino acid substitution set forth in Table 1.

128. The ACE2 fusion protein of any one of embodiments 116 to 127, wherein [A2] comprises the amino acid substitution T27Y as compared to the ACE2 amino acid sequence of SEQ ID NO:1.

129. The ACE2 fusion protein of any one of embodiments 116 to 128, wherein [A21] comprises the amino acid substitution L79T as compared to the ACE2 amino acid sequence of SEQ ID NO: 1.

130. The ACE2 fusion protein of any one of embodiments 116 to 129, wherein [A2] comprises the amino acid substitution N330Y as compared to the ACE2 amino acid sequence of SEQ ID NO: 1.

131. The ACE2 fusion protein of any one of embodiments 116 to 130, wherein [A2] comprises the amino acid substitutions T27Y, L79T, and N330Y as compared to the ACE2 amino acid sequence of SEQ ID NO:1.

132. The ACE2 fusion protein of any one of embodiments 116 to 131, wherein [A2] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

133. The ACE2 fusion protein of embodiment 132, wherein the increase in affinity is at least 25% as compared to the corresponding sequence in wildtype ACE2 (SEQ ID NO:1).

134. The ACE2 fusion protein of embodiment 132, wherein the increase in affinity is at least 50% as compared to the corresponding sequence in wildtype ACE2 (SEQ ID NO:1).

135. The ACE2 fusion protein of embodiment 132, wherein the increase in affinity is at least 100% as compared to the corresponding sequence in wildtype ACE2 (SEQ ID NO:1).

136. The ACE2 fusion protein of embodiment 132, wherein the increase in affinity is at least 200% as compared to the corresponding sequence in wildtype ACE2 (SEQ ID NO:1).

137. The ACE2 fusion protein of embodiment 132, wherein the increase in affinity is at least 300% as compared to the corresponding sequence in wildtype ACE2 (SEQ ID NO:1).

138. The ACE2 fusion protein of any one of embodiments 1 to 137, in which [L4] is absent.

139. The ACE2 fusion protein of any one of embodiments 1 to 137, in which [L4] is present.

140. The ACE2 fusion protein of embodiment 139, wherein [L4] is 5-35 amino acids in length.

141. The ACE2 fusion protein of embodiment 140, wherein [L4] is 15-25 amino acids in length, e.g., 15, 16, 17, 18, 19, 20, 21, 22, 23, 24, or 25 amino acids in length.

142. The ACE2 fusion protein of any one of embodiments 1 to 141, in which [MM3] is absent.

143. The ACE2 fusion protein of any one of embodiments 1 to 141, in which [MM3] is present.

144. The ACE2 fusion protein of embodiment 143, wherein [MM3] is a CL domain.

145. The ACE2 fusion protein of embodiment 144, wherein the CL domain is a kappa CL domain.

146. The ACE2 fusion protein of embodiment 145, wherein the CL domain comprises an amino acid sequence having at least 90% sequence identity to the amino acid sequence of SEQ ID NO:38.

147. The ACE2 fusion protein of embodiment 145, wherein the CL domain comprises an amino acid sequence having at least 95% sequence identity to the amino acid sequence of SEQ ID NO:38.

148. The ACE2 fusion protein of embodiment 145, wherein the CL domain comprises an amino acid sequence having at least 98% sequence identity to the amino acid sequence of SEQ ID NO:38.

149. The ACE2 fusion protein of embodiment 145, wherein the CL domain comprises an amino acid sequence having at least 99% sequence identity to the amino acid sequence of SEQ ID NO:38.

150. The ACE2 fusion protein of embodiment 145, wherein the CL domain comprises the amino acid sequence of SEQ ID NO:38.

151. The ACE2 fusion protein of embodiment 144, wherein the CL domain is a lambda CL domain.

152. The ACE2 fusion protein of embodiment 151, wherein the CL domain comprises an amino acid sequence having at least 90% sequence identity to the amino acid sequence of SEQ ID NO:39.

153. The ACE2 fusion protein of embodiment 151, wherein the CL domain comprises an amino acid sequence having at least 95% sequence identity to the amino acid sequence of SEQ ID NO:39.

154. The ACE2 fusion protein of embodiment 151, wherein the CL domain comprises an amino acid sequence having at least 98% sequence identity to the amino acid sequence of SEQ ID NO:39.

155. The ACE2 fusion protein of embodiment 151, wherein the CL domain comprises an amino acid sequence having at least 99% sequence identity to the amino acid sequence of SEQ ID NO:39.

156. The ACE2 fusion protein of embodiment 151, wherein the CL domain comprises the amino acid sequence of SEQ ID NO:39.

157. The ACE2 fusion protein of embodiment 151, wherein the CL domain comprises an amino acid sequence having at least 90% sequence identity to the amino acid sequence of SEQ ID NO:40.

158. The ACE2 fusion protein of embodiment 151, wherein the CL domain comprises an amino acid sequence having at least 95% sequence identity to the amino acid sequence of SEQ ID NO:40.

159. The ACE2 fusion protein of embodiment 151, wherein the CL domain comprises an amino acid sequence having at least 98% sequence identity to the amino acid sequence of SEQ ID NO:40.

160. The ACE2 fusion protein of embodiment 151, wherein the CL domain comprises an amino acid sequence having at least 99% sequence identity to the amino acid sequence of SEQ ID NO:40.

161. The ACE2 fusion protein of embodiment 151, wherein the CL domain comprises the amino acid sequence of SEQ ID NO:40.

162. The ACE2 fusion protein of embodiment 151, wherein the CL domain comprises an amino acid sequence having at least 90% sequence identity to the amino acid sequence of SEQ ID NO:41.

163. The ACE2 fusion protein of embodiment 151, wherein the CL domain comprises an amino acid sequence having at least 95% sequence identity to the amino acid sequence of SEQ ID NO:41.

164. The ACE2 fusion protein of embodiment 151, wherein the CL domain comprises an amino acid sequence having at least 98% sequence identity to the amino acid sequence of SEQ ID NO:41.

165. The ACE2 fusion protein of embodiment 151, wherein the CL domain comprises an amino acid sequence having at least 99% sequence identity to the amino acid sequence of SEQ ID NO:41.

166. The ACE2 fusion protein of embodiment 151, wherein the CL domain comprises the amino acid sequence of SEQ ID NO:41.

167. The ACE2 fusion protein of embodiment 143, wherein [MM3] is a CH1 domain.

168. An ACE2 fusion protein, which is optionally an ACE2 fusion protein according to any one of embodiments 1 to 167, which comprises one or more polypeptide chains having the formula: [A1]-[L1]-[MM1]-[L2]-[MM2]-[L3]-[A2]-[L4]-[MM3] (“Formula 1”), wherein:
(a) [A1] and [A2] represent ACE2 moieties;
(b) [L1], [L2], [L3], and [L4] represent linkers;
(c) [MM1] represents a multimerization moiety which is a CH1 domain or a CL domain;
(d) [MM2] represents a multimerization moiety which is or comprises an Fc domain; and
(e) [MM3] represents a multimerization moiety which is a CH1 domain or a CL domain.

169. An ACE2 fusion protein, which is optionally an ACE2 fusion protein according to any one of embodiments 1 to 167, which comprises one or more polypeptide chains having the formula: [A1]-[L1]-[MM1] (“Formula 2”), wherein:
(a) [A1] represents an ACE2 moiety;
(b) [L1] represents a linker; and
(c) [MM1] represents a multimerization moiety which comprises an Fc domain.

170. An ACE2 fusion protein, which is optionally an ACE2 fusion protein according to any one of embodiments 1 to 167, which comprises one or more polypeptide chains having the formula: [A1]-[L1]-[MM1]-[L2]-[A2] (“Formula 3”), wherein:
(a) [A1] represents an ACE2 moiety;
(b) [L1] represents a linker;
(c) [MM1] represents a multimerization moiety which comprises an Fc domain;
(d) [L2] represents a linker; and
(e) [A2] represents an ACE2 moiety.

171. An ACE2 fusion protein, which is optionally an ACE2 fusion protein according to any one of embodiments 1 to 167, which comprises one or more polypeptide chains having the formula: [A1]-[L1]-[MM1] (“Formula 4”), wherein:
(a) [A1] represents an ACE2 moiety;
(b) [L1] represents a linker; and
(c) [MM1] represents a multimerization moiety which is a CH1 domain or a CL domain.

172. An ACE2 fusion protein, which is optionally an ACE2 fusion protein according to any one of embodiments 1 to 167, which comprises one or more polypeptide chains having the formula: [A1]-[L1]-[MM1]-[L2]-[MM2] (“Formula 5”), wherein:
(a) [A1] represents an ACE2 moiety;
(b) [L1] represents a linker;
(c) [MM1] represents a multimerization moiety which is a CH1 domain or a CL domain;
(d) [L2] represents a linker; and
(e) [MM2] represents a multimerization moiety which comprises an Fc domain.

173. An ACE2 fusion protein, which is optionally an ACE2 fusion protein according to any one of embodiments 1 to 167, which comprises one or more polypeptide chains having the formula: [A1]-[L1]-[MM1]-[L2]-[MM2]-[L3]-[A2] (“Formula 6”), wherein:
(a) [A1] represents an ACE2 moiety;
(b) [L1] represents a linker;
(c) [MM1] represents a multimerization moiety which is a CH1 domain or a CL domain;
(d) [L2] represents a linker;
(e) [MM2] represents a multimerization moiety which comprises an Fc domain; and
(f) [A2] represents an ACE2 moiety.

174. An ACE2 fusion protein, which is optionally an ACE2 fusion protein according to any one of embodiments 1 to 167, which comprises one or more polypeptide chains having the formula: [MM1]-[L1]-[A1]-[L2]-[MM2] (“Formula 7”), wherein:
(a) [MM1] represents a multimerization moiety which comprises an Fc domain;
(b) [L1] represents a linker;
(c) [A1] represents an ACE2 moiety;
(d) [L2] represents a linker; and
(e) [MM2] represents a multimerization moiety which comprises an Fc domain.

175. An ACE2 fusion protein, which is optionally an ACE2 fusion protein according to any one of embodiments 1 to 167, which comprises one or more polypeptide chains having the formula: [A1]-[L1]-[MM1]-[L2]-[A2] (“Formula 8”), wherein:
(a) [A1] represents an ACE2 moiety;
(b) [L1] represents a linker;
(c) [MM1] represents a multimerization moiety which is a CH1 domain or a CL domain.
(d) [L2] represents a linker; and
(e) [A2] represents an ACE2 moiety.

176. The ACE2 fusion protein of any one of embodiments 169 to 175, wherein each polypeptide chain is associated with an additional polypeptide chain having Formula 4.

177. The ACE2 fusion protein of any one of embodiments 169 to 175, wherein each polypeptide chain is associated with an additional polypeptide chain having Formula 8.

178. An ACE2 fusion protein comprising:
(a) a first polypeptide chain as defined in any one of embodiments 1 to 27, 29, 56-58, and 116-137; and
(b) a second polypeptide chain as defined in according to any one of embodiments 1 to 27, 29, and 56-58,
optionally wherein the ACE2 fusion protein is configured as illustrated in any one of FIGS. 2A-2E.

179. An ACE2 fusion protein comprising:
(a) a first polypeptide chain as defined in any one of embodiments 1 to 27, 29-53, 69-72, 74-87, and 112-137;
(b) a second polypeptide chain as defined in according to any one of embodiments 1 to 27, 29-53, 69-72, 74-87, and 112-137,
(c) a third polypeptide chain as defined in any one of embodiments 1 to 27 or 29-53; and
(d) a fourth polypeptide chain as defined in any one of embodiments 1 to 27 or 29-53,
optionally wherein the ACE2 fusion protein is configured as illustrated in any one of FIGS. 2F, 2G, or 2J.

180. An ACE2 fusion protein comprising:
(a) a first polypeptide chain as defined in any one of embodiments 1-28, 69-72, 75-87, 112-137, and 143-167;
(b) a second polypeptide chain as defined in according to any one of embodiments 1-28, 69-72, 75-87, 112-137, and 143-167,
(c) a third polypeptide chain as defined in any one of embodiments 1 to 27 or 29-53; and
(d) a fourth polypeptide chain as defined in any one of embodiments 1 to 27 or 29-53,
optionally wherein the ACE2 fusion protein is configured as illustrated in FIG. 2I.

181. An ACE2 fusion protein comprising:
(a) a first polypeptide chain as defined in any one of embodiments 1 to 27, 29-53, 69-72, 74-87, 116-137, and 143-167;
(b) a second polypeptide chain as defined in any one of embodiments 1 to 27, 29-53, 69-72, 74-87, 116-137, and 143-167;
(c) a third polypeptide chain as defined in any one of embodiments 1 to 27 or 29-53; and
(d) a fourth polypeptide chain as defined in any one of embodiments 1 to 27 or 29-53;
(e) a fifth polypeptide chain as defined in any one of embodiments 1 to 27 or 29-53; and
(f) a sixth polypeptide chain as defined in any one of embodiments 1 to 27 or 29-53,
optionally wherein the ACE2 fusion protein is configured as illustrated in FIG. 2H.

182. An ACE2 fusion protein, which is optionally an ACE2 fusion protein according to embodiments 1 or embodiment 178, which has the configuration depicted in FIG. 2A.

183. An ACE2 fusion protein, which is optionally an ACE2 fusion protein according to embodiments 1 or embodiment 178, which has the configuration depicted in FIG. 2B.

184. An ACE2 fusion protein, which is optionally an ACE2 fusion protein according to embodiments 1 or embodiment 178, which has the configuration depicted in FIG. 2C.

185. An ACE2 fusion protein, which is optionally an ACE2 fusion protein according to embodiments 1 or embodiment 178, which has the configuration depicted in FIG. 2D.

186. An ACE2 fusion protein, which is optionally an ACE2 fusion protein according to embodiments 1 or embodiment 178, which has the configuration depicted in FIG. 2E.

187. An ACE2 fusion protein, which is optionally an ACE2 fusion protein according to embodiments 1 or embodiment 179, which has the configuration depicted in FIG. 2F.

188. An ACE2 fusion protein, which is optionally an ACE2 fusion protein according to embodiments 1 or embodiment 179, which has the configuration depicted in FIG. 2G.

189. An ACE2 fusion protein, which is optionally an ACE2 fusion protein according to embodiments 1 or embodiment 181, which has the configuration depicted in FIG. 2H.

190. An ACE2 fusion protein, which is optionally an ACE2 fusion protein according to embodiments 1 or embodiment 180, which has the configuration depicted in FIG. 2I.

191. An ACE2 fusion protein, which is optionally an ACE2 fusion protein according to embodiments 1 or embodiment 179, which has the configuration depicted in FIG. 2J.

192. The ACE2 fusion protein of any one of embodiments 1 to 191, which is bivalent for ACE2.

193. The ACE2 fusion protein of any one of embodiments 1 to 191, which is tetravalent for ACE2.

194. The ACE2 fusion protein of any one of embodiments 1 to 191, which is hexavalent for ACE2.

195. The ACE2 fusion protein of any one of embodiments 1 to 191, which is octavalent for ACE2.

196. An ACE2 fusion protein comprising one or more polypeptide chains having the formula:

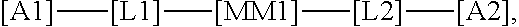

wherein:
(a) [A1] represents a first ACE2 moiety;
(b) [L1] represents a first linker;
(c) [MM1] represent a first multimerization moiety;
(d) [2] represents a second linker; and
(e) [A2] represents a second ACE2 moiety,
wherein [L1] and [L2] are optional and one of [A1] and [A2] is optional.

197. An ACE2 fusion protein, which is optionally an ACE2 fusion protein according to embodiment 196, which has the configuration depicted in FIG. 2A.

198. An ACE2 fusion protein, which is optionally an ACE2 fusion protein according to embodiment 196, which has the configuration depicted in FIG. 2B.

199. An ACE2 fusion protein, which is optionally an ACE2 fusion protein according to embodiment 196, which has the configuration depicted in FIG. 2C.

200. An ACE2 fusion protein, which is optionally an ACE2 fusion protein according to embodiment 196, which has the configuration depicted in FIG. 2D.

201. An ACE2 fusion protein, which is optionally an ACE2 fusion protein according to embodiment 196, which has the configuration depicted in FIG. 2E.

202. The ACE2 fusion protein of any one of embodiments 196 to 201, wherein [A1] is present and optionally:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

203. The ACE2 fusion protein of embodiment 202, wherein [A1] comprises an amino acid sequence having at least 90%, 95% or 98% sequence identity to the PD of ACE2 (SEQ ID NO:2).

204. The ACE2 fusion protein of embodiment 203, wherein [A1] lacks a ND.

205. The ACE2 fusion protein of embodiment 203, wherein [A1] comprises a ND.

206. The ACE2 fusion protein of embodiment 204, wherein [A1] comprises an amino acid sequence having at least 90%, 95% or 98% sequence identity to the PD+ND of ACE2 (SEQ ID NO:3).

207. The ACE2 fusion protein of any one of embodiments 202 to 206, wherein [A1] comprises at least one amino acid substitution that increases affinity to a coronavirus RBD, e.g., an RBD of SEQ ID NO:4 and/or of SEQ ID NO:5.

208. The ACE2 fusion protein of any one of embodiments 202 to 207, wherein [A1] comprises at least one amino acid substitution at position 25, 27, 31, 34, 42, 79, 90, 92, 324, 325, 330, or 386 of ACE2.

209. The ACE2 fusion protein of any one of embodiments 202 to 208, wherein [A1] comprises at least one amino acid substitution set forth in Table 1.

210. The ACE2 fusion protein of any one of embodiments 202 to 209, wherein [A1] comprises the amino acid substitutions T27Y, L79T, and N330Y.

211. The ACE2 fusion protein of any one of embodiments 202 to 210, wherein [A1] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

212. The ACE2 fusion protein of any one of embodiments 196 to 211, wherein [A2] is present and optionally:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

213. The ACE2 fusion protein of embodiment 212, wherein [A2] comprises an amino acid sequence having at least 90%, 95% or 98% sequence identity to the PD of ACE2 (SEQ ID NO:2).

214. The ACE2 fusion protein of embodiment 213, wherein [A2] lacks a ND.

215. The ACE2 fusion protein of embodiment 213, wherein [A2] comprises a ND.

216. The ACE2 fusion protein of embodiment 215, wherein [A2] comprises an amino acid sequence having at least 90%, 95% or 98% sequence identity to the PD+ND of ACE2 (SEQ ID NO:3).

217. The ACE2 fusion protein of any one of embodiments 212 to 216, wherein [A2] comprises at least one amino acid substitution that increases affinity to a coronavirus RBD, e.g., an RBD of SEQ ID NO:4 and/or of SEQ ID NO:5.

218. The ACE2 fusion protein of any one of embodiments 212 to 217, wherein [A2] comprises at least one amino acid substitution at position 25, 27, 31, 34, 42, 79, 90, 92, 324, 325, 330, or 386 of ACE2.

219. The ACE2 fusion protein of any one of embodiments 212 to 218, wherein [A2] comprises at least one amino acid substitution set forth in Table 1.

220. The ACE2 fusion protein of any one of embodiments 212 to 219, wherein [A2] comprises the amino acid substitutions T27Y, L79T, and N330Y.

221. The ACE2 fusion protein of any one of embodiments 212 to 220, wherein [A2] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

222. The ACE2 fusion protein of any one of embodiments 196 to 221, in which [L1] is absent.

223. The ACE2 fusion protein of any one of embodiments 196 to 221, in which if [A1] is present, [L1] is also present.

224. The ACE2 fusion protein of embodiment 223, wherein [L1] is 5-35 amino acids in length.

225. The ACE2 fusion protein of embodiment 224, wherein [L1] is 8-15 amino acids in length, e.g., 8, 9, 10, 11, 12, 13, 14 or 15 amino acids.

226. The ACE2 fusion protein of any one of embodiments 196 to 225, in which [L2] is absent.

227. The ACE2 fusion protein of any one of embodiments 196 to 225, in which if [A2] is present, [L2] is also present.

228. The ACE2 fusion protein of embodiment 227, wherein [L2] is 5-35 amino acids in length.

229. The ACE2 fusion protein of embodiment 228, wherein [L2] is 15-25 amino acids in length, e.g., 15, 16, 17, 18, 19, 20, 21, 22, 23, 24, or 25 amino acids in length.

230. The ACE2 fusion protein of any one of embodiments 196 to 229, which is bivalent for ACE2.

231. The ACE2 fusion protein of any one of embodiments 196 to 229, which is tetravalent for ACE2.

232. An ACE2 fusion protein according to any one of embodiments 196 to 229, wherein the one or more polypeptide chains has the formula:

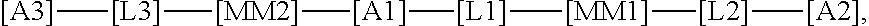

wherein
(a) [A3] represents a third ACE2 moiety;
(b) [L3] represents a third linker; and
(c) [MM2] represents a second multimerization moiety,
wherein [L3] is optional.

233. The ACE2 fusion protein according to embodiment 232, wherein each polypeptide chain having the formula [A3]-[L3]-[MM2]-[A1]-[L1]-[MM1]-[L2]-[A2] is associated with a polypeptide chain having the formula [A4]-[L4]-[MM3]-[L5]-[A5], wherein:
(a) [A4] represents a fourth ACE2 moiety,
(b) [L4] represents a fourth linker,
(c) [MM3] represents a third multimerization moiety that is capable of associating with [MM2],
(d) [L5] is a fifth linker,
(e) [A5] is a fifth ACE2 moiety,
wherein [L4], [L5] and [A5] are optional.

234. An ACE2 fusion protein, which is optionally an ACE2 fusion protein according to embodiment 232 or embodiment 233, which has the configuration depicted in FIG. 2F.

235. An ACE2 fusion protein, which is optionally an ACE2 fusion protein according to embodiment 232 or embodiment 233, which has the configuration depicted in FIG. 2G.

236. The ACE2 fusion protein of any one of embodiments 232 to 235, wherein [A3]:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

237. The ACE2 fusion protein of embodiment 236, wherein [A3] comprises an amino acid sequence having at least 90%, 95% or 98% sequence identity to the PD of ACE2 (SEQ ID NO:2).

238. The ACE2 fusion protein of embodiment 237, wherein [A3] lacks a ND.

239. The ACE2 fusion protein of embodiment 237, wherein [A3] comprises a ND.

240. The ACE2 fusion protein of embodiment 239, wherein [A3] comprises an amino acid sequence having at least 90%, 95% or 98% sequence identity to the PD+ND of ACE2 (SEQ ID NO:3).

241. The ACE2 fusion protein of any one of embodiments 236 to 240, wherein [A3] comprises at least one amino acid substitution that increases affinity to a coronavirus RBD, e.g., an RBD of SEQ ID NO:4 and/or of SEQ ID NO:5.

242. The ACE2 fusion protein of any one of embodiments 236 to 241, wherein [A3] comprises at least one amino acid substitution at position 25, 27, 31, 34, 42, 79, 90, 92, 324, 325, 330, or 386 of ACE2.

243. The ACE2 fusion protein of any one of embodiments 236 to 242, wherein [A3] comprises at least one amino acid substitution set forth in Table 1.

244. The ACE2 fusion protein of any one of embodiments 236 to 243, wherein [A3] comprises the amino acid substitutions T27Y, L79T, and N330Y.

245. The ACE2 fusion protein of any one of embodiments 236 to 244, wherein [A3] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

246. The ACE2 fusion protein of any one of embodiments 232 to 245, in which [L3] is present.

247. The ACE2 fusion protein of embodiment 246, wherein [L3] is 5-35 amino acids in length.

248. The ACE2 fusion protein of embodiment 247, wherein [L3] is 8-15 amino acids in length, e.g., 8, 9, 10, 11, 12, 13, 14 or 15 amino acids.

249. The ACE2 fusion protein of any one of embodiments 232 to 248, wherein [MM2] is a CL domain.

250. The ACE2 fusion protein of any one of embodiments 232 to 248, wherein [MM2] is a CH1 domain.

251. The ACE2 fusion protein of any one of embodiments 233 to 250, wherein [A4]:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

252. The ACE2 fusion protein of embodiment 251, wherein [A4] comprises an amino acid sequence having at least 90%, 95% or 98% sequence identity to the PD of ACE2 (SEQ ID NO:2).

253. The ACE2 fusion protein of embodiment 252, wherein [A4] lacks a ND.

254. The ACE2 fusion protein of embodiment 252, wherein [A4] comprises a ND.

255. The ACE2 fusion protein of embodiment 254, wherein [A4] comprises an amino acid sequence having at least 90%, 95% or 98% sequence identity to the PD+ND of ACE2 (SEQ ID NO:3).

256. The ACE2 fusion protein of any one of embodiments 251 to 255, wherein [A4] comprises at least one amino acid substitution that increases affinity to a coronavirus RBD, e.g., an RBD of SEQ ID NO:4 and/or of SEQ ID NO:5.

257. The ACE2 fusion protein of any one of embodiments 251 to 256, wherein [A4] comprises at least one amino acid substitution at position 25, 27, 31, 34, 42, 79, 90, 92, 324, 325, 330, or 386 of ACE2.

258. The ACE2 fusion protein of any one of embodiments 251 to 257, wherein [A4] comprises at least one amino acid substitution set forth in Table 1.

259. The ACE2 fusion protein of any one of embodiments 251 to 258, wherein [A4] comprises the amino acid substitutions T27Y, L79T, and N330Y.

260. The ACE2 fusion protein of any one of embodiments 251 to 259, wherein [A4] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

261. The ACE2 fusion protein of any one of embodiments 233 to 260, in which [L4] is present.

262. The ACE2 fusion protein of embodiment 261, wherein [L4] is 5-35 amino acids in length.

263. The ACE2 fusion protein of embodiment 262, wherein [L4] is 8-15 amino acids in length, e.g., 8, 9, 10, 11, 12, 13, 14 or 15 amino acids.

264. The ACE2 fusion protein of any one of embodiments 233 to 263, wherein [MM3] is a CL domain and [MM2] is a CH1 domain.

265. The ACE2 fusion protein of any one of embodiments 233 to 263, wherein [MM3] is a CH1 domain and [MM2] is a CL domain.

266. The ACE2 fusion protein of any one of embodiments 233 to 265, in which [L5] is absent.

267. The ACE2 fusion protein of any one of embodiments 233 to 265, in which if [A5] is present, [L5] is also present.

268. The ACE2 fusion protein of embodiment 267, wherein [L5] is 5-35 amino acids in length.

269. The ACE2 fusion protein of embodiment 268, wherein [L5] is 8-15 amino acids in length, e.g., 8, 9, 10, 11, 12, 13, 14 or 15 amino acids.

270. The ACE2 fusion protein of any one of embodiments 233 to 269, wherein [A5] is present and optionally:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

271. The ACE2 fusion protein of embodiment 270, wherein [A5] comprises an amino acid sequence having at least 90%, 95% or 98% sequence identity to the PD of ACE2 (SEQ ID NO:2).

272. The ACE2 fusion protein of embodiment 271, wherein [A5] lacks a ND.

273. The ACE2 fusion protein of embodiment 271, wherein [A5] comprises a ND.

274. The ACE2 fusion protein of embodiment 273, wherein [A5] comprises an amino acid sequence having at least 90%, 95% or 98% sequence identity to the PD+ND of ACE2 (SEQ ID NO:3).

275. The ACE2 fusion protein of any one of embodiments 270 to 274, wherein [A5] comprises at least one amino acid substitution that increases affinity to a coronavirus RBD, e.g., an RBD of SEQ ID NO:4 and/or of SEQ ID NO:5.

276. The ACE2 fusion protein of any one of embodiments 270 to 275, wherein [A5] comprises at least one amino acid substitution at position 25, 27, 31, 34, 42, 79, 90, 92, 324, 325, 330, or 386 of ACE2.

277. The ACE2 fusion protein of any one of embodiments 270 to 276, wherein [A5] comprises at least one amino acid substitution set forth in Table 1.

278. The ACE2 fusion protein of any one of embodiments 270 to 277, wherein [A5] comprises the amino acid substitutions T27Y, L79T, and N330Y.

279. The ACE2 fusion protein of any one of embodiments 270 to 278, wherein [A5] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

280. The ACE2 fusion protein of any one of embodiments 232 to 279, which is hexavalent for ACE2.

281. The ACE2 fusion protein of any one of embodiments 232 to 279, which is octavalent for ACE2.

282. The ACE2 fusion protein of any one of embodiments 196 to 281, wherein [MM1] comprises an Fc domain.

283. The ACE2 fusion protein of embodiment 282, wherein the Fc domain is an IgG Fc domain.

284. The ACE2 fusion protein of embodiment 283, wherein the Fc domain is an IgG1 or IgG4 Fc domain.

285. The ACE2 fusion protein of embodiment 284, wherein the Fc domain comprises a hinge domain.

286. The ACE2 fusion protein of embodiment 285, wherein the hinge domain is a chimeric hinge domain.

287. The ACE2 fusion protein of any one of embodiments 196 to 286, which comprises an Fc dimer.

288. The ACE2 fusion protein of embodiment 287, which comprises an Fc homodimer.

289. A nucleic acid or plurality of nucleic acids encoding the ACE2 fusion protein of any one of embodiments 1 to 288.

290. A host cell engineered to express the ACE2 fusion protein of any one of embodiments 1 to 288 or the nucleic acid(s) of embodiment 289.

291. A method of producing the ACE2 fusion protein of any one of embodiments 1 to 288, comprising culturing the host cell of embodiment 290 and recovering the ACE2 fusion protein expressed thereby.

292. A pharmaceutical composition comprising the ACE2 fusion protein of any one of embodiments 1 to 288 and an excipient.

293. A method of treating a coronavirus disease, comprising administering to a subject in need thereof the ACE2 fusion protein of any one of 1 to 288 or the pharmaceutical composition of embodiment 292.

294. A method of inhibiting an interaction between a RBD of a coronavirus and cellular ACE2, comprising administering to a subject in need thereof the ACE2 fusion protein of any one of embodiments 1 to 288 or the pharmaceutical composition of embodiment 292.

295. A method reducing the severity of coronavirus infection, comprising administering to a subject in need thereof the ACE2 fusion protein of any one of embodiments 1 to 288 or the pharmaceutical composition of embodiment 292.

296. A method of reducing the viral load of a coronavirus, comprising administering to a subject in need thereof the ACE2 fusion protein of any one of embodiments 1 to 288 or the pharmaceutical composition of embodiment 292.

297. A method of preventing disease progression in a subject with a coronavirus infection, comprising administering to a subject in need thereof the ACE2 fusion protein of any one of embodiments 1 to 288 or the pharmaceutical composition of embodiment 292.

298. A method of reducing the duration of a coronavirus infection, comprising administering to a subject in need thereof the ACE2 fusion protein of any one of embodiments 1 to 288 or the pharmaceutical composition of embodiment 292.

299. A method of reducing the risk of severe disease or death in a subject with a coronavirus infection, comprising administering to a subject in need thereof the ACE2 fusion protein of any one of embodiments 1 to 288 or the pharmaceutical composition of embodiment 292.

300. The method of any one of embodiments 293 to 299, wherein the coronavirus is SARS-CoV.

301. The method of any one of embodiments 289 to 295, wherein the coronavirus is SARS-CoV-2.

### 8. EXAMPLES

### 8.1. Materials and Methods

### 8.1.1. Design, Construction, and Production of ACE2 Fusion Proteins

ACE2 fusion proteins were designed as DNA fragments and cloned into the mammalian expression vector pcDNA3.4. For example, an exemplary tetravalent ACE2 fusion protein was designed as a DNA fragment with the following components from 5′ to 3′ end: an mROR1 signal sequence, an ACE2 ectodomain (18-740 amino acids from the N-terminal, ACE2 (740)), 2xG₄S linker (L1), a human IgG1 Fc domain, 4xG₄S linker (L2), and a second ACE2 ectodomain (18-615 aa, ACE2 (615)). Next, the DNA fragment was synthesized and cloned into the mammalian expression vector pcDNA3.4. Using the reported v2.4 mutations (T27Y, L79T, N330Y), an affinity matured version of tetravalent ACE2-Fc (ACE2 v2.4 (740/615)-Fc (IgG)) was created for comparison.

Sequences and/or descriptions for exemplary constructs are presented below in Table 3.

TABLE 3

| | | SEQ |
|---|---|---|
| | | ID |
| Construct | Sequence and/or Description | NO |
| ACE2 (615)-Fc | QSTIEEQAKTFLDKFNHEAEDLFYQSSLASWNYNTNITEENVQNMNNAGDKW | 6 |
| | SAFLKEQSTLAQMYPLQEIQNLTVKLQLQALQQNGSSVLSEDKSKRLNTILNT | |
| | MSTIYSTGKVCNPDNPQECLLLEPGLNEIMANSLDYNERLWAWESWRSEVG | |
| | KQLRPLYEEYVVLKNEMARANHYEDYGDYWRGDYEVNGVDGYDYSRGQLIE | |
| | DVEHTFEEIKPLYEHLHAYVRAKLMNAYPSYISPIGCLPAHLLGDMWGRFWTN | |
| | LYSLTVPFGQKPNIDVTDAMVDQAWDAQRIFKEAEKFFVSVGLPNMTQGFWE | |
| | NSMLTDPGNVQKAVCHPTAWDLGKGDFRILMCTKVTMDDFLTAHHEMGHIQ | |
| | YDMAYAAQPFLLRNGANEGFHEAVGEIMSLSAATPKHLKSIGLLSPDFQEDNE | |
| | TEINFLLKQALTIVGTLPFTYMLEKWRWMVFKGEIPKDQWMKKWWEMKREIV | |
| | GVVEPVPHDETYCDPASLFHVSNDYSFIRYYTRTLYQFQFQEALCQAAKHEG | |
| | PLHKCDISNSTEAGQKLFNMLRLGKSEPWTLALENVVGAKNMNVRPLLNYFE | |
| | PLFTWLKDQNKNSFVGWSTDWSPYADDKTHTCPPCPAPELLGGPSVFLFPP | |
| | KPKDTLMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPREPQVYT | |
| | LPPSRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| ACE2 (740)-Fc | QSTIEEQAKTFLDKFNHEAEDLFYQSSLASWNYNTNITEENVQNMNNAGDKW | 7 |
| | SAFLKEQSTLAQMYPLQEIQNLTVKLQLQALQQNGSSVLSEDKSKRLNTILNT | |
| | MSTIYSTGKVCNPDNPQECLLLEPGLNEIMANSLDYNERLWAWESWRSEVG | |
| | KQLRPLYEEYVVLKNEMARANHYEDYGDYWRGDYEVNGVDGYDYSRGQLIE | |
| | DVEHTFEEIKPLYEHLHAYVRAKLMNAYPSYISPIGCLPAHLLGDMWGRFWTN | |
| | LYSLTVPFGQKPNIDVTDAMVDQAWDAQRIFKEAEKFFVSVGLPNMTQGFWE | |
| | NSMLTDPGNVQKAVCHPTAWDLGKGDFRILMCTKVTMDDFLTAHHEMGHIQ | |
| | YDMAYAAQPFLLRNGANEGFHEAVGEIMSLSAATPKHLKSIGLLSPDFQEDNE | |
| | TEINFLLKQALTIVGTLPFTYMLEKWRWMVFKGEIPKDQWMKKWWEMKREIV | |
| | GVVEPVPHDETYCDPASLFHVSNDYSFIRYYTRTLYQFQFQEALCQAAKHEG | |
| | PLHKCDISNSTEAGQKLFNMLRLGKSEPWTLALENVVGAKNMNVRPLLNYFE | |
| | PLFTWLKDQNKNSFVGWSTDWSPYADQSIKVRISLKSALGDKAYEWNDNEM | |
| | YLFRSSVAYAMRQYFLKVKNQMILFGEEDVRVANLKPRISFNFFVTAPKNVSDI | |
| | IPRTEVEKAIRMSRSRINDAFRLNDNSLEFLGIQPTLGPPNQPPVSDKTHTCPP | |
| | CPAPELLGGPSVFLFPPKPKDTLMISRTPEVTCVVVDVSHEDPEVKFNWYVD | |
| | GVEVHNAKTKPREEQYNSTYRVVSVLTVLHQDWLNGKEYKCKVSNKALPAPI | |
| | EKTISKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALHNHY | |
| | TQKSLSLSPGK | |
| ACE2 v2.4-Fc | QSTIEEQAKYFLDKFNHEAEDLFYQSSLASWNYNTNITEENVQNMNNAGDKW | 8 |
| | SAFLKEQSTTAQMYPLQEIQNLTVKLQLQALQQNGSSVLSEDKSKRLNTILNT | |
| | MSTIYSTGKVCNPDNPQECLLLEPGLNEIMANSLDYNERLWAWESWRSEVG | |
| | KQLRPLYEEYVVLKNEMARANHYEDYGDYWRGDYEVNGVDGYDYSRGQLIE | |
| | DVEHTFEEIKPLYEHLHAYVRAKLMNAYPSYISPIGCLPAHLLGDMWGRFWTN | |
| | LYSLTVPFGQKPNIDVTDAMVDQAWDAQRIFKEAEKFFVSVGLPNMTQGFWE | |
| | YSMLTDPGNVQKAVCHPTAWDLGKGDFRILMCTKVTMDDFLTAHHEMGHIQ | |
| | YDMAYAAQPFLLRNGANEGFHEAVGEIMSLSAATPKHLKSIGLLSPDFQEDNE | |
| | TEINFLLKQALTIVGTLPFTYMLEKWRWMVFKGEIPKDQWMKKWWEMKREIV | |
| | GVVEPVPHDETYCDPASLFHVSNDYSFIRYYTRTLYQFQFQEALCQAAKHEG | |
| | PLHKCDISNSTEAGQKLFNMLRLGKSEPWTLALENVVGAKNMNVRPLLNYFE | |
| | PLFTWLKDQNKNSFVGWSTDWSPYADGGGGSGGGGSGGGGSGGGGSGG | |
| | GGSGGGGSDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRWQQ | |
| | GNVFSCSVMHEALHNHYTQKSLSLSPGK | |
| ACE2 (740/615)- | QSTIEEQAKTFLDKFNHEAEDLFYQSSLASWNYNTNITEENVQNMNNAGDKW | 9 |
| Fc | SAFLKEQSTLAQMYPLQEIQNLTVKLQLQALQQNGSSVLSEDKSKRLNTILNT | |
| | MSTIYSTGKVCNPDNPQECLLLEPGLNEIMANSLDYNERLWAWESWRSEVG | |
| | KQLRPLYEEYVVLKNEMARANHYEDYGDYWRGDYEVNGVDGYDYSRGQLIE | |
| | DVEHTFEEIKPLYEHLHAYVRAKLMNAYPSYISPIGCLPAHLLGDMWGRFWTN | |
| | LYSLTVPFGQKPNIDVTDAMVDQAWDAQRIFKEAEKFFVSVGLPNMTQGFWE | |
| | NSMLTDPGNVQKAVCHPTAWDLGKGDFRILMCTKVTMDDFLTAHHEMGHIQ | |
| | YDMAYAAQPFLLRNGANEGFHEAVGEIMSLSAATPKHLKSIGLLSPDFQEDNE | |
| | TEINFLLKQALTIVGTLPFTYMLEKWRWMVFKGEIPKDQWMKKWWEMKREIV | |
| | GVVEPVPHDETYCDPASLFHVSNDYSFIRYYTRTLYQFQFQEALCQAAKHEG | |
| | PLHKCDISNSTEAGQKLFNMLRLGKSEPWTLALENVVGAKNMNVRPLLNYFE | |
| | PLFTWLKDQNKNSFVGWSTDWSPYADQSIKVRISLKSALGDKAYEWNDNEM | |
| | YLFRSSVAYAMRQYFLKVKNQMILFGEEDVRVANLKPRISFNFFVTAPKNVSDI | |
| | IPRTEVEKAIRMSRSRINDAFRLNDNSLEFLGIQPTLGPPNQPPVSGGGGSGG | |
| | GGSDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVKGFYPS | |
| | DIAVEWESNGQPENNYKTTPPVLDSDGSFFLYSKLTVDKSRWQQGNVFSCS | |
| | VMHEALHNHYTQKSLSLSPGKGGGGSGGGGSGGGGSGGGGSQSTIEEQAK | |
| | TFLDKFNHEAEDLFYQSSLASWNYNTNITEENVQNMNNAGDKWSAFLKEQST | |
| | LAQMYPLQEIQNLTVKLQLQALQQNGSSVLSEDKSKRLNTILNTMSTIYSTGKV | |
| | CNPDNPQECLLLEPGLNEIMANSLDYNERLWAWESWRSEVGKQLRPLYEEY | |
| | VVLKNEMARANHYEDYGDYWRGDYEVNGVDGYDYSRGQLIEDVEHTFEEIK | |
| | PLYEHLHAYVRAKLMNAYPSYISPIGCLPAHLLGDMWGRFWTNLYSLTVPFG | |
| | QKPNIDVTDAMVDQAWDAQRIFKEAEKFFVSVGLPNMTQGFWENSMLTDPG | |
| | NVQKAVCHPTAWDLGKGDFRILMCTKVTMDDFLTAHHEMGHIQYDMAYAAQ | |
| | PFLLRNGANEGFHEAVGEIMSLSAATPKHLKSIGLLSPDFQEDNETEINFLLKQ | |
| | ALTIVGTLPFTYMLEKWRWMVFKGEIPKDQWMKKWWEMKREIVGVVEPVPH | |
| | DETYCDPASLFHVSNDYSFIRYYTRTLYQFQFQEALCQAAKHEGPLHKCDISN | |
| | STEAGQKLFNMLRLGKSEPWTLALENVVGAKNMNVRPLLNYFEPLFTWLKDQ | |
| | NKNSFVGWSTDWSPYAD | |
| ACE2 (740/615) | QSTIEEQAKYFLDKFNHEAEDLFYQSSLASWNYNTNITEENVQNMNNAGDKW | 10 |
| v2.4-Fc | SAFLKEQSTTAQMYPLQEIQNLTVKLQLQALQQNGSSVLSEDKSKRLNTILNT | |
| | MSTIYSTGKVCNPDNPQECLLLEPGLNEIMANSLDYNERLWAWESWRSEVG | |
| | KQLRPLYEEYVVLKNEMARANHYEDYGDYWRGDYEVNGVDGYDYSRGQLIE | |
| | DVEHTFEEIKPLYEHLHAYVRAKLMNAYPSYISPIGCLPAHLLGDMWGRFWTN | |
| | LYSLTVPFGQKPNIDVTDAMVDQAWDAQRIFKEAEKFFVSVGLPNMTQGFWE | |
| | YSMLTDPGNVQKAVCHPTAWDLGKGDFRILMCTKVTMDDFLTAHHEMGHIQ | |
| | YDMAYAAQPFLLRNGANEGFHEAVGEIMSLSAATPKHLKSIGLLSPDFQEDNE | |
| | TEINFLLKQALTIVGTLPFTYMLEKWRWMVFKGEIPKDQWMKKWWEMKREIV | |
| | GVVEPVPHDETYCDPASLFHVSNDYSFIRYYTRTLYQFQFQEALCQAAKHEG | |
| | PLHKCDISNSTEAGQKLFNMLRLGKSEPWTLALENVVGAKNMNVRPLLNYFE | |
| | PLFTWLKDQNKNSFVGWSTDWSPYADQSIKVRISLKSALGDKAYEWNDNEM | |
| | YLFRSSVAYAMRQYFLKVKNQMILFGEEDVRVANLKPRISFNFFVTAPKNVSDI | |
| | IPRTEVEKAIRMSRSRINDAFRLNDNSLEFLGIQPTLGPPNQPPVSGGGGSGG | |
| | GGSDKTHTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVKGFYPS | |
| | DIAVEWESNGQPENNYKTTPPVLDSDGSFFLYSKLTVDKSRWQQGNVFSCS | |
| | VMHEALHNHYTQKSLSLSPGKGGGGSGGGGSGGGGSGGGGSQSTIEEQAK | |
| | YFLDKFNHEAEDLFYQSSLASWNYNTNITEENVQNMNNAGDKWSAFLKEQST | |
| | TAQMYPLQEIQNLTVKLQLQALQQNGSSVLSEDKSKRLNTILNTMSTIYSTGKV | |
| | CNPDNPQECLLLEPGLNEIMANSLDYNERLWAWESWRSEVGKQLRPLYEEY | |
| | VVLKNEMARANHYEDYGDYWRGDYEVNGVDGYDYSRGQLIEDVEHTFEEIK | |
| | PLYEHLHAYVRAKLMNAYPSYISPIGCLPAHLLGDMWGRFWTNLYSLTVPFG | |
| | QKPNIDVTDAMVDQAWDAQRIFKEAEKFFVSVGLPNMTQGFWEYSMLTDPG | |
| | NVQKAVCHPTAWDLGKGDFRILMCTKVTMDDFLTAHHEMGHIQYDMAYAAQ | |
| | PFLLRNGANEGFHEAVGEIMSLSAATPKHLKSIGLLSPDFQEDNETEINFLLKQ | |
| | ALTIVGTLPFTYMLEKWRWMVFKGEIPKDQWMKKWWEMKREIVGVVEPVPH | |
| | DETYCDPASLFHVSNDYSFIRYYTRTLYQFQFQEALCQAAKHEGPLHKCDISN | |
| | STEAGQKLFNMLRLGKSEPWTLALENVVGAKNMNVRPLLNYFEPLFTWLKDQ | |
| | NKNSFVGWSTDWSPYAD | |
| ACE2 (615/615)- | Chain 1: ACE2 (615) - Linker - lgG Fc - Linker - | N/A |
| lgG-Fc | ACE2 (615) | |
| | Chain 2: ACE2 (615) - Linker - lgG Fc - Linker - | |
| | ACE2 (615) | |
| ACE2 (615/615) | Chain 1: ACE2 (615) v2.4 - Linker - lgG Fc - Linker - | N/A |
| v2.4-lgG-Fc | ACE2 (615) v2.4 | |
| | Chain 2: ACE2 (615) v2.4 - Linker - lgG Fc - Linker - | |
| | ACE2 (615) v2.4 | |
| ACE2 | Chain 1: ACE2 (740) - Linker - CH1 - Linker - lgG | N/A |
| (740/615)Hex - | Fc - Linker - ACE2 (615) | |
| lgG-Fc | Chain 2: ACE2 (740) - Linker - CH1 - Linker - lgG | |
| (also “ACE2 | Fc - Linker - ACE2 (615) | |
| (740/615)-Fc | Chain 3: ACE2 (740) - Linker - CL | |
| (x6)” herein) | Chain 4: ACE2 (740) - Linker - CL | |
| ACE2 | Chain 1: ACE2 (740) v2.4 - Linker - CH1 - Linker - | N/A |
| (740/615)Hex | lgG Fc - Linker - ACE2 (615) v2.4 | |
| v2.4-lgG-Fc | Chain 2: ACE2 (740) v2.4 - Linker - CH1 - Linker - | |
| (also “ACE2 v2.4 | lgG Fc - Linker - ACE2 (615) v2.4 | |
| (740/615)-Fc | Chain 3: ACE2 (740) v2.4 - Linker - CL | |
| (x6)” herein) | Chain 4: ACE2 (740) v2.4 - Linker - CL | |
| ACE2 | Chain 1: ACE2 (740) - Linker - CH1 - Linker - lgG | N/A |
| (740/615)Octa- | Fc - Linker - ACE2 (615) | |
| lgG-Fc | Chain 2: ACE2 (740) - Linker - CH1 - Linker - lgG | |
| (also “ACE2 | Fc - Linker - ACE2 (615) | |
| (740/615)-Fc | Chain 3: ACE2 (740) - Linker - CL - Linker - | |
| (x8)” herein) | ACE2 (615) | |
| | Chain 4: ACE2 (740) - Linker - CL - Linker - | |
| | ACE2 (615) | |
| ACE2 | Chain 1: ACE2 (740) v2.4 - Linker - CH1 - Linker - | N/A |
| (740/615)Octa | lgG Fc - Linker - ACE2 (615) v2.4 | |
| v2.4-lgG-Fc | Chain 2: ACE2 (740) v2.4 - Linker - CH1 - Linker - | |
| (also “ACE2 v2.4 | lgG Fc - Linker - ACE2 (615) v2.4 | |
| (740/615)-Fc | Chain 3: ACE2 (740) v2.4 - Linker - CL - Linker - | |
| (x8)” herein) | ACE2 (615) v2.4 | |
| | Chain 4: ACE2 (740) v2.4 - Linker - CL - Linker - | |
| | ACE2 (615) v2.4 | |
| ACE2 (740)Tetra- | Chain 1: ACE2 (740) - Linker - CH1 - Linker - | N/A |
| lgG-Fc (4x) N4 | lgG Fc | |
| (also “ACE2 | Chain 2: ACE2 (740) - Linker - CH1 - Linker - | |
| (740)-Fc (4x) | lgG Fc | |
| N4” herein) | Chain 3: ACE2 (740) - Linker - CL | |
| | Chain 4: ACE2 (740) - Linker - CL | |
| ACE2 (740)Tetra- | Chain 1: ACE2 (740) v2.4 - Linker - CH1 - Linker - | N/A |
| v2.4-lgG-Fc N4 | lgG Fc | |
| (also “ACE2 | Chain 2: ACE2 (740) v2.4 - Linker - CH1 - Linker - | |
| (740) v2.4-Fc | lgG Fc | |
| (4x) N4” herein) | Chain 3: ACE2 (740) v2.4 - Linker - CL | |
| | Chain 4: ACE2 (740) v2.4 - Linker - CL | |
| ACE2 (740)Tetra- | Chain 1: lgG Fc - Linker - ACE2 (740) - Linker - | N/A |
| lgG-Fc C4 | CH1 | |
| (also “ACE2 | Chain 2: lgG Fc - Linker - ACE2 (740) - Linker - | |
| (740)-Fc (4x) | CH1 | |
| C4” herein) | Chain 3: ACE2 (740) - Linker - CL | |
| | Chain 4: ACE2 (740) - Linker - CL | |
| ACE2 (740)Tetra- | Chain 1: lgG Fc - Linker - ACE2 (740) v2.4 - | N/A |
| v2.4-lgG-Fc C4 | Linker - CH1 | |
| (also “ACE2 | Chain 2: lgG Fc - Linker - ACE2 (740) v2.4 - | |
| (740) v2.-Fc | Linker - CH1 | |
| (4x) C4” herein) | Chain 3: ACE2 (740) v2.4 - Linker - CL | |
| | Chain 4: ACE2 (740) v2.4 - Linker - CL | |
| ACE2 (740)Octa- | Chain 1: ACE2 (740) - Linker - CH1 - Linker - lgG | N/A |
| lgG-Fc N4/C4 | Fc - Linker - ACE2 (740) - Linker - CH1 | |
| (also “ACE2 | Chain 2: ACE2 (740) - Linker - CH1 - Linker - lgG | |
| (740)-Fc (8x) | Fc - Linker - ACE2 (740) - Linker - CH1 | |
| N4/C4” herein) | Chain 3: ACE2 (740) - Linker - CL | |
| | Chain 4: ACE2 (740) - Linker - CL | |
| | Chain 5: ACE2 (740) - Linker - CL | |
| | Chain 6: ACE2 (740) - Linker - CL | |
| ACE2 (740)Octa- | Chain 1: ACE2 (740) v2.4 - Linker - CH1 - Linker - | N/A |
| v2.4.lgG-Fc | lgG Fc - Linker - ACE2 (740) v2.4 - Linker - CH1 | |
| N4/C4 (also | Chain 2: ACE2 (740) v2.4 - Linker - CH1 - Linker - | |
| “ACE2 (740) | lgG Fc - Linker - ACE2 (740) v2.4 - Linker - CH1 | |
| v2.4-Fc (8x) | Chain 3: ACE2 (740) v2.4 - Linker - CL | |
| N4/C4” herein) | Chain 4: ACE2 (740) v2.4 - Linker - CL | |
| | Chain 5: ACE2 (740) v2.4 - Linker - CL | |
| | Chain 6: ACE2 (740) v2.4 - Linker - CL | |

The expression plasmids containing the constructs of interest were used to transiently transfect CHO, Expi293, or FreeStyle™ 293-F cells (ThermoFisher) following the manufacturer's protocol. After 6 days, culture supernatants were harvested, centrifuged at 3900 rpm, 4° C. for 15 minutes, and filtered through 0.2 μm size filter for further purification. Some constructs were further purified with SEC and a Protein A affinity step. Further details about the two tetravalent constructs and additional exemplary constructs are presented in Table 4.

TABLE 4

| Construct | MW (kDa) | ACE2 range | Fc | Exp. Host | Exp. Vol (mL) | Yield (mg) |
|---|---|---|---|---|---|---|
| ACE2 (740/615)-Fc | 368 | 18-740aa, 18-615aa | IgG1 Fc | Expi 293 | 250 | 5.2 |
| ACE2 v2.4 (740/615)-Fc | 368 | 18-740aa v2.4, 18-615aa v2.4 | IgG1 Fc | Expi 293 | 250 | 4.3 |
| ACE2 (615/615)-Fc | 332 | 18-615aa | IgG1 Fc | Expi 293 | * | * |
| ACE2 v2.4 (615/615)-Fc | 332 | 18-615aa v2.4 | IgG1 Fc | Expi 293 | * | * |
| ACE2 (740/615)He x -Fc | 570 | 18-740aa, 18-615aa | IgG1 Fc | Freestyle 293 | * | * |
| ACE2 v2.4 (740/615)He x-Fc | 570 | 18-740aa v2.4, 18-615aa v2.4 | IgG1 Fc | Freestyle 293 | * | * |
| ACE2 (740/615)Oct a-Fc | 710 | 18-740aa, 18-615aa | IgG1 Fc | Freestyle 293 | * | * |
| ACE2 v2.4 (740/615)Oct a-Fc | 710 | 18-740aa v2.4, 18-615aa v2.4 | IgG1 Fc | Freestyle 293 | * | * |
| ACE2 (615)-Fc | 190 | 18-615aa | IgG1 Fc | CHO | * | * |
| ACE2 v2.4 (615)v2.4-Fc | 190 | 18-615aa v2.4 | IgG1 Fc | Expi293 | * | * |
| ACE2 (740)-Fc | 218 | 18-740aa | IgG1 Fc | Expi293 | * | * |

*25-30 mg/mL yield after Protein A affinity step.

### 8.1.2. Purification and Quality Assessment

Isolation of fusion protein from supernatant was performed by use of a 1 mL Mabselect Sure affinity resin-based column (Cytiva). First, the columns were equilibrated with 5 column volumes (CV) of the column equilibration buffer (50 mM Tris-HCl, 150 mM NaCl, pH7.5) a flow rate of 2.0 mL/min. Next, sterile filtered supernatant containing fusion proteins was loaded over the pre-equilibrated column at a flow rate of 2.0 mL/min. Any non-specifically bound materials were washed out of the column using 50 mM Tris-HCl, 500 mM NaCl, pH7.5 at a flow rate of 2.0 mL/min for 5 CV. The affinity-bound fusion protein was eluted from the column using Pierce™ IgG Elution Buffer (pH 2.8, Thermo Fisher) at a flow rate of 0.5 mL/min for 5 CV. The elution fraction was collected in a reservoir pre-filled with 1M Tris-HCl, pH8.0 neutralization buffer in a 1/10^{th} ratio of the total elution fraction volume. All buffers and samples used in the process were sterile filtered using 0.2 μm PES filters.

The neutralized elution fraction was evaluated by UV-Vis to determine its protein concentration using a Labchip Dropsense instrument. The fraction was further analyzed by SE-UPLC to determine the presence of high or low molecular weight species relative to the species of interest. The size exclusion chromatography (SEC) column utilized was the Acquity BEH, 200 Angstrom, 1.7 μm, 4.6×150 mm column (Waters), with a flow rate of 0.3 mL/min, in 1xDPBS, 0.5M NaCl, pH 7.1. The elution fraction material was further polished to increase the purity of the species of interest by SEC. Hence, a Superdex 200 10/300GL column (Cytiva) was employed at a flow rate of 0.75 mL/min, in 1xDPBS, 5% Glycerol, pH7.4 running buffer, and a total injection volume of 1 mL.

The SEC fractionation resulted in the isolation of three separate fraction pools. Each fraction pool was analyzed by UV-Vis to determine the protein concentration. Each fraction pool was further analyzed by SE-UPLC to determine the relative purity of the species of interest. The proteins isolated from each fraction pool were analyzed under denaturing conditions using SDS-PAGE. Furthermore, the samples were also run for 1 hour at 200 V constant on 4-20% Tris-Glycine gels that were loaded with 2 μg of sample per well. This is for patent paragraph numbering.

### 8.1.3. SARS-CoV2 Pseudovirus Neutralization Assay

Vero cells were cultured in DMEM high glucose medium with sodium pyruvate and without glutamine, supplemented with 10% heat-inactivated FBS and Penicillin/Streptomycin/L-glutamine (Complete DMEM) at 37° C. in 5% CO2 and seeded at 20,000 cells/well in 96-well black/clear bottom cell culture plates. On the day of the assay, test articles (antibodies and proteins) were diluted to 2X assay concentration and serially diluted 3-fold, for a total of 11 concentrations (e.g., 40 nM to 677.4 pM). All dilutions were performed using infection media consisting of DMEM high glucose medium without sodium pyruvate/with glutamine that was supplemented with Sodium Pyruvate, 0.2% IgG-free BSA, and Gentamicin.

The pVSV-Luc-SARS-CoV2-S pseudoviruses used herein were non-replicating VSV-DG, that expressed a dual GFP/firefly luciferase reporter in place of its native glycoprotein, and pseudotyped with SARS-CoV2 Spike. The pseudoviruses were diluted 1:4 in infection media, then combined 1:1 with test article dilutions for a final pseudovirus dilution of 1:8 and final test article concentrations of 20 nM to 338.7 pM. Wells containing no test articles (virus control) or no pseudoviruses (medium control) were used as controls. The combined test articles and pseudoviruses were incubated at room temperature for 30 minutes. Next, the culture media were removed from the cells and the combined test articles and pseudoviruses were added 100 uL/well in duplicates to the wells, which were then incubated at 37ºC, 5% CO2 for 24 hours. At 24 hours post-infection, media were removed from the wells, and the cells were lysed using 100 uL/well Glo-Lysis buffer (Promega). Immediately before reading luminescence on the Spectramax i3X plate reader, 100 uL prepared Bright-Glo substrate (Promega) was added to the lysates. The results were exported to Microsoft Excel, where % neutralization was calculated with the following equation: % Neutralization=((1−(well value − medium control)/(virus control − medium control))×100% Neutralization is then plotted in GraphPad Prism and analyzed using nonlinear regression: log(inhibitor) vs. response-Variable slope (four parameter) to calculate IC50 values.

### 8.2. Example 1: Production of Multivalent Fusion Proteins

The extracellular portion of the ACE2 protein consists of two main domains: the peptidase domain referred to herein as ACE2-PD or ACE2 (615) which corresponds to the amino acids 18 to 615 from the N-terminus, and part of the collectrin like domain (CLD) called neck domain, referred to herein as ACE2-ND, which corresponds to the amino acids 616 to 740 from the N-terminus (FIGS. 1A and 1B). ACE2-PD+ND with amino acids spanning from 18-740 from the N-terminus is also referred to as ACE2 (740) thereafter (FIG. 1B). The peptide binding cavity on the outer surface of ACE2-PD is directly involved in RBD binding whereas ACE2-ND is critical for ACE2 dimerization. Different ACE2 ectodomain constructs were devised herein by including or excluding ACE2-ND, as illustrated in FIG. 2.

The multivalent fusion proteins were designed and prepared as described in section 8.1.1. The transfected cells successfully expressed the multivalent fusion proteins contained in the vector with which they were transfected. SDS-PAGE analysis of culture medium samples collected from transfected cells indicates that the expression levels of ACE2-Fc or ACE2 v2.4-Fc constructs were comparable (FIG. 3).

### 8.3. Example 2: Purification of Tetravalent Fusion Proteins

The culture medium samples collected from transfected cells were filtered, isolated with affinity resin columns, and further purified with SEC columns as described in Section 8.1.2. The SEC fractionation resulted in the isolation of three separate fraction pools, F1, F2, and F3. After the protein concentration of each fraction pool was determined, the relative purity of each fraction was assessed via SE-UPLC and SDS-PAGE.

The SEC profiles of ACE2 (740/615)-Fc (IgG) and ACE2 v2.4 (740/615)-Fc (IgG) fractions F1, F2, and F3 were associated with the detection of the purified protein constructs in all three fractions. (FIGS. 4A and 4B). When the three fractions of ACE2 (740/615)-Fc (IgG) and ACE2 v2.4 (740/615)-Fc (IgG) were analyzed with non-reducing SDS-PAGE, the purified proteins were detected at a much higher molecular weight than the monomers detected at around 180 kDa with a reducing SDS-PAGE setup (FIGS. 4C and 4D), supporting the notion that the ACE2 fusion proteins form proper dimers of 360 kDa. Moreover, these gel images also illustrate the isolation of the highest amount of was achieved with fraction F3. Moreover, further analysis of the fraction pool F3 revealed that this fraction was associated with the largest removal of the front shoulder peak seen originally (FIGS. 4E and 4F). Based on these results, this fraction pool was determined to contain the species of interest with the highest purity and was used for the subsequent neutralization assays.

### 8.4. Example 3: Neutralization Activity of Multivalent ACE2-Fc Constructs Against SARS-CoV2 Variants and Pseudovirus.

Cell cultures and virus neutralization assays were conducted as described in Section 8.1.3 using the tetravalent constructs ACE v2.4 (740/615)-Fc (IgG) and ACE2 (740/615)-Fc (IgG) and the bivalent constructs ACE2 (615)-Fc (IgG), ACE v2.4 (615)-Fc (IgG), and ACE2 (740)-Fc (IgG).

Individual ACE2-Fc (IgG) constructs differed in their ability to neutralize viral substrates (FIG. 5 and Table 5). Among ACE2-Fc (IgG) constructs, ACE2 (615)-Fc (IgG) displayed the weakest neutralization against the SARS-CoV2 pseudovirus, D614G, whereas the neutralization potency of the tetravalent construct ACE2 (740/615)-Fc (IgG) was comparable to the potency of tetravalent ACE2 v2.4 (740/615)-Fc (IgG), both of which were superior to the potency displayed by the bivalent ACE2 v2.4 (615)-Fc (IgG) (FIG. 5A). Similar results were obtained when individual ACE2-Fc (IgG) constructs were tested against the SARS-CoV2 variants Omicron BA. 1 and BA.2 (FIGS. 5B and 5C, respectively), where the neutralization potency of the tetravalent construct ACE2 (740/615)-Fc (IgG) was comparable to the potency of tetravalent ACE2 v2.4 (740/615)-Fc (IgG) and similar or superior to the potency displayed by the bivalent ACE2 v2.4 (615)-Fc (IgG).

TABLE 5
Summary of Neutralization Activity of ACE2-Fc
(IgG) Constructs Against SARS-CoV2 Variants

| | | | | Fold reduction from against | |
|---|---|---|---|---|---|
| | IC50 (M) | | | D614G | |
| IgG Construct | D614G | Omicron BA.1 | Omicron BA.2 | Omicron BA.1 | Omicron BA.2 |
| ACE2 (615)-Fc | 1.94E−08 | N.D. | N.C. | N.C. | N.C. |
| ACE2 (740)-Fc | 9.81E−10 | 1.43E−10 | 3.26E−10 | 0.15 | 0.33 |
| ACE2 (740/615)-Fc | 1.35E−11 | 2.08E−11 | 6.62E−11 | 1.54 | 4.90 |
| ACE2 v2.4 (615)- | 1.05E−10 | 2.32E−11 | 6.96E−11 | 0.22 | 0.66 |
| Fc | | | | | |
| ACE2 v2.4 | 3.25E−11 | 2.72E−11 | 5.74E−11 | 0.84 | 1.77 |
| (740/615)-Fc | | | | | |

N.D.: Not determined
N.C.: Not calculated due to weak neutralization

### 8.5. Example 4: Neutralization Activity of Multivalent ACE2-Fc Constructs Against Additional SARS-CoV2 Variants and Pseudovirus

In this assessment, the ability of ACE2-Fc (IgG) constructs to neutralize viral substrates were conducted as described in Section 8.1.3, and included the ACE2-Fc (IgG) constructs evaluated in Section 8.4. and an additional tetravalent construct, ACE2 (615) x4, which has the configuration displayed in FIG. 2E. Furthermore, additional SARS-CoV2 variants Omicron BA.2.12.1, BA.4/BA.5, and BA.4.6 were included in the assessment.

As observed in the experiments described in Example 3, the bivalent constructs ACE2 (615)-Fc (x2) and ACE2 (740)-Fc (x2) displayed the weakest overall neutralization against the SARS-CoV2 pseudovirus, D614G and the SARS-CoV2 Omicron variants BA. 1 and BA.2, whereas the bivalent construct ACE v2.4 (615)-Fc (x2) was associated with stronger potency against these viral substrates (FIGS. 6A-6C). All three tetravalent constructs displayed similar potencies which were superior to the potency displayed by the bivalent ACE2 v2.4 (615)-Fc (x2) (FIGS. 6A-6C).

Similar results were obtained with the additional SARS-CoV2 Omicron variants BA.2.12.1, BA.4/BA.5, and BA.4.6, where the tetravalent constructs were associated with higher neutralization potencies than the potencies displayed by the bivalent constructs (FIGS. 6D-6F). Among the tetravalent constructs, ACE2 (740/615)-Fc (x4) and ACE2 (615)-Fc (x4) displayed equal or superior neutralization potencies than the potency associated with ACE2 v2.4 (740/615)-Fc (x4) (FIGS. 6D-6F). The results of this example are summarized in Table 6. Taken together, these results suggested that higher valency is generally associated with higher neutralization potency. Furthermore, affinity maturated ACE2 v2.4 seemed to boost potency for the bivalent constructs but had minimal effect on the potency of tetravalent constructs.

TABLE 6
Summary of Neutralization Activity of ACE2-Fc (IgG) Constructs Against Six SARS-CoV2 Variants

| | IC50 [M] | | | | | | Fold reduction from against D614G | | | | | |
|---|---|---|---|---|---|---|---|---|---|---|---|---|
| | | | | | BA.4/ | | | | | | BA.4/ | |
| IgG Construct | D614G | BA.1 | BA.2 | BA.2.12.1 | BA.5 | BA.4.6 | D614G | BA.1 | BA.2 | BA.2.12.1 | BA.5 | BA.4.6 |
| ACE2 (740/ | 6.31E−11 | 5.54E−12 | 1.78E−11 | 1.71E−11 | 3.35E−11 | 2.45E−11 | 11.91 | 1.05 | 3.36 | 3.23 | 6.32 | 4.62 |
| 615)-Fc (x4) | | | | | | | | | | | | |
| ACE2 v2.4 | 6.99E−11 | 7.18E−12 | 2.35E−11 | 2.75E−11 | 4.42E−11 | 4.59E−11 | 13.19 | 1.35 | 4.43 | 5.19 | 8.34 | 8.66 |
| (740/615)-Fc | | | | | | | | | | | | |
| (x4) | | | | | | | | | | | | |
| ACE2 (615)- | 1.03E−10 | 6.61E−12 | 1.86E−11 | 1.23E−11 | 6.17E−11 | 3.35E−11 | 19.44 | 1.25 | 3.51 | 2.32 | 11.64 | 6.32 |
| Fc (x4) | | | | | | | | | | | | |
| ACE2 v2.4 | 9.71E−11 | 8.85E−12 | 2.36E−11 | 2.12E−11 | 4.51E−11 | 4.32E−11 | 18.32 | 1.67 | 4.45 | 4.00 | 8.51 | 8.15 |
| (615)-Fc (x4) | | | | | | | | | | | | |
| ACE2 v2.4 | 1.79E−10 | 2.18E−11 | 5.97E−11 | 5.43E−11 | 1.14E−10 | 1.04E−10 | 33.78 | 4.11 | 11.27 | 10.25 | 21.51 | 19.63 |
| (615)-Fc (x2) | | | | | | | | | | | | |
| ACE2 v2.4 | 1.07E−09 | 1.5E−10 | 2.05E−10 | 2.61E−10 | 2.9E−10 | 2.24E−10 | 201.92 | 28.31 | 38.69 | 49.25 | 54.73 | 42.27 |
| (740)-Fc (x2) | | | | | | | | | | | | |
| ACE2 v2.4 | 2.73E−09 | 1.27E−09 | 2.73E−09 | 2.48E−09 | 1.68E−09 | 1.82E−09 | 515.19 | 239.67 | 515.19 | 468.01 | 317.04 | 343.46 |
| (615)-Fc (x2) | | | | | | | | | | | | |

### 8.6. Example 5: Neutralization Activity of Additional Multivalent ACE2-Fc Constructs Against SARS-CoV2 Variants and Pseudovirus

To determine whether increasing the valency of the ACE2-Fc (IgG) constructs would improve their potency to neutralize viral substrates, hexavalent and octavalent ACE2-Fc (IgG) constructs ACE2 (740/615)-Fc (x6) and ACE2 (740/615) (x8) were assessed as described in Section 8.1.3, and their potencies were compared to those of ACE2 (740/615)-Fc (x4), ACE2 (615)-Fc (x2), and ACE2 (740)-Fc (x2).

Against all evaluated variants, ACE2 (615)-Fc (x2), and ACE2 (740)-Fc (x2) displayed the weakest neutralization potencies (FIGS. 7A-7F and Table 7), which was consistent with the results obtained in Examples 3 and 4. The hexavalent and octavalent ACE2-Fc (IgG) constructs performed similar to or better than the tetravalent construct, ACE2 (740/615)-Fc (x4) (FIGS. 7A-7F and Table 7). Hexavalent and octavalent affinity-maturated ACE2 v2.4 comprising constructs ACE2 v2.4 (740/615)-Fc (x6) and ACE2 v2.4 (740/615)-Fc (x8) performed similarly to their wildtype ACE2 comprising counterparts ACE2 (740/615)-Fc (x6) and ACE2 (740/615)-Fc (x8) (Table 7). Taken together, these observations indicated that increasing the valency of ACE2-Fc (IgG) constructs generally boosts viral particle neutralization potencies. Affinity maturated ACE2 had limited impact on the viral particle neutralization potencies of the high valency constructs tested.

TABLE 7
Summary of Neutralization Activity of Hexavalent and Octavalent ACE2-Fc (IgG) Constructs Against SARS-CoV2 Variants

| | IC50 [M] | | | | | | Fold reduction from against D614G | | | | | |
|---|---|---|---|---|---|---|---|---|---|---|---|---|
| | | | | | BA.4/ | | | | | | BA.4/ | |
| IgG Construct | D614G | BA.1 | BA.2 | BA.2.12.1 | BA.5 | BA.4.6 | D614G | BA.1 | BA.2 | BA.2.12.1 | BA.5 | BA.4.6 |
| ACE2 (740/ | 1.66E−11 | 2.41E−12 | 9.99E−12 | 1.33E−11 | 2.31E−11 | 1.85E−11 | 3.13 | 0.45 | 1.89 | 2.51 | 4.36 | 3.49 |
| 615)-Fc (x6) | | | | | | | | | | | | |
| ACE2 v2.4 (740/ | 4.88E−11 | 3.42E−12 | 1.17E−11 | 1.36E−11 | 1.83E−11 | 2.24E−11 | 9.21 | 0.65 | 2.21 | 2.57 | 3.45 | 4.23 |
| 615)-Fc (x6) | | | | | | | | | | | | |
| ACE2 (740/ | 3.29E−11 | 4.78E−12 | 8.35E−12 | 9E−12 | 1.96E−11 | 2.13E−11 | 6.21 | 0.90 | 1.58 | 1.70 | 3.70 | 4.02 |
| 615)-Fc (x8) | | | | | | | | | | | | |
| ACE2 v2.4 (740/ | 9.93E−11 | 1.21E−11 | 3.26E−11 | 4.26E−11 | 6.67E−11 | 5.9E−11 | 18.74 | 2.28 | 6.15 | 8.04 | 12.59 | 11.13 |
| 615)-Fc (x8) | | | | | | | | | | | | |

### 8.7. Example 6: Effect of ACE2 Valency on Neutralization Activity of Multivalent ACE2-Fc Constructs Against Multiple SARS-CoV2 Variants and Pseudovirus

To confirm that the valency of the ACE2-Fc (IgG) constructs improves their potency to neutralize viral substrates and to evaluate their effectiveness against additional variants, tetravalent, hexavalent and octavalent ACE2-Fc (IgG) constructs ACE2 (740/615)-Fc (x4), ACE2 (740/615)-Fc (x6) and ACE2 (740/615) (x8) and their affinity-matured counterparts ACE2 v2.4 (740/615)-Fc (x4), ACE2 v2.4 (740/615)-Fc (x6) and ACE2 v2.4 (740/615) (x8) were assessed as described in Section 8.1.3, and their potencies were compared to those of ACE2 (615)-Fc (x2), ACE2 v2.4 (615)-Fc (x2), and ACE2 (740)-Fc (x2).

Consistent with the results obtained in Examples 3, 4, and 5, ACE2 (615)-Fc (x2) and ACE2 (740)-Fc (x2) displayed the weakest neutralization potencies against all variants (FIGS. 8A-8F and Table 8). As was observed in Example 5, hexavalent and octavalent ACE2-Fc (IgG) constructs ACE2 (740/615)-Fc (x6) and ACE2 (740/615) (x8) performed similar to or better than the tetravalent construct, ACE2 (740/615)-Fc (x4) (FIGS. 8A-8F and Table 8). Hexavalent and octavalent affinity-matured ACE2 v2.4 comprising constructs ACE2 v2.4 (740/615)-Fc (x6) and ACE2 v2.4 (740/615)-Fc (x8) performed similarly to their wildtype ACE2 comprising counterparts ACE2 (740/615)-Fc (x6) and ACE2 (740/615)-Fc (x8) (Table 8). Taken together, these observations provided further evidence that increasing the valency of ACE2-Fc (IgG) constructs generally boosts viral particle neutralization potencies.

TABLE 8
Summary of Neutralization Activity of ACE2-Fc (IgG)
Constructs Against SARS-CoV2 Pseudovirus and Variants

| | Neutralization IC50 [M] | | | | | |
|---|---|---|---|---|---|---|
| IgG Construct | D614G | BA.1 | BA.2 | BA.2.12.1 | BA.4/BA.5 | Alpha-468 |
| ACE2 (740/615)-Fc (x4) | 1.35E−11 | 2.08E−11 | 6.62E−11 | 2.50E−11 | 7.66E−11 | 1.54E−10 |
| ACE2 v2.4 (740/615)-Fc (x4) | 3.25E−11 | 2.72E−11 | 5.74E−11 | 4.47E−11 | 6.28E−11 | 1.32E−10 |
| ACE2 (740/615)-Fc (x6) | 4.81E−11 | 9.22E−12 | 1.48E−11 | 8.23E−12 | 3.89E−11 | 5.39E−11 |
| ACE2 v2.4 (740/615)-Fc (x6) | 4.72E−11 | 7.19E−12 | 1.89E−11 | 7.08E−12 | 3.32E−11 | 4.74E−11 |
| ACE2 (740/615)-Fc (x8) | 1.17E−12 | ND | 2.01E−11 | 5.33E−12 | 1.13E−11 | 5.25E−11 |
| ACE2 v2.4 (740/615)-Fc (x8) | 1.96E−11 | ND | 1.77E−11 | 1.88E−10 | 2.71E−10 | 2.76E−10 |
| ACE2 (615)-Fc (x2) | 1.94E−08 | ND | ND | ND | 6.63E−09 | ND |
| ACE2 v2.4 (615)-Fc (x2) | 1.05E−10 | 2.32E−11 | 6.96E−11 | 1.34E−10 | 2.71E−10 | 2.92E−10 |
| ACE2 (740)-Fc (x2) | 9.81E−10 | 1.43E−10 | 3.26E−10 | 8.98E−10 | 6.93E−10 | 5.06E−10 |

8.8. Example 7: Neutralization Potency of Additional Multivalent ACE2-Fc Constructs with High ACE2 Valency Against SARS-CoV2 Pseudovirus and XBB1.5 Variant

To determine whether the structural arrangement of multivalent ACE2-Fc (IgG) constructs affects their neutralization activity, tetravalent and octavalent ACE2-Fc (IgG) constructs with structural differences (e.g., spatial arrangement of ACE2 moieties in tetravalent constructs and length of linkers connecting ACE2 and multimerization moieties in octavalent constructs) were generated as described in Section 8.1.1 and assessed as described in Section 8.1.3. The potencies of the multivalent ACE2-Fc constructs against the SARS-CoV2 pseudovirus and XBB.1.5 variant were compared to those of ACE2 (740)-Fc (x2) and REGN10933+REGN10987 (REGEN-COV).

Consistent with the results of the Examples 5 and 6, higher valency ACE2-Fc (IgG) constructs displayed a higher potency against the SARS-CoV2 pseudovirus D614G and the XBB1.5 variant, in which the bivalent construct ACE2 (740)-Fc (x2) displayed the weakest neutralization potency among the tested ACE2-Fc constructs (FIGS. 9B-9D and Table 9). More specifically, the hexavalent ACE2-Fc (IgG) construct was associated with a neutralization potency against the pseudovirus similar to that of REGEN-COV, which is an antibody cocktail effective against pre-Omicron SARS-CoV2 variants. The neutralization potency of the hexavalent ACE2-Fc (IgG) construct was higher than the neutralization potencies of tetravalent constructs and comparable to the potencies of octavalent constructs (FIGS. 9B-9D and Table 9). Among tetravalent constructs, the construct with four N-terminal ACE2 moieties, ACE2 (740)-Fc (4x) N4, displayed the best neutralization potency against the pseudovirus at a level approaching the neutralization potency displayed by hexavalent and octavalent ACE2-Fc (IgG) fusion proteins (FIGS. 9B-9D, and Table 9). Similarly, the neutralization potency of the hexavalent ACE2-Fc (IgG) construct against the XBB1.5 variant was higher than the neutralization potencies of tetravalent constructs and comparable to the potencies of octavalent constructs (FIGS. 9E-9G and Table 9). Among tetravalent constructs, the construct with four N-terminal ACE2 moieties, ACE2 (740)-Fc (4x) N4, displayed the best neutralization potency against the XBB1.5 variant, at a level approaching the neutralization potency displayed by hexavalent and octavalent ACE2-Fc (IgG) fusion proteins (FIGS. 9E-9G and Table 9). REGEN-COV displayed no neutralization potency against the XBB1.5 variant.

Taken together, these results suggested that ACE2-Fc (IgG) fusion proteins with a valency equal to or greater than 4 were effective against both the SARS-CoV2 pseudovirus and the XBB1.5 variant, with an additive effect by hexavalent and octavalent constructs over tetravalent constructs.

TABLE 9
Summary of Neutralization Activity of ACE2-Fc (IgG) Constructs
Against SARS-CoV2 Pseudovirus and XBB1.5 Variant

| | | | | | ACE2 | Fold Change |
|---|---|---|---|---|---|---|
| | | | | | Valency | over Parental |
| | | ACE2 | | Fold Change | Adjusted | (ACE2 Valency |
| | Construct | Valency | IC50 (M) | over Parental | IC50 (M) | Adjusted) |
| D614G | REGEN-COV | | 5.203E−12 | | | |
| | ACE2 (740)-Fc (2x) N2 | 2 | 6.164E−09 | 1.0 | 1.233E−08 | 1.0 |
| | ACE2 (740)-Fc (4x) N4 | 4 | 1.224E−11 | 503.6 | 4.897E−11 | 251.8 |
| | ACE2 (740)-Fc (4x) C4 | 4 | 8.008E−11 | 77.0 | 3.203E−10 | 38.5 |
| | ACE2 (740)-Fc (4x) N2/C2 | 4 | 5.346E−11 | 115.3 | 2.138E−10 | 57.7 |
| | ACE2 (740/615)-Fc (6x) N4/C2 | 6 | 4.512E−12 | 1366.1 | 2.707E−11 | 455.5 |
| | ACE2 (740)-Fc (8x) N4/C4 (long linker) | 8 | 4.644E−12 | 1327.3 | 3.715E−11 | 331.9 |
| | ACE2 (740)-Fc (8x) N4/C4 (medium linker) | 8 | 4.31E−13 | 14301.6 | 3.448E−12 | 3576.0 |
| | ACE2 (740)-Fc (8x) N4/C4 (short linker) | 8 | 2.247E−12 | 2743.2 | 1.797E−11 | 686.1 |
| XBB1.5 | REGEN-COV | | >2E−08 | | | |
| | ACE2 (740)-Fc (2x) N2 | 2 | 5.544E−09 | 1.0 | 1.109E−08 | 1.0 |
| | ACE2 (740)-Fc (4x) N4 | 4 | 7.191E−11 | 77.1 | 2.876E−10 | 38.6 |
| | ACE2 (740)-Fc (4x) C4 | 4 | 1.778E−10 | 31.2 | 7.111E−10 | 15.6 |
| | ACE2 (740)-Fc (4x) N2/C2 | 4 | 9.869E−11 | 56.2 | 3.947E−10 | 28.1 |
| | ACE2 (740/615)-Fc (6x) N4/C2 | 6 | 1.307E−11 | 424.2 | 7.84E−11 | 141.5 |
| | ACE2 (740)-Fc (8x) N4/C4 (long linker) | 8 | 3.333E−11 | 166.3 | 2.667E−10 | 41.6 |
| | ACE2 (740)-Fc (8x) N4/C4 (medium linker) | 8 | 4.471E−12 | 1240.0 | 3.577E−11 | 310.0 |
| | ACE2 (740)-Fc (8x) N4/C4 (short linker) | 8 | 1.611E−11 | 344.1 | 1.289E−10 | 86.0 |

### 8.9. Example 8: Effect of ACE2 Valency on Neutralization Potency of Multivalent ACE2-Fc Constructs Against SARS-CoV2 Variants BA.2.86 and EG.5.1

To further assess the effect of ACE2 valency of the ACE2-Fc (IgG) constructs on their potency to neutralize additional SARS-CoV2 variants BA.2.86 and EG.5.1, bivalent, tetravalent, and hexavalent ACE2-Fc (IgG) constructs ACE2 (740)-Fc (2x) N2, ACE2 (740)-Fc (4x) N2/C2, ACE2 (740)-Fc (4x) N4, and ACE2 (740/615) (6x) N4/C2 were assessed as described in Section 8.1.3, and their viral particle neutralization potencies were compared to the potency of REGN10933+REGN10987 (REGEN-COV), which is an antibody cocktail effective against pre-Omicron SARS-CoV2 variants.

REGEN-COV displayed no neutralization potency against either SARS-CoV2 variant. Against both variants, ACE2 (740)-Fc (2x) N2 displayed the weakest neutralization potency (FIGS. 10B and 10C). There was an ACE2 valency associated increase in viral particle neutralization potency, whereas hexavalent ACE2-Fc (IgG) construct ACE2 (740/615) (6x) N4/C2 performed better than the other constructs (FIGS. 10B and 10C). However, tetravalent constructs displayed the most dramatic enhancement, whereas the viral particle neutralization potency increase from tetravalent to hexavalent constructs was moderate. Furthermore, the viral particle neutralization potency of the tetravalent construct ACE2 (740)-Fc (4x) N4 was remarkably higher than that of the tetravalent construct ACE2 (740)-Fc (4x) N2/C2 (FIGS. 10B and 10C). Taken together, these results highlighted that both ACE2 valency and structural arrangement of ACE2 moieties affect the neutralization potency of ACE-2-Fc (IgG) constructs.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An ACE2 fusion protein comprising one or more polypeptide chains having the formula: 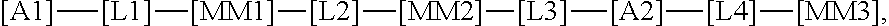
(a) [A1] represents an ACE2 moiety;
(b) [L1] represents a linker;
(c) [MM1] represents a multimerization moiety;
(d) [L2] represents a linker;
(e) [MM2] represents a multimerization moiety;
(f) [L3] represents a linker;
(g) [A2] represents an ACE2 moiety;
(h) [L4] represents a linker; and
(i) [MM3] represents a multimerization moiety,
and wherein:
(i) each of [L1], [L2], [L3] and [L4] may be absent;
(ii) one or both of [MM1] and [MM3] may be absent;
(iii) one but not both of [A1] and [A2] may be absent; and
(iv) [MM2] is present and is an IgG1 PVA Fc domain.

2. The ACE2 fusion protein of claim 1, wherein [A1] is present.

3. The ACE2 fusion protein of claim 2, wherein [A1] comprises an amino acid sequence having at least 90% sequence identity to the PD of ACE2 (SEQ ID NO:2).

4. The ACE2 fusion protein of claim 3, wherein [A1] comprises a ND.

5. The ACE2 fusion protein of claim 4, wherein [A1] comprises an amino acid sequence having at least 90% sequence identity to the PD+ND of ACE2 (SEQ ID NO:3).

6. The ACE2 fusion protein of claim 2, wherein [A1] comprises at least one amino acid substitution at position 25, 27, 31, 34, 42, 79, 90, 92, 324, 325, 330, or 386 of ACE2.

7. The ACE2 fusion protein of claim 2, wherein [A1] comprises the amino acid substitutions T27Y, L79T, and N330Y.

8. The ACE2 fusion protein of claim 2, wherein [A1] has an increase in affinity to a coronavirus RBD, wherein the increase in affinity is at least 25%, at least 50%, at least 100%, at least 200% or at least 300% as compared to the corresponding sequence in wildtype ACE2 (SEQ ID NO:1).

9. The ACE2 fusion protein of claim 1, in which [L1] is present and optionally is 5-35 amino acids in length.

10. The ACE2 fusion protein of claim 1, in which [MM1] is absent.

11. The ACE2 fusion protein of claim 1, in which [MM1] is present.

12. The ACE2 fusion protein of claim 11, wherein [MM1] is a CL domain.

13. The ACE2 fusion protein of claim 11, wherein [MM1] is a CH1 domain.

14.-19. (canceled)

20. The ACE2 fusion protein of claim 1, in which [L2] is present and is 5-35 amino acids in length.

21.-28. (canceled)

29. The ACE2 fusion protein of claim 1, in which [L3] is present and is 5-35 amino acids in length.

30. The ACE2 fusion protein of claim 1, wherein [A2] is present.

31. The ACE2 fusion protein of claim 30, wherein [A2] comprises an amino acid sequence having at least 90% sequence identity to the PD of ACE2 (SEQ ID NO:2).

32. The ACE2 fusion protein of claim 30, wherein [A2] comprises a ND.

33. The ACE2 fusion protein of claim 32, wherein [A2] comprises an amino acid sequence having at least 90% sequence identity to the PD+ND of ACE2 (SEQ ID NO:3).

34. The ACE2 fusion protein of claim 30, wherein [A2] comprises at least one amino acid substitution at position 25, 27, 31, 34, 42, 79, 90, 92, 324, 325, 330, or 386 of ACE2.

35. The ACE2 fusion protein of claim 30, wherein [A2] comprises the amino acid substitutions T27Y, L79T, and N330Y.

36. The ACE2 fusion protein of claim 30, wherein [A2] has an increase in affinity to a coronavirus RBD, wherein the increase in affinity is at least 25%, at least 50%, at least 100%, at least 200% or at least 300% as compared to the corresponding sequence in wildtype ACE2 (SEQ ID NO:1).

37. The ACE2 fusion protein of claim 1, in which [L4] is present.

38. The ACE2 fusion protein of claim 1, in which [MM3] is absent.

39. The ACE2 fusion protein of claim 1, in which [MM3] is present.

40. The ACE2 fusion protein of claim 39, wherein [MM3] is a CL domain.

41. The ACE2 fusion protein of claim 39, wherein [MM3] is a CH1 domain.

42. The ACE2 fusion protein of claim 1, which comprises:
a first polypeptide chain having the formula: [A1]-[L1]-[MM1] (“Formula 4”), wherein [MM1] is present and represents a CH1 domain or CL domain, and
a second polypeptide having the formula [A1]-[L1]-[MM1]-[L2]-[MM2] (“Formula 5”), wherein [MM1] is present and represents a CH1 domain or CL domain capable of pairing with [MM1] on the first polypeptide.

43. The ACE2 fusion protein of claim 42, wherein the second polypeptide chain has the formula [A1]-[L1]-[MM1]-[L2]-[MM2]-[L3]-[A2] (“Formula 6”), wherein [A2] is present and represents an ACE2 moiety.

44. The ACE2 fusion protein of claim 42, wherein [A1] and/or [A2] comprises the amino acid substitutions T27Y, L79T, and N330Y.

45. An ACE2 fusion protein comprising one or more polypeptide chains having the formula: 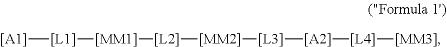
wherein:
(a) [A1] represents an ACE2 moiety;
(b) [L1] represents a linker;
(c) [MM1] represents a multimerization moiety;
(d) [L2] represents a linker;
(e) [MM2] represents a multimerization moiety;
(f) [L3] represents a linker;
(g) [A2] represents an ACE2 moiety;
(h) [L4] represents a linker; and
(i) [MM3] represents a multimerization moiety,
and wherein:
(i) each of [L1], [L2], [L3] and [L4] may be absent;
(ii) one or two but not all three of [MM1], [MM2] and [MM3] may be absent;
(iii) one but not both of [A1] and [A2] may be absent; and
(iv) at least one of [A1] and [A2] is present and comprises the amino acid substitutions T27Y, L79T, and N330Y.

46. A nucleic acid or plurality of nucleic acids encoding the ACE2 fusion protein of claim 1 or 45.

47. A host cell engineered to express the ACE2 fusion protein of claim 1 or 45.

48. A method of producing an ACE2 fusion protein of, comprising culturing the host cell of claim 47 and recovering the ACE2 fusion protein expressed thereby.

49. A pharmaceutical composition comprising the ACE2 fusion protein of claim 1 or 45 and an excipient.

50. A method of treating a coronavirus disease, inhibiting an interaction between a RBD of a coronavirus and cellular ACE2, reducing the severity of coronavirus infection, reducing the viral load of a coronavirus, preventing disease progression in a subject with a coronavirus infection, or reducing the risk of severe disease or death in a subject with a coronavirus infection, the method comprising administering to a subject in need thereof the ACE2 fusion protein of claim 1 or 45, wherein the coronavirus is SARS-CoV-2.
